SEQUENCE LISTING (2 sequences)

SEQ ID NO: 1 - DNA, 20 bp, Artificial Sequence
  Chemically synthesized forward primer
gcaatgcatcatgtgtgcta

SEQ ID NO: 2 - DNA, 21 bp, Artificial Sequence
  Chemically synthesized reverse primer
gtaaatgtcgaattcccttcg

(19) United States
(12) Patent Application Publication
(10) Pub. No.: US 2012/0317674 A1
(43) Pub. Date: Dec. 13, 2012

(54) HYBRID TOMATOES AND METHODS OF MAKING HYBRID TOMATOES

(75) Inventors: Harry J. Klee, Gainsville, FL (US); Denise Tieman, Gainesville, FL (US)

(73) Assignee: UNIVERSITY OF FLORIDA RESEARCH FOUNDATION, INC., Gainesville, FL (US)

(21) Appl. No.: 13/491,688

(22) Filed: Jun. 8, 2012

Related U.S. Application Data
(60) Provisional application No. 61/495,555, filed on Jun. 10, 2011.
(60) Provisional application No. 61/650,555, filed on May 23, 2012.

(51) Int. Cl. A01H 5/08; A01H 1/02; G01N 33/02; A01H 5/00

(52) U.S. Cl. 800/260; 800/317.4; 73/865.7

(57) ABSTRACT

The present disclosure provides hybrid tomatoes that produce better-tasting fruit, methods of producing and identifying the hybrid tomatoes and methods of identifying the chemical composition of a tomato that leads to a better-tasting fruit.

### CROSS-REFERENCE TO RELATED APPLICATIONS

This application claims priority to U.S. provisional applications entitled, “Hybrid Tomatoes and Methods of Making Hybrid Tomatoes,” having Ser. No. 61/495,555, filed on Jun. 10, 2011, and “Hybrid Tomatoes and Methods of Making Hybrid Tomatoes,” having Ser. No. 61/650,555, filed on May 23, 2012, both of which are entirely incorporated herein by reference.

### STATEMENT REGARDING FEDERALLY SPONSORED RESEARCH OR DEVELOPMENT

This invention(s) was made with government support under Grant No.: IOS-0923312 awarded by the National Science Foundation. The government has certain rights in the invention(s).

### BACKGROUND

The tomato is one of the most widely grown and valuable fruit crops world-wide. Despite its popularity and important contribution to human nutrition, consumers widely view the commercially produced fruit as having poor taste. Tomato flavor is a major source of consumer dissatisfaction. Intensive breeding for increased yield has led to erosion of flavor and nutrient content. Improvement or even maintenance of flavor has not been possible, in large part due to the complex nature of the trait.

Heirloom tomato varieties are grown by small and/or local producers and are generally perceived to have better taste than many of the commercially produced tomatoes. However, though such heirloom tomatoes are popular among home-growers and at local markets, many of these heirloom varieties are not sufficiently hardy in the field or in commerce for large-scale commercial production.

### SUMMARY

Briefly described, embodiments of the present disclosure provide for hybrid tomatoes that produce better-tasting fruit, methods of making the hybrid tomatoes and methods of identifying the hybrid tomatoes.

The present disclosure describes methods of identifying hybrid tomato plants that produce better-tasting fruit including the steps of: providing tomato samples from a plurality of different tomato plant varieties to a tasting panel and accumulating results of the tasting panel, where each panel member assigns a liking score to each tomato tested. The methods also include performing a chemical analysis of a tomato from each of the variety of tomatoes tested by the panel by quantifying an amount of a plurality of flavor-associated compounds from each tomato, where the flavor-associated compounds are chosen from sugars, acids, and volatile compounds and where at least one of the flavor-associated compounds quantified is a volatile compound. The methods for identifying hybrid tomato plants that produce better-tasting fruit further include correlating the results of the tasting panel scores with the calculated amounts of flavor-associated compounds for each tomato from the chemical analysis to determine which volatile compounds are positively associated with liking and which volatile compounds are negatively associated with liking, determining criteria for a better-tasting tomato based on the correlations between liking scores and the chemical content of a tomato, and identifying a hybrid tomato plant that produces fruit having at least one of the criterion for a better-tasting tomato.

Embodiments of hybrid tomato plants of the present disclosure include hybrid tomato plants that produce tomato fruit having a greater amount of at least one volatile compound positively associated with liking than the amount of that volatile compound in fruit produced by an ancestor elite tomato cultivar, where the volatile compound positively associated with liking is chosen from: 1-penten-3-one, trans-2-pentenal, trans-2-heptenal, trans-3-hexen-1-ol, trans-2-hexenal, cis-2-penten-1-ol, 6-methyl-5-hepten-2-ol, nonyl aldehyde, isovaleronitrile, cis-4-decenal, 3-methyl-1-butanol, 2,5-dimethyl-4-hydroxy-3(2H)-furanone, 1-pentanol, methional, benzyl cyanide, isovaleraldehyde, 3-pentanone, 2-isobutylthaizole, benzaldehyde, isovaleric acid, 1-nitro-3-methylbutane, β-ionone, β-cyclocitral, 6-methyl-5-hepten-2-one, geranial, phenylacetaldehyde, geranylacetone, 2-phenyl ethanol, or 1-octen-3-one, or any combination of two or more of these volatile compounds.

Additional embodiments of hybrid tomato plants of the present disclosure include F1 hybrid tomato plants produced by crossing an heirloom tomato cultivar and a parent of an elite hybrid cultivar, where the F1 hybrid tomato plant produces fruit that has a higher amount of at least one volatile compound positively associated with liking than the amount of that volatile compound present in fruit produced by the elite hybrid cultivar.

In further embodiments, the present disclosure provides hybrid tomato plants that produce tomato fruit including the following volatile compounds in about the following amounts, measured as volatile emission (ng gFW⁻¹h⁻¹):

about 1.6 ng gFW⁻¹h⁻¹ or more of 1-penten-3-one,

about 1.1 ng gFW⁻¹h⁻¹ or more of trans-2-pentenal,

about 1.2 ng gFW⁻¹h⁻¹ or more of trans-3-hexen-1-ol,

about 14.0 ng gFW⁻¹h⁻¹ or more of isovaleronitrile,

about 39.1 ng gFW⁻¹h⁻¹ or more of 3-methyl-1-butanol,

about 19.1 ng gFW⁻¹h⁻¹ or more of 1-nitro-3-methylbutane,

about 4.2 ng gFW⁻¹h⁻¹ or more of 6-methyl-5-hepten-2-one,

about 0.15 ng gFW⁻¹h⁻¹ or more of geranial,

about 0.06 ng gFW⁻¹h⁻¹ or more of 1-octen-3-one,

about 4.6 ng gFW⁻¹h⁻¹ or more of trans-2-hexenal,

about 1.2 ng gFW⁻¹h⁻¹ or more of cis-2-penten-1-ol,

about 0.48 ng gFW⁻¹h⁻¹ or less of eugenol,

about 3.9 ng gFW⁻¹h⁻¹ or less of 2-methylbutanal,

about 0.17 ng gFW⁻¹h⁻¹ or less of butylacetate, and

about 0.95 ng gFW⁻¹h⁻¹ or less of isobutylacetate.

In embodiments, the present disclosure also includes hybrid tomato plants produced by backcrossing a hybrid descendent of an ancestor heirloom tomato cultivar and an ancestor elite cultivar with one of the ancestor cultivars, where the hybrid tomato plant produces fruit that has a higher amount of at least one volatile compound positively associated with liking than the amount of that volatile compound present in fruit produced by the ancestor elite cultivar.

The present disclosure also provides embodiments of methods of making hybrid tomato plants including: crossing a parent of an elite hybrid tomato cultivar with an heirloom tomato cultivar, where the heirloom tomato cultivar produces tomato fruit with a greater amount of at least one volatile compound positively associated with liking than the elite hybrid tomato cultivar, to produce an F1 hybrid tomato plant that produces tomato fruit with a greater amount of the at least one volatile compound positively associated with liking than the elite hybrid tomato cultivar.

Embodiments of methods of the present disclosure also include methods of identifying a tomato plant that produces better tasting tomato fruit. In embodiments, such methods include the steps of: performing a chemical analysis of a tomato fruit from each of a variety of tomato plants, where the chemical analysis comprises quantifying an amount of at least one volatile compounds chosen from the compounds: 1-penten-3-one, trans-2-hexenal, cis-2-penten-1-ol, geranial, 3-methyl-1-butanol, 1-octen-3-one, trans-2-pentenal, isovaleronitrile, trans-3-hexen-1-ol, 1-nitro-3-methylbutane, 6-methyl-5-hepten-2-one, 2-methylbutanal, butyl acetate, isobutylacetate, and eugenol; and selecting the tomato plant that produced fruit having the greatest amount of one or more of the compounds positively associated with liking chosen from the compounds: 1-penten-3-one, trans-2-hexenal, cis-2-penten-1-ol, geranial, 3-methyl-1-butanol, 1-octen-3-one, trans-2-pentenal, isovaleronitrile, trans-3-hexen-1-ol, 1-nitro-3-methylbutane, and 6-methyl-5-hepten-2-one.

Other compositions, plants, methods, features, and advantages of the present disclosure will be or become apparent to one with skill in the art upon examination of the following drawings and detailed description. It is intended that all such additional compositions, plants, methods, features, and advantages be included within this description, and be within the scope of the present disclosure

### BRIEF DESCRIPTION OF THE DRAWINGS

Further aspects of the present disclosure will be more readily appreciated upon review of the detailed description of its various embodiments, described below, when taken in conjunction with the accompanying drawings.

FIGS. 1A-1C are graphs illustrating the following: C6 volatile emission in fruit of control (M82) and LoxC antisense plants (FIG. 1A); lack of significant correlation of overall liking with emission of cis-3-hexenal of examined heirloom varieties (FIG. 1B); high correlation of overall liking to levels of trans-2-pentenal in tomato fruit of examined heirloom varieties (FIG. 1C). Open and grey squares indicate the levels of volatile emission for the most liked and the idealized tomato, respectively, determined by regression analysis of overall liking of heirloom tomato varieties vs. volatiles emission levels (Table 2).

FIGS. 2A-2GG are a series of graphs illustrating the contribution of flavor-associated compounds to overall liking of tomatoes. The graphs depict linear regression analysis of overall liking rating vs. concentration of biochemical components of tomato flavor. The shaded square on each graph indicates concentrations found in the ideal recipe at highest panel rating (liking score of 34). The open square on each graph are concentrations found in the ideal recipe of the best tomato ever tasted by the panelists (liking score of 40) (shown in Table 2).

FIG. 3 illustrates a cluster analysis of tomato varieties sorted by flavor chemical composition. Varieties were sorted using JMP software on the basis of the measured basis of the 70 measured chemical attributes shown across the bottom. The names of varieties (right) and their consumer liking scores (left) are shown. Several varieties were tested in multiple seasons.

FIGS. 4A-C are graphs illustrating the genetic distribution of 19 heirloom cultivars that vary in liking (4A), sweetness (4B) and tomato flavor intensity (4C) scores. Genetic variation was determined using 27 polymorphic DNA markers, and the cultivars were clustered using principal components analysis. Each circle represents a cultivar with the number corresponding to its name. The color gradient corresponds with the liking score and varies from dark green (highly liked) to red (highly disliked). The dark circle in the bottom left of each plot corresponds to cultivars Chadwick Cherry and Large Red Cherry that were genetically indistinguishable but differed in consumer preferences.

FIGS. 5A and 5B illustrate ordered correlation matrices of flavor-associated fruit chemicals. FIG. 5A shows correlations of the 71 measured chemicals, and FIG. 5B shows correlations of the 27 selected for multivariate analysis. MMC (Stone et al., 2009) was used as a visual aid to assist in grouping closely related chemicals.

FIG. 6 is a graph illustrating the association of some volatile compounds to aroma liking; the first 10 volatile compounds listed are the top 10 positively associated with aroma liking.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

Any publications and patents cited in this specification that are incorporated by reference are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of agriculture, botany, statistics, organic chemistry, biochemistry, molecular biology, and the like, which are within the skill of the art. Such techniques are explained fully in the literature.

It must be noted that, as used in the specification and the appended embodiments, the singular forms “a,” “an,” and “the” include plural referents unless the context clearly dictates otherwise. Thus, for example, reference to “a support” includes a plurality of supports. In this specification and in the embodiments that follow, reference will be made to a number of terms that shall be defined to have the following meanings unless a contrary intention is apparent.

Prior to describing the various embodiments, the following definitions are provided and should be used unless otherwise indicated.

### DEFINITIONS

In describing the disclosed subject matter, the following terminology will be used in accordance with the definitions set forth below.

As used herein, the term “tomato” or “tomato plant” means any variety, cultivar, or population of Solanum lycopersicum (also known as Lycopersicon esculentum and/or Lycopersicon lycopersicum), including both commercial tomato plants as well as heirloom varieties. In some embodiments, “tomato” may also include wild tomato species, such as, but not limited to, Solanum lycopersicum var. cerasiforme, Solanum pimpineffifolium, Solanum cheesmaniae, Solanum neorickii, Solanum chmielewskii, Solanum habrochaites, Solanum pennellii, Solanum peruvianum, Solanum chilense and Solanum lycopersicoides.

As used herein, the term “plant” includes plant cells, plant protoplasts, plant cell tissue cultures from which tomato plants can be regenerated, plant calli, plant cell clumps, and plant cells that are intact in plants, or parts of plants, such as embryos, pollen, ovules, flowers, leaves, seeds, roots, root tips and the like.

The term “tomato fruit” refers to the fruit produced by a tomato plant, including the flesh, pulp, meat, and seeds of the fruit.

As used herein, the term “variety” or “cultivar” means a group of similar plants within a species that, by structural features, genetic traits, performance, and/or content of volatile compounds, sugars, and/or acids, can be identified from other varieties/cultrivars within the same species.

The term “volatile compound” refers to chemicals found in the fruit of the tomato plant that can be sensed by the olfactory systems of a consumer. Some exemplary volatile compounds include, but are not limited to, 1-penten-3-one, isovaleronitrile, trans-2-pentenal, trans-2-heptenal, trans-3-hexen-1-ol, 6-methyl-5-hepten-2-ol, nonyl aldehyde, cis-4-decenal, isovaleraldehyde, 3-methyl-1-butanol, methional, 2,5-dimethyl-hydroxy-3(2H)-furanone, 3-pentanone, 1-pentanol, benzyl cyanide, isovaleric acid, 2-isobutylthiazole, 1-nitro-3-methylbutane, benzaldehyde, 6-methyl-5-hepten-2-one, β-ionone, β-cyclocitral, geranial, phenylacetaldehyde, eugenol, geranylacetone, 2-phenylethanol, neral, salicylaldehyde, isobutyl acetate, butyl acetate, cis-3-hexen-1-ol, 1-nitro-2-phenylethane, 1-penten-3-ol, 2-methylbutyl acetate, heptaldehyde, trans,trans-2,4-decadienal, 2-methylbuteraldehyde, 4-carene, hexyl alcohol, guaiacol, propyl acetate, hexanal, cis-2-penten-1-ol, 2-butylacetate, 1-octen-3-one, cis-3-hexenal, methylsalicylate, trans-2-hexenal, β-damascenone, 2-methyl-1-butanol, 2-methyl-2-butenal, prenyl acetate, hexyl acetate, 3-methyl-1-pentanol, 2-ethylfuran, isopentyl acetate, benzothiazole, cis-3-hexenyl acetate, benzyl alcohol, citric acid, 3-methyl-2-butenal, 2-methylbutanal, and p-anisaldehyde.

The term “flavor-associated compound” refers to chemicals found in the fruit of the tomato plant that can be sensed by the taste and/or olfactory systems of a consumer that include, but are not limited to, volatile compounds, as discussed above, as well as various sugars and acids.

The term “heirloom tomato” lacks a specific definition in general usage, but generally refers to an open-pollinated variety of tomato that has been passed down through several generations, often maintained by a specific family. “Heirloom” also generally refers to a long-established variety of inbred tomatoes, usually from pre-1940 or in circulation for at least 50 years. Heirloom tomatoes are not usually hybrid, although, according to one definition, a category of heirloom tomatoes, the “created heirloom” results from a cross between two heirloom varieties, where the offspring variety then becomes an heirloom once it has been bred (usually through several generations) to a stable progeny line. Many heirloom varieties of tomato are “indeterminate,” indicating that the plant continues to produce fruit throughout the growing season as opposed to producing all fruit in a specific time frame (“determinate”). As used in the present disclosure, the term “heirloom,” “heirloom variety or cultivar,” or “heirloom tomato” refers to a tomato or tomato plant meeting any of the above criteria for “heirloom” status and that is not an “elite tomato/variety/cultivar” as defined below. In some embodiments, the “heirloom tomatoes” of the present disclosure, refer to open-pollinated, inbred (non-hybrid) varieties.

As used herein, the term “elite tomato,” “elite tomato plant” or “elite tomato variety or cultivar” refers to a tomato variety that has been cultivated and bred for performance and to have commercially desirable characteristics (e.g., suitable for mass production and marketing, a “supermarket tomato”). Elite tomatoes are used by breeders to create commercial tomato varieties. Commercial tomatoes are usually hybrids, produced by controlled pollination with elite tomatoes, which may involve artificial techniques (e.g., by hand, by machine, etc.) to control the pollination. Thus, “elite tomato variety” or “elite hybrid tomato parent” may refer to the parent of a hybrid commercial tomato or a tomato that is being bred to become a commercial tomato line. Elite tomatoes have been bred for characteristics such as fruit shape, color, hardiness, uniformity of size, disease resistance, uniformity of fruit set, and the like. Elite tomatoes may be determinate or indeterminate. A “commercial tomato” is a descendent of an “elite tomato” that has been commercialized (e.g., sold in commerce), though as used herein “commercial tomato” may also refer to a tomato variety that is being bred for commercial traits even if it has not yet been sold in commerce. As used herein the term “elite tomato” includes lines used in breeding commercial tomatoes as well as lines used in breeding tomatoes that are not yet commercialized. The intent is not to limit the disclosure to only to commercial varieties descended from elite tomatoes. For purposes of the present disclosure, “elite tomatoes” do not include “heirloom tomatoes” and vice versa.

As used herein, the term “hybrid” means any offspring (e.g., seed) produced from a cross between two genetically unlike individuals (Rieger, R., A Michaelis and M. M. Green, 1968, A Glossary of Genetics and Cytogenetics, Springer-Verlag, N.Y.). An “F1 hybrid” is the first generation offspring of such a cross, while an “F2”, “F3” hybrid, and so on, refer to descendent offspring from subsequent crosses (e.g., backcrossing of an F1 hybrid or later hybrid with one of the parent plant varieties, crossing an F1 hybrid with a different plant variety than the original parents, and so on). In some embodiments of the plants of this disclosure, an “F1 hybrid” refers to the offspring of a cross between an heirloom tomato, as one parent, and an elite tomato plant or parent of an elite tomato plant, as the other parent. In the present disclosure, the term “commercial hybrid” or “elite hybrid” is also used, which is distinguished from the “hybrid tomato” or “F1 hybrid” tomato of the present disclosure. The term “commercial hybrid” or “elite hybrid” as discussed above, refers to a commercial variety, which is usually a hybrid tomato, or elite parent of a commercial hybrid tomato, bred specifically for traits like disease resistance, growth, performance, and the like. (see the definition of “elite tomato” and “commercial tomato” above). The terms “elite tomato” and “elite hybrid” or “commercial tomato” and “commercial hybrid” may be used interchangeably in the present disclosure, although it is understood by those of skill in the art that not all commercially grown tomatoes are hybrids, and the intent is not to limit the present disclosure to discussion of hybrid commercial tomatoes.

The “hybrid tomato plants” and “hybrid tomatoes” of the present disclosure include descendants of an “elite tomato” and an “heirloom tomato”, meaning that such “hybrid tomatoes” have at least one heirloom tomato ancestor and at least one elite tomato ancestor. As used herein “ancestor” refers to a parent, grandparent, great-grandparent, and so-on, of a tomato plant. In an embodiment, a hybrid tomato plant of the present disclosure may be a descendent of an ancestor heirloom tomato and an ancestor elite tomato. In an embodiment of the present disclosure, an “F1 hybrid” is the direct offspring of a cross between a parent heirloom tomato and a parent elite tomato.

As used herein, the term “inbred” means a substantially homozygous plant or variety.

As used herein, the term “introgression” or “introgressed” means the entry or introduction of one or more genes from one or more plants into another. As used herein, the term “introgressing” means entering or introducing one or more genes from one or more donor or ancestor plants into a recipient or descendent. Introgression may be accomplished by either traditional breeding techniques or by transgenic methods, or a combination of genetic transformation and traditional breeding.

The term “tasting panel” refers to a number of individuals assembled into a panel to taste samples of tomatoes from different varieties and to rate the tomato samples based on flavor and other criteria. As used herein, the term “liking score” refers to a numerical score assigned to a sample tomato by a member of a tasting panel, where the taster rates the tomato based on the taster's perception of the taste of the tomato (e.g., liking or disliking).

As used herein, “positively associated with taste” or “positively associated with liking” indicates that a criterion (e.g., a volatile compound, other flavor associated compound, a ratio of flavor associated compounds or relative amounts, and the like) is correlated with a positive liking score, or a liking score that is above average. Thus, the terms “negatively associated with taste” or “negatively associated with liking” are used herein to indicate that a flavor criterion is correlated with a negative liking score, or a liking score that is below average.

As used herein, the phrase “better-tasting tomato” refers to a tomato with a better taste (e.g., improved liking), according to the average consumer, than a tomato from a standard elite or commercial variety (e.g., a supermarket tomato). In embodiments, a “better-tasting tomato” with reference to a hybrid tomato of the present disclosure, the better taste is relative to the taste of a fruit from an elite ancestor tomato variety. In embodiments, the better-taste is determined based on the “liking score” as defined herein from a “tasting panel”.

### Discussion

The embodiments of the present disclosure encompass hybrid tomatoes that produce better-tasting fruit than many mass-produced commercial varieties, methods of identifying the chemical composition of a tomato that leads to a better-tasting fruit, and methods of producing tomato varieties that produce better-tasting fruit. In embodiments, the present disclosure includes hybrid tomato varieties that produce fruit with greater amounts of certain flavor-associated compounds that positively correlate to liking/taste or lesser amounts of flavor-associated compounds that negatively correlate to liking/taste than an elite tomato ancestor. The present disclosure also includes methods of identifying tomatoes that produce better-tasting fruit by identifying flavor-associated compounds that positively and negatively associate with liking, and methods of producing new hybrid tomato varieties that produce better-tasting fruit and that have a greater amount of the compounds that positively associate with liking and/or a lesser amount of flavor-associated compounds that negatively associate with liking than a parent or ancestor elite tomato variety. Embodiments of the present disclosure also include new hybrid tomato varieties produced by crossing elite tomato varieties with heirloom tomato varieties and backcrossing offspring of such crosses to select for features from the heirloom tomato ancestor (such as better flavor due to optimized amounts of flavor-associated compounds) and features from the elite tomato ancestor (such as better field performance, better disease resistance, etc.). The methods of the present disclosure provide for the production of new tomato varieties with the commercially desirable features of an elite tomato and the flavor features of an heirloom tomato.

Tomato flavor is determined by complex interactions of a diverse set of flavor-associated compounds, which are chemicals that are sensed by the taste and olfactory systems. These chemicals include sugars (glucose and fructose), acids (citrate and malate) and a set of less well defined volatiles (4). The volatiles are synthesized via multiple independent metabolic pathways from amino acids, fatty acids and carotenoid precursors (5,6). The large number of independent metabolic pathways represents a major challenge to flavor quality improvement. Identification of the most important volatile contributors to flavor has been particularly difficult. An initial list of the important volatiles was assembled based on “odor units”, the ratio of concentration present in the fruit to the odor threshold for the pure compound (7). However, this approach can only be considered an approximation. Odor thresholds of pure compounds can be misleading. Olfactory receptors work in a combinatorial manner; a single odorant is recognized by multiple receptors while a single receptor recognizes multiple odorants (8). There is also cross-talk between the taste and olfactory systems with odorants influencing the magnitude of taste responses and vice versa (9). Thus, determining the chemical nature of a tomato with superior flavor will facilitate the production of such a tomato.

Example 1 below describes in greater detail embodiments of methods used to conduct a tasting panel according to the present disclosure and methods of identifying the flavor-associated compounds (e.g., sugars, acids, and volatile compounds) positively and negatively associated with liking and methods of identifying which tomato varieties have greater or lesser amounts of various volatile compounds and other flavor-associated compounds. Based on the data obtained from such studies, formulas can be determined, as described in Example 1, for identifying target amounts of various volatile compounds, sugars, and acids, or ratios thereof. As shown in Tables 2 and 3, the amounts of various flavor-associated compounds for tomatoes with different liking scores can be determined, with 34 representing the highest score given to any tomato actually tasted by the panel, 43 representing the score of the idealized best tomato ever tasted, and with 20 representing the score of a tomato with a generally acceptable liking level. This information provides guidelines for selecting tomato varieties for use in breeding programs to produce new hybrid tomato varieties with optimized levels of flavor-associated compounds, while still retaining some of the commercially desirable features of elite tomato varieties.

Embodiments of the present disclosure include methods of identifying hybrid tomato plants that produce better-tasting fruit. In such methods, first tomato samples from a plurality of different tomato plant varieties are provided to a tasting panel. The parameters of the tasting panel are controlled, such as described below in Example 1. Each member of the panel assigns a liking score to each tomato tasted (tested), and the results are accumulated. Also, a chemical analysis is performed on tomatoes from each of the variety of tomatoes tested by the panel. In the chemical analysis, each of a plurality of flavor-associated compounds from each tomato is quantified. The flavor-associate compounds can include, but are not limited to, sugars, acids, and volatile compounds. At least one of the flavor-associated compounds quantified is a volatile compound. The tasting panel scores are correlated to the calculated amounts of flavor-associated compounds for each tomato to determine which volatile compounds are positively associated with liking and which volatile compounds are negatively associated with liking. In embodiments, a formula and/or criteria associated with liking can be derived from this data. The formula indicates which volatile-compounds and/or other flavor-associated compounds, and/or what amounts of these compounds influence the general liking of a tomato. Determining criteria such as, but not limited to, volatile compounds positively associated with liking, volatile compounds negatively associated with liking, sugars and acids positively and negatively associated with liking, sugar: acid ratios positively associated and negatively associated with liking, and amounts of such compounds positively and/or negatively associated with liking.

In embodiments statistical analysis is conducted on tasting panel data, as described in Examples 1, 2, and 3 to determine some of the criteria (e.g., the identity of certain volatile compounds and/or content ranges of such compounds in a tomato fruit) that can be used to identify and/or select a tomato plant that produces better-tasting fruit as compared to an elite ancestor tomato or a standard supermarket tomato (e.g. a commercial variety). These criteria can then be used to select, identify, produce, and/or breed better tasting tomatoes. In embodiments formulas for better-tasting tomatoes can be determined from this information. For instance, parent heirloom tomatoes with desirable levels of volatile compounds positively or negatively associated with liking can be selected based on a chemical analysis of the volatile content of the fruit, and such tomatoes can be selected to breed with an elite line of tomatoes in order to produce a hybrid tomato with desirable characteristics of both the heirloom ancestor (e.g., improved taste/liking score) and the elite ancestor (e.g., improved texture and/or hardiness).

In embodiments, volatile compounds quantified include but are not limited to, 1-penten-3-one, trans-2-pentenal, trans-2-heptenal, trans-3-hexen-1-ol, trans-2-hexenal, cis-2-penten-1-ol, 6-methyl-5-hepten-2-ol, nonyl aldehyde, isovaleronitrile, cis-4-decenal, 3-methyl-1-butanol, 2,5-dimethyl-4-hydroxy-3(2H)-furanone, 1-pentanol, methional, benzyl cyanide, isovaleraldehyde, 3-pentanone, 2-isobutylthaizole, benzaldehyde, isovaleric acid, 1-nitro-3-methylbutane, β-ionone, β-cyclocitral, 6-methyl-5-hepten-2-one, geranial, phenylacetaldehyde, geranylacetone, 2-phenyl ethanol, 1-octen-3-one, eugenol, salicylaldehyde, isobutyl acetate, butyl acetate, 2-methylbutanal, and combinations of those compounds. In embodiments of methods of identifying hybrid tomato plants that produce better-tasting fruit of the present disclosure, the volatile compounds quantified include one or more of the above-listed compounds. In embodiments, the volatile compounds quantified include two or more, three or more, four or more, and so on of the above-listed volatile compounds.

In embodiments of the present disclosure, volatile compounds positively associated with liking include, but are not limited to, 1-penten-3-one, trans-2-pentenal, trans-2-heptenal, trans-3-hexen-1-ol, trans-2-hexenal, cis-2-penten-1-ol, 6-methyl-5-hepten-2-ol, nonyl aldehyde, isovaleronitrile, cis-4-decenal, 3-methyl-1-butanol, 2,5-dimethyl-4-hydroxy-3(2H)-furanone, 1-pentanol, methional, benzyl cyanide, isovaleraldehyde, 3-pentanone, 2-isobutylthiazole, benzaldehyde, isovaleric acid, 1-nitro-3-methylbutane, β-ionone, β-cyclocitral, 6-methyl-5-hepten-2-one, geranial, phenylacetaldehyde, geranylacetone, 2-phenyl ethanol, or 1-octen-3-one, or any combination of these compounds. In some embodiments, volatile compounds positively associated with liking are chosen from 1-penten-3-one, trans-2-hexenal, cis-2-penten-1-ol, geranial, 3-methyl-1-butanol, 1-octen-3-one, trans-2-pentenal, isovaleronitrile, trans-3-hexen-1-ol, 1-nitro-3-methylbutane, or 6-methyl-5-hepten-2-one, or any combination of two or more of these volatile compounds. In embodiments of the present disclosure tomato fruit of the tomato plants of the present disclosure include a greater amount (e.g., than an ancestor elite tomato plant) of one or more of the above-listed compounds, two or more, three or more, four or more, and so on of the above-listed volatile compounds.

In embodiments of the present disclosure, volatile compounds negatively associated with liking include, but are not limited to, eugenol, salicylaldehyde, isobutyl acetate, butyl acetate, 2-methylbutanal or combinations of those compounds. In embodiments of the present disclosure tomato fruit of the tomato plants of the present disclosure include a lower amount (e.g., than a fruit produced by an ancestor elite tomato plant) of one or more of the above-listed compounds, two or more, three or more, four or more, and so on of the above-listed volatile compounds. In embodiments of the present disclosure, tomato fruits produced by tomato plants of the present disclosure may have any combination of a greater amount of one or more, two or more, three or more, and so on, of volatile compounds positively associated with liking and also have a lower amount (e.g., than a fruit produced by an ancestor elite tomato plant) of one or more, two or more, three or more, and so on of volatile compounds negatively associated with liking. Thus, the present disclosure includes tomatoes having any combination of a greater amount of volatiles positively associated with liking and/or a lesser amount of volatiles negatively associated with liking than a comparative tomato (e.g., an ancestor elite tomato fruit).

The present disclosure includes additional embodiments of methods of identifying a better tasting tomato fruit by performing a chemical analysis of a tomato fruit from each of a variety of tomato plants and selecting tomato fruit having the greatest amount of one or more compound positively associated with liking and/or the least amount of one or more compounds negatively associated with liking. The volatile compounds positively and negatively associated with liking are set forth above. In embodiments, the compounds positively associated with liking are chosen from compounds 1-penten-3-one, trans-2-hexenal, cis-2-penten-1-ol, geranial, 3-methyl-1-butanol, 1-octen-3-one, trans-2-pentenal, isovaleronitrile, trans-3-hexen-1-ol, 1-nitro-3-methylbutane, 6-methyl-5-hepten-2-one, 2-methylbutanal, butyl acetate, isobutylacetate, and eugenol, and combinations thereof, and the compounds negatively associated with liking are chosen from compounds 2-methylbutanal, butyl acetate, isobutylacetate, and eugenol.

For instance, in embodiments of the present disclosure, a hybrid tomato of the present disclosure would have an amount of at least one of the volatile compounds positively associated with liking that is at least the amount found in a tomato having a liking score of 20, as illustrated in Table 3. In other embodiments, a hybrid tomato of the present disclosure would have an amount of at least one of the volatile compounds negatively associated with liking that is less than the amount found in a tomato having a liking score of 20. In other embodiments, a hybrid tomato of the present disclosure would have a greater amount of at least one of the volatile compounds positively associated with liking and/or a lesser amount of at least one of the volatile compounds negatively associated with liking than an ancestor elite tomato. In embodiments, a hybrid tomato of the present disclosure has a greater amount of a combination of one or more of the volatile compounds positively associated with liking and/or a lesser amount of one or more of the volatile compound negatively associated with liking than an ancestor elite tomato.

In embodiments, the volatile compound positively associated with liking can be, but is not limited to, 1-penten-3-one, trans-2-pentenal, trans-2-heptenal, trans-3-hexen-1-ol, 6-methyl-5-hepten-2-ol, nonyl aldehyde, isovaleronitrile, cis-4-decenal, 3-methyl-1-butanol, 2,5-dimethyl-4-hydroxy-3(2H)-furanone, 1-pentanol, methional, benzyl cyanide, isovaleraldehyde, 3-pentanone, 2-isobutylthaizole, benzaldehyde, isovaleric acid, 1-nitro-3-methylbutane, β-ionone, β-cyclocitral, 6-methyl-5-hepten-2-one, geranial, phenylacetaldehyde, geranylacetone, or 2-phenyl ethanol, or combinations of those compounds. In some embodiments, the volatile compound positively associated with liking is one or more of the following: 1-penten-3-one, trans-2-hexenal, cis-2-penten-1-ol, geranial, 3-methyl-1-butanol, 1-octen-3-one, trans-2-pentenal, isovaleronitrile, trans-3-hexen-1-ol, 1-nitro-3-methylbutane, 6-methyl-5-hepten-2-one.

In embodiments, the volatile compound negatively associated with liking can be, but is not limited to, eugenol, salicylaldehyde, isobutyl acetate, butyl acetate, 2-methylbutanal or combinations of those compounds. In embodiments, the volatile compound negatively associated with liking is one or more of isobutyl acetate, butyl acetate, and 2-methylbutanal.

In some embodiments, some of the volatile compounds positively associated with taste liking are also positively associated with aroma liking. Such compounds include, but are not limited to, 1-penten-3-one, trans-2-hexanal, and cis-2-penten-1-ol, or a combination thereof.

In embodiments, other flavor-associated compounds that are analyzed for association with liking score, include, but are not limited to, sugar content (e.g., glucose, fructose, and a combination of both sugars) and acid content (e.g., malic acid, citric acid, etc.). In some embodiments, ratios of sugar:acid and citrate:malate are also considered. In embodiments, heirloom tomatoes used in methods of producing new hybrids and/or the hybrid tomatoes of the present disclosure have higher sugar content than the fruit of an elite tomato variety that is a parent or ancestor of a hybrid tomato of the present disclosure. In embodiments, the sugar:acid ratio of such heirloom tomatoes and/or the hybrid tomatoes of the present disclosure is from about 8 to about 16.

Using the information obtained from the methods of identifying flavor-associated compounds that positively and negatively affect taste (e.g., as determined by liking score) of a tomato described in the present disclosure, traditional breeding techniques can be used to generate new hybrid tomato varieties. For instance, information obtained from analyzing volatile compounds and tasting panel scores, assists in selection of tomato varieties to use in breeding programs to produce new hybrid tomato varieties with improved flavor ratings. In embodiments, an heirloom tomato with a high liking score, relative to a another heirloom or commercial tomato tested, can be chosen for crossing with an elite tomato variety or an elite parent of a hybrid commercial tomato variety (e.g., a commercial tomato, or a non-commercialized elite tomato variety) to produce a hybrid tomato with improved flavor over the elite parent variety and/or the hybrid commercial tomato variety. In embodiments, the heirloom tomato variety used in the cross can be an heirloom variety with an overall liking score of at least 20. In embodiments, the heirloom tomato variety used in the cross can be, but is not limited to, Cherry Roma, Matina, Ailsa Craig, Red Calabash, Red Pear, Bloody Butcher, Maglia Rosa Cherry, Brandywine, Tommy Toe, Chadwick Cherry, Livingston's Stone, Super Sioux, St. Pierre, German Queen, Wisconsin 55, Micado Violettor, Livingston's Globe, and Gulf State Market.

In embodiments, the heirloom tomato variety selected for crossing has a greater amount of at least one of the volatile compounds positively associated with liking and/or a lesser amount of at least one of the volatile compounds negatively associated with liking than a fruit of the elite tomato variety selected for the cross. In embodiments, the hybrid tomato produced from the cross (e.g., the F1 hybrid, or subsequent hybrids produced by downstream crosses) produces a fruit with a greater amount of at least one of the volatile compounds positively associated with liking and/or a lesser amount of at least one of the volatile compounds negatively associated with liking than a fruit of the ancestor elite tomato variety selected for the initial cross (e.g., the ancestor elite tomato). As noted above, in embodiments, the hybrid tomato produced from the cross produces a fruit with a greater amount of at least one, at least two, at least three, and so on up to a greater amount of all of the listed volatile compounds positively associated with liking than the ancestor elite tomato. Similarly, the hybrid tomato produced from the cross in embodiments has a lower amount of at least one, at least two, at least three, and so on up to a lesser amount of five or more of the volatile compounds negatively associated with liking than the ancestor elite tomato.

Embodiments of hybrid tomatoes of the present disclosure include hybrid tomato plants that produce tomato fruit having a greater amount of at least one volatile compound positively associated with liking in the fruit of the hybrid plant than the amount of that volatile compound in in fruit produced by an ancestor elite tomato cultivar. In embodiments, the volatile compound is chosen from: 1-penten-3-one, trans-2-pentenal, trans-2-heptenal, trans-3-hexen-1-ol, trans-2-hexenal, cis-2-penten-1-ol, 6-methyl-5-hepten-2-ol, nonyl aldehyde, isovaleronitrile, cis-4-decenal, 3-methyl-1-butanol, 2,5-dimethyl-4-hydroxy-3(2H)-furanone, 1-pentanol, methional, benzyl cyanide, isovaleraldehyde, 3-pentanone, 2-isobutylthaizole, benzaldehyde, isovaleric acid, 1-nitro-3-methylbutane, β-ionone, β-cyclocitral, 6-methyl-5-hepten-2-one, geranial, phenylacetaldehyde, geranylacetone, 2-phenyl ethanol, 1-octen-3-one, or combinations thereof. In embodiments, the volatile compound is chosen from compounds: 1-penten-3-one, trans-2-hexenal, cis-2-penten-1-ol, geranial, 3-methyl-1-butanol, 1-octen-3-one, trans-2-pentenal, isovaleronitrile, trans-3-hexen-1-ol, 1-nitro-3-methylbutane, 6-methyl-5-hepten-2-one, and combinations thereof. In various embodiments of the present disclosure, the hybrid tomato plants of the present disclosure produce tomato fruit having a greater amount of at least two volatile compounds, or at least three volatile compounds, or at least 4 volatile compounds, or at least 5 volatile compounds, and so on up to a greater amount of all of the above-listed volatile compounds than a fruit produced by an ancestor elite tomato cultivar.

In some embodiments, a hybrid tomato plant of the present disclosure has one or more of the following volatile compounds in the following amounts, measured as volatile emission (ng of volatile emitted by 1 g of fresh weight tomato per hour (ng gFW⁻¹h⁻¹)): 1-penten-3-one, having a content of about 1.6 ng gFW⁻¹h⁻¹ or more; trans-2-pentenal, having a content of about 1.1 ng gFW⁻¹h⁻¹ or more; trans-2-heptenal, having a content of about 0.44 ng gFW⁻¹h⁻¹ or more; trans-3-hexen-1-ol, having a content of about 1.2 ng gFW⁻¹h⁻¹ or more; 6-methyl-5-hepten-2-ol having a content of about 0.17 ng gFW⁻¹h⁻¹ or more; nonyl aldehyde, having a content of about 0.30 ng gFW⁻¹h⁻¹ or more; isovaleronitrile, having a content of about 14.0 ng gFW⁻¹h⁻¹ or more; cis-4-decenal, having a content of about 1.2 ng gFW⁻¹h⁻¹ or more; 3-methyl-1-butanol, having a content of about 39.1 ng gFW⁻¹h⁻¹ or more; 2,5-dimethyl-4-hydroxy-3(2H)-furanone, having a content of about 0.39 ng gFW⁻¹h⁻¹ or more; 1-pentanol, having a content of about 3.8 ng gFW⁻¹h⁻¹ or more; methion al, having a content of about 0.16 ng gFW⁻¹h⁻¹ or more; benzyl cyanide, having a content of about 0.29 ng gFW⁻¹h⁻¹ or more; isovaleraldehyde, having a content of about 14.1 ng gFW⁻¹h⁻¹ or more; 3-pentanone, having a content of about 6.6 ng gFW⁻¹h⁻¹ or more; 2-isobutylthaizole, having a content of about 5.9 ng gFW⁻¹h⁻¹ or more; benzaldehyde, having a content of about 3.1 ng gFW⁻¹h⁻¹ or more; isovaleric acid, having a content of about 0.08 ng gFW⁻¹h⁻¹ or more; 1-nitro-3-methylbutane, having a content of about 19.1 ng gFW⁻¹h⁻¹ or more; β-ionone, having a content of about 0.06 ng gFW⁻¹h⁻¹ or more; β-cyclocitral, having a content of about 0.10 ng gFW⁻¹h⁻¹ or more; 6-methyl-5-hepten-2-one, having a content of about 4.2 ng gFW⁻¹h⁻¹ or more; geranial, having a content of about 0.15 ng gFW⁻¹h⁻¹ or more¹; phenylacetaldehyde, having a content of about 0.29 ng gFW⁻¹h⁻¹ or more; geranylacetone, having a content of about 1.61 ng gFW⁻¹h⁻¹ or more; 2-phenyl ethanol, having a content of about 0.7 ng gFW⁻¹h⁻¹ or more; 1-octen-3-one, having a content of about 0.06 ng gFW⁻¹h⁻¹ or more; trans-2-hexenal, having a content of about 4.6 ng gFW⁻¹h⁻¹ or more; and cis-2-penten-1-ol, having a content of about 1.2 ng gFW⁻¹h⁻¹ or more.

Embodiments of hybrid tomatoes of the present disclosure also include hybrid tomato plants that produce tomato fruit having a lesser amount of at least one volatile compound negatively associated with liking in the fruit of the hybrid plant than the amount of that volatile compound in in fruit produced by an ancestor elite tomato cultivar. In embodiments, the volatile compound negatively associated with liking is chosen from eugenol, salicylaldehyde, isobutyl acetate, butyl acetate, 2-methylbutanal, or combinations thereof. In some embodiments, the volatile compound negatively associated with liking is chosen from one or a combination of the following compounds: isobutyl acetate, butyl acetate, and 2-methylbutanal. In embodiments, one or more of the compounds negatively associated with liking are present in the hybrid tomato of the present disclosure in the following amounts: about 0.48 ng gFW⁻¹h⁻¹ or less of eugenol, about 3.9 ng gFW⁻¹h⁻¹ or less of 2-methylbutanal, about 0.17 ng gFW⁻¹h⁻¹ or less of butylacetate, and about 0.95 ng gFW⁻¹h⁻¹ or less of isobutylacetate.

In further embodiments of the present disclosure tomatoes of the present disclosure, such as hybrid tomatoes of the present disclosure, have one or more of the volatile compounds positively associated with liking in a greater amount than tomatoes from an ancestor elite tomato cultivar and also have one or more of the volatile compounds negatively associated with liking in a lesser amount than tomatoes from an ancestor elite tomato cultivar. As discussed above, in additional embodiments of the present disclosure, tomatoes of the present disclosure have tow or more, three or more, or four or more, and so on of the volatile compounds positively associated with liking in a greater amount than tomatoes from an ancestor elite tomato cultivar and/or also have one or more, two or more, three or more, or four or more, and so on of the volatile compounds negatively associated with liking in a lesser amount than tomatoes from an ancestor elite tomato cultivar.

Additional embodiments of the present disclosure include methods of making a hybrid tomato plant of the present disclosure by crossing a parent of an elite hybrid tomato cultivar with an heirloom tomato cultivar, where the heirloom tomato cultivar produces tomato fruit with a greater amount of at least one volatile compound positively associated with liking than the elite hybrid tomato cultivar. This method can produce an F1 hybrid tomato plant that produces tomato fruit with a greater amount of the at least one volatile compound positively associated with liking than the elite hybrid tomato cultivar. Such methods may also include using an heirloom tomato cultivar that also produces tomato fruit with a lower amount of at least one volatile compound negatively associated with liking than the amount of that compound in the elite hybrid tomato cultivar. Thus, the F1 hybrid from such cross can produce fruit with a lower amount of the at least one volatile compound negatively associate with liking than in the ancestor elite hybrid cultivar.

In embodiments, an F1 hybrid tomato of the present disclosure, produced by crossing an heirloom tomato cultivar and a parent of an elite hybrid tomato cultivar, is further backcrossed with one of the parent cultivars to produce a subsequent hybrid tomato. In embodiments, the F1 hybrid is backcrossed with the elite tomato cultivar parent, and the offspring are selected for the flavor/volatile traits of the heirloom tomato cultivar ancestor. In other embodiments, the F1 hybrid is backcrossed with the heirloom tomato cultivar parent, and offspring are selected for the agricultural/commercial traits of the elite cultivar ancestor and the flavor components of the heirloom ancestor. In other embodiments, the hybrid tomato cultivars may be backcrossed through many generations, with the recurrent parent being the elite tomato cultivar to further improve the agricultural/commercial traits while still selecting for the flavor traits of the heirloom cultivar ancestor (the “donor line”). In other embodiments, the hybrid tomato cultivars may be backcrossed through many generations, with the recurrent parent being the heirloom tomato cultivar to further improve the flavor traits while still selecting for the agricultural traits of the elite cultivar ancestor (the “donor line”). In such embodiments, the progeny hybrid tomato cultivar can retain most of the genetic material of the recurrent parent but will also include desirable traits (that have been selected for) from the donor line. In embodiments, such backcrossing results in the introgression of the selected traits from the donor line. Thus, in embodiments, the present disclosure includes a hybrid tomato with genetic traits associated with levels of flavor-associated compounds from an heirloom tomato cultivar introgressed into the gene pool of the hybrid tomato.

Additional embodiments of the present disclosure include a hybrid descendent of an ancestor heirloom tomato cultivar and an ancestor elite cultivar, where the hybrid tomato plant produces tomato fruit with a greater amount of the at least one volatile compound positively associated with liking than the amount of that volatile compound present in fruit produced by the ancestor elite cultivar. Embodiments also include backcrossing a hybrid descendent of an ancestor heirloom tomato cultivar and an ancestor elite cultivar with one of the ancestor cultivars to produce a hybrid that produces fruit having a higher amount of at least one volatile compound positively associated with liking than the ancestor elite cultivar. In such embodiments the hybrid tomato plant may also be selected such as to produce fruit that has a lower amount of at least one volatile compound negatively associated with liking than the amount of that compound in the fruit of the ancestor elite hybrid tomato cultivar.

Additional details regarding the tasting panels, the chemical composition of the tomato cultivars, the analysis of chemical composition and liking scores, the hybrid tomatoes of the present disclosure and methods of making the hybrid tomatoes of the present disclosure can be found in the Examples below.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and protected by the following embodiments.

The following examples are put forth so as to provide those of ordinary skill in the art with a complete disclosure and description of how to perform the methods and use the compositions and compounds disclosed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Example 1

It is widely recognized that the flavor quality of many commercially produced fresh fruits has declined in recent years. Despite narrow genetic variation within the tomato species, there is a surprisingly large range of flavor chemicals. Knowing how the components of tomato flavor co-vary with human preference, and creating a system to ‘engineer’ these preferences constitutes a new direction in the chemistry of human flavor preferences for the tomato fruit specifically, and naturally grown food products in general. Eighty tomato varieties spanning the range of biochemical diversity were tested by consumers to generate a subjective sensory profile of perceptions, including overall liking. A total of 44 sugars, acids and volatiles were either significantly positively or negatively correlated with overall liking; many of the positively correlated volatiles were not previously associated with tomato flavor. Conversely, several volatiles widely accepted as being important contributors to flavor did not correlate with liking. The lack of correlation for the highly abundant C6 volatiles was tested and validated with transgenic fruits that do not synthesize these volatiles. Finally, regression analysis and reverse engineering created a model, identifying target levels of each flavor chemical, essentially defining the formula for an ideal tomato. This synthetic approach to understanding the chemistry of liking for complex natural products provides breeders with the knowledge to achieve flavor improvements. Such a synthetic approach establishes a formalized method for improving a complex natural food.

There are numerous as-yet undefined genes that influence flavor quality (1-3). The present example demonstrates a systematic approach to defining the chemical composition of a great-tasting tomato by exploiting the unexpectedly large chemical variation found within the species. This study identifies important chemicals contributing to good flavor, defining targets for molecular breeding. This integration of chemical and sensory science serves as a roadmap for quality improvement in fruit and vegetable crops.

As a first step toward establishing a molecular blueprint of tomato flavor, a chemical analysis on 278 samples was performed, representing 152 unique varieties of heirloom tomatoes. These varieties are mostly inbred, covering the range of available fruit color, shape and size. Most predate intensive breeding of the modern tomato cultivars (10). Levels of glucose, fructose, citrate, malate, glutamate, and 29 potential flavor volatiles were determined in all of the lines, most over multiple seasons (data not shown). Molecular studies indicate that there is a relatively low rate of DNA sequence diversity within the cultivated tomato, Solanum lycopersicum; the rate of single nucleotide polymorphism within translated sequences has been estimated at 0.1% with half of that being in the third codon position (11). This low rate of DNA polymorphism is consistent with the concept of a genetic bottleneck associated with two periods of domestication in central America and Europe (10). Despite the lack of sequence differences among the varieties, there is large variation in fruit size, shape and color. Nonetheless, it was surprising to observe variation in volatile contents of as much as three thousand-fold across the cultivars (Table 4). The molecular basis for this large variation has yet to be determined.

Seasonal variations in the absolute levels of volatiles produced by each cultivar were observed. However, the relative levels of each volatile within and between cultivars were consistent across seasons. For example, the same cultivars consistently had the highest and lowest amounts of cis-3-hexenal in each season. Thus, the biochemical differences observed were highly heritable but environmentally modulated. To further understand the phenotypic variation within the species, a set of 18 wild-collected S. lycopersicum var. cerasiforme accessions was grown in a single season in a greenhouse and volatiles were analyzed (data not shown). There was almost as much variation observed within this set of accessions as was observed within the heirloom set over multiple seasons, validating the surprising degree of chemical diversity within the species.

This large chemical diversity within the heirloom population offered an unprecedented opportunity to examine the individual contributions of the flavor-associated compounds (e.g., sugars, acids and volatiles) to flavor. This goal was accomplished by conducting sensory analysis with a consumer panel on a subset of the cultivars exhibiting broad chemical diversity. Panelists rated their overall liking and liking for the texture of each of the tomatoes as well as how much they liked the best and worst tomatoes they had ever tasted (e.g., the “best” and “worst” they had ever tasted in their lives, according to their memory, not necessarily a tomato tested on the consumer panel) on the hedonic general Labeled Magnitude Scale (hedonic gLMS) (12, 13). Panelists then rated the perceived intensities of overall tomato flavor, sweetness, sourness, saltiness and umami as well as the intensities they desired in their “ideal” tomato using the gLMS. Thirteen panels were conducted over three seasons. Sixty-six different cultivars as well as several varieties purchased at local supermarkets (e.g., representing commercial or elite tomato varieties) were evaluated by the panels. Several cultivars were repeated over multiple seasons. In each case, random samples of each set were removed for chemical analysis. The set of measured volatiles for the consumer panels was expanded to 61. Glucose, fructose, citrate, malate and glutamate levels as well as total soluble solids and citrate:malate and sugar:acid ratios were also determined (Table 8). The seasonal variations within some cultivars provided additional chemical diversity.

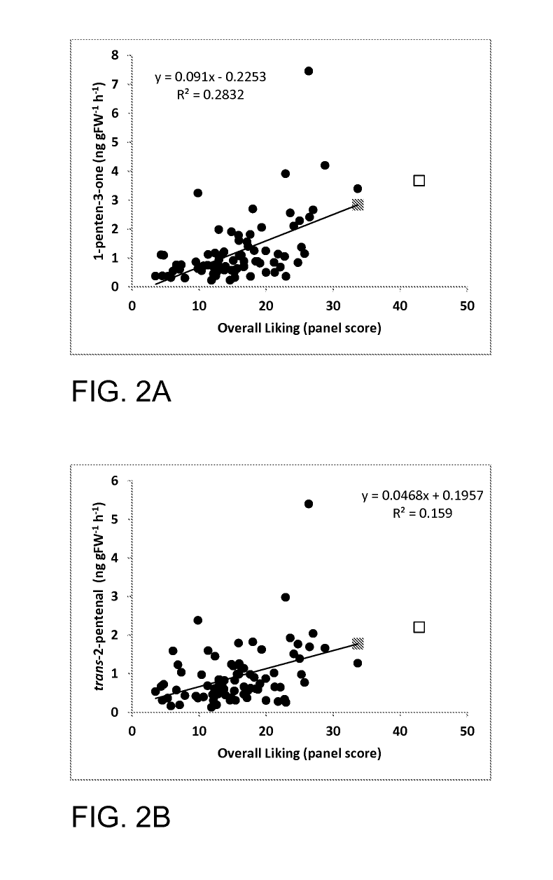

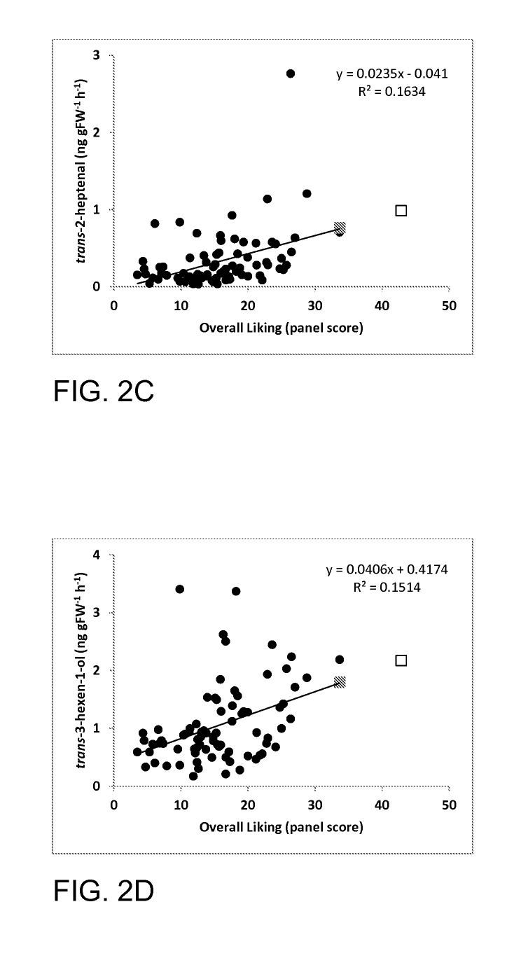

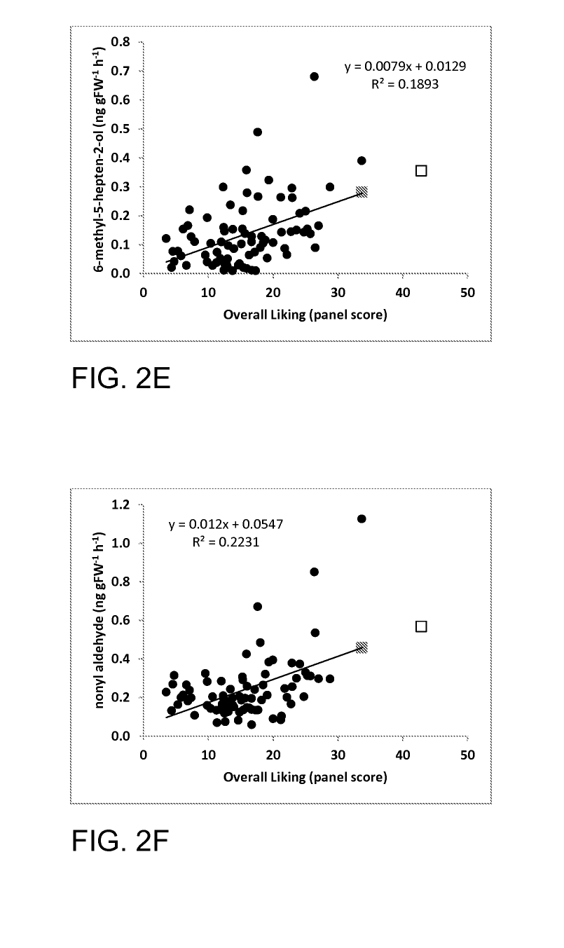

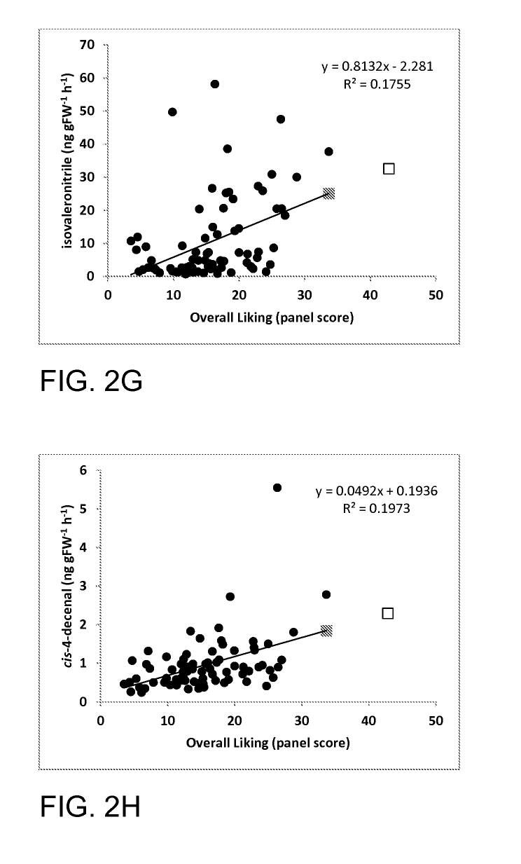

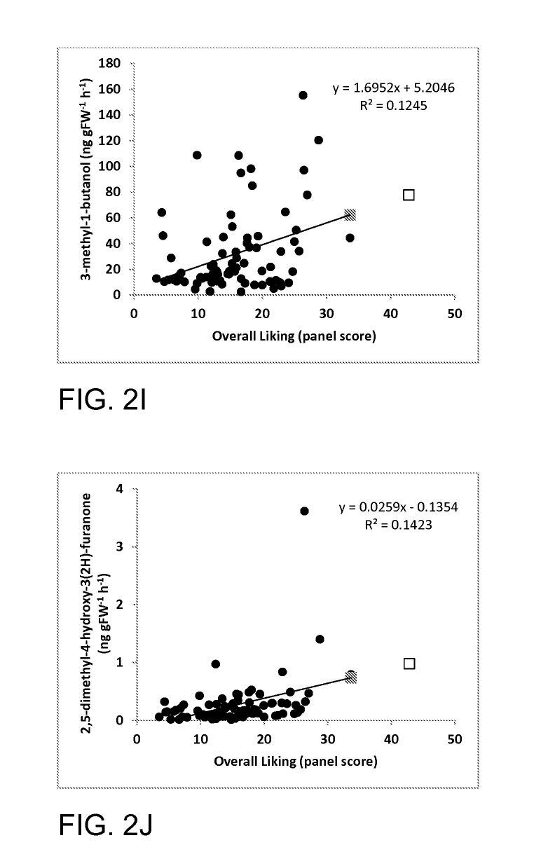

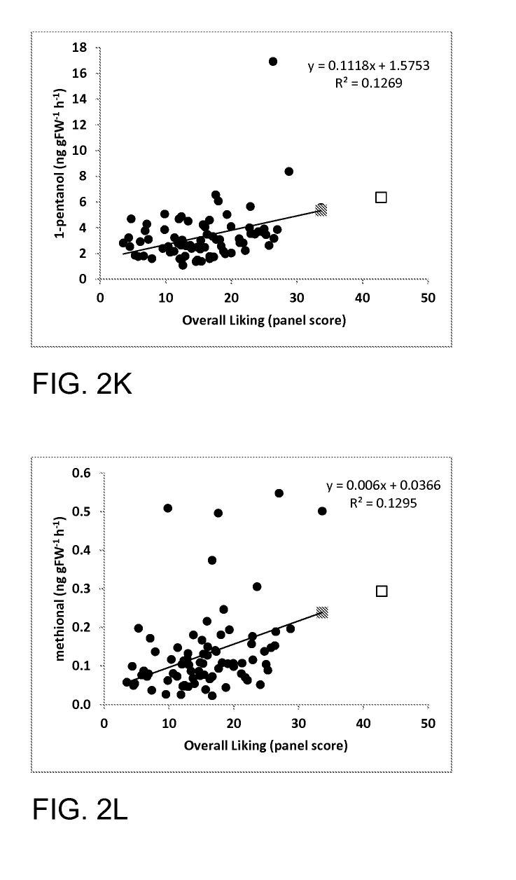

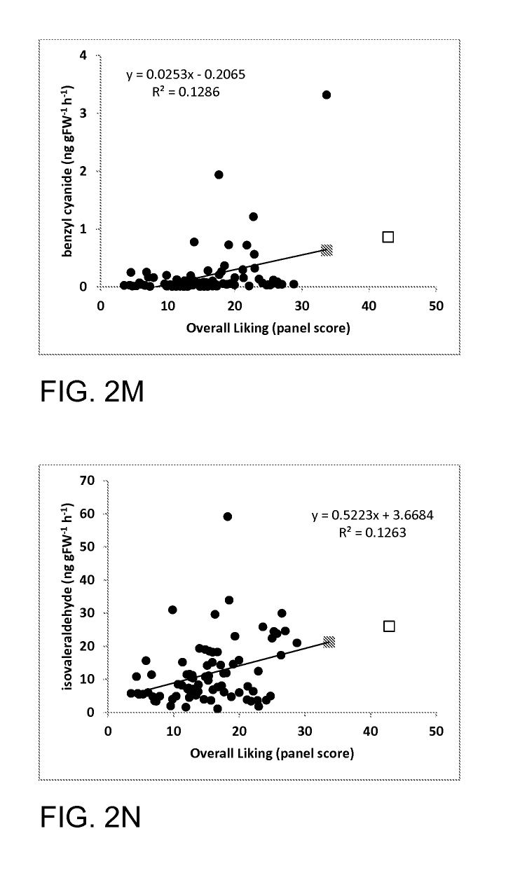

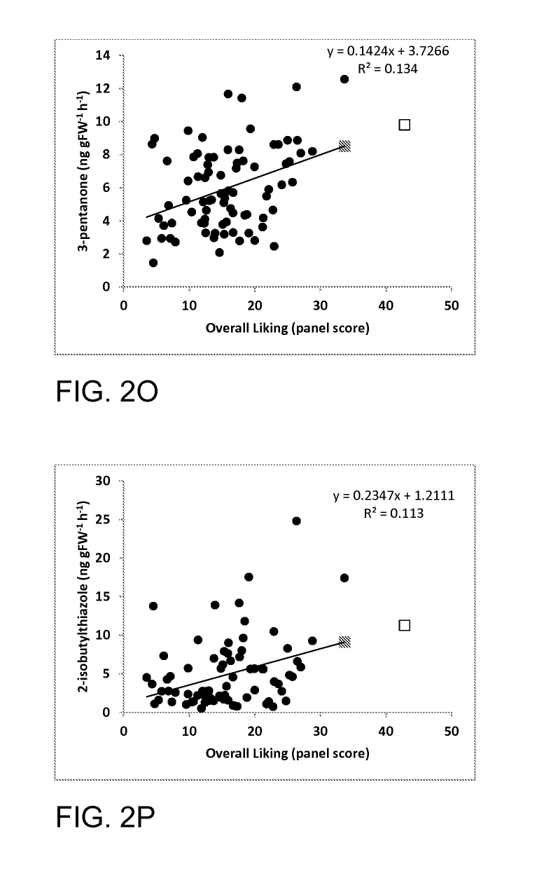

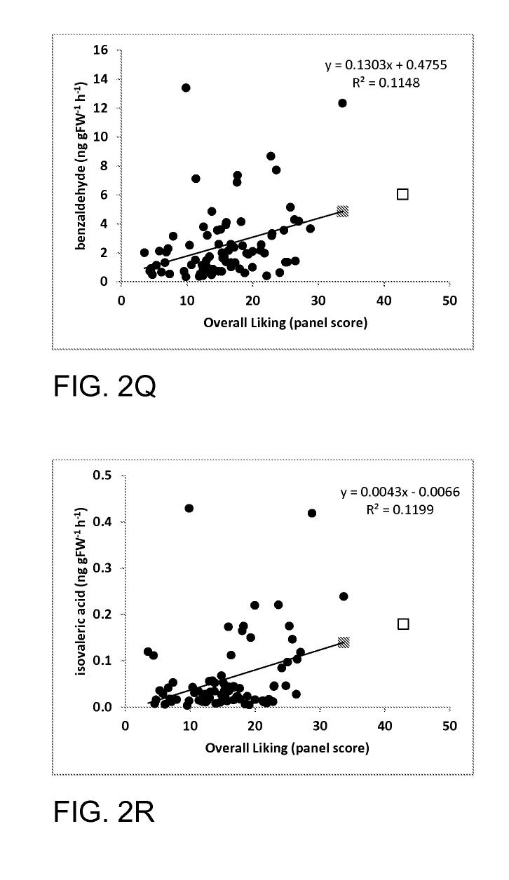

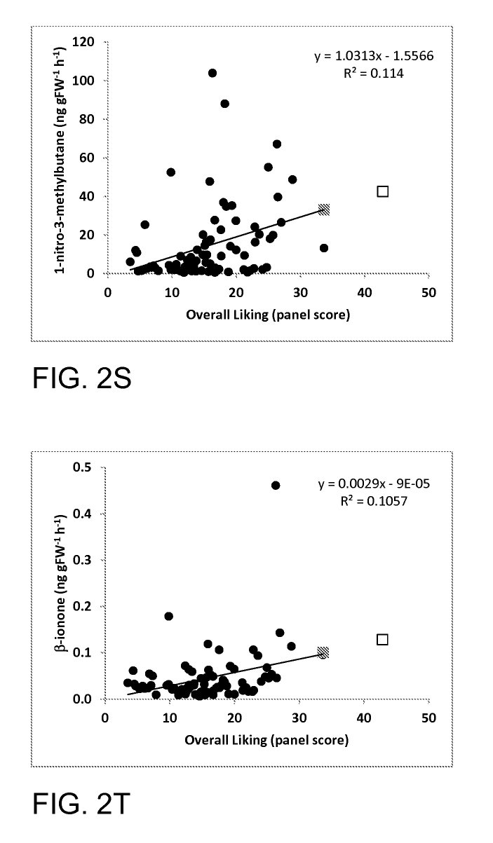

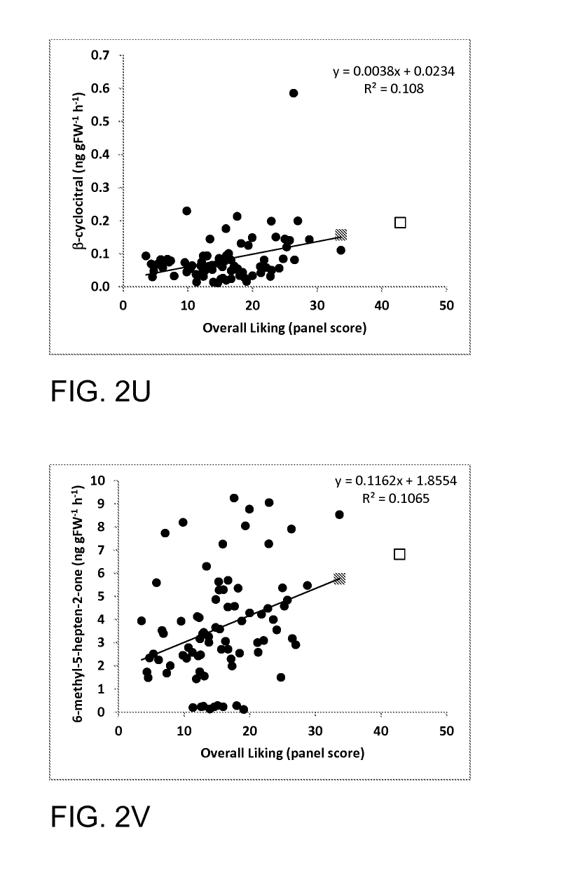

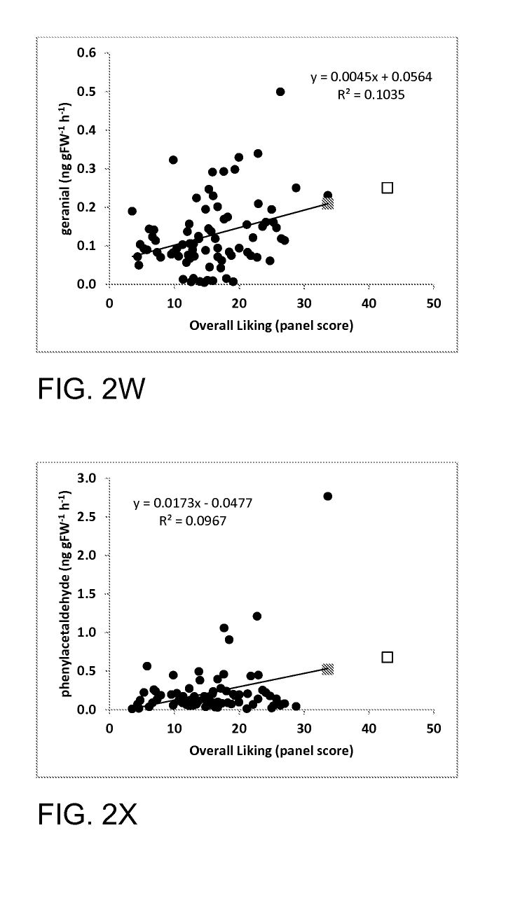

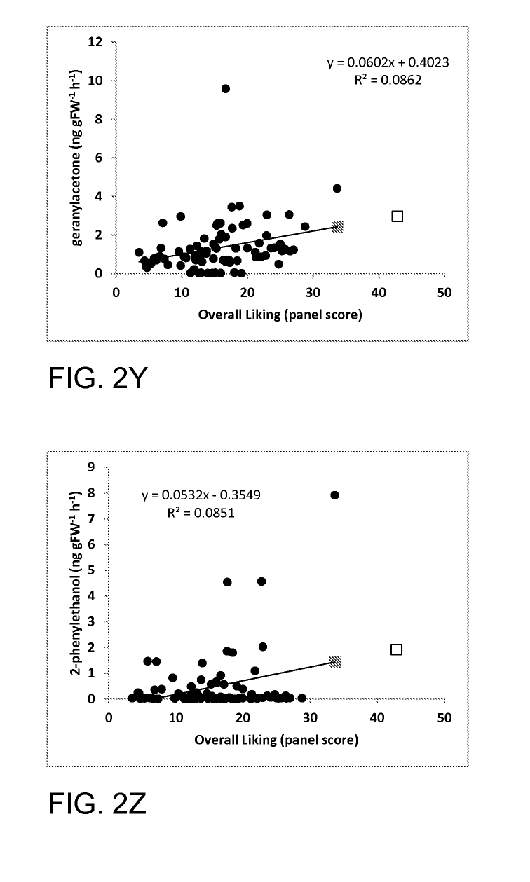

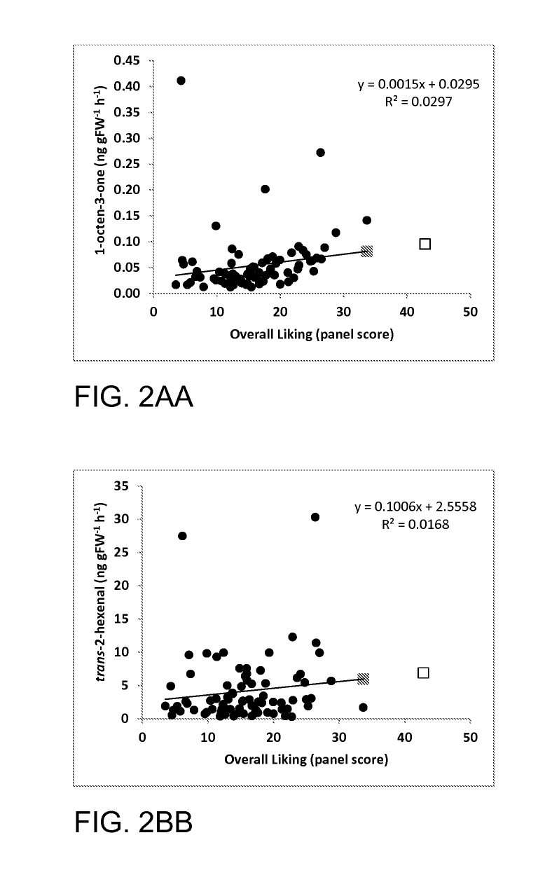

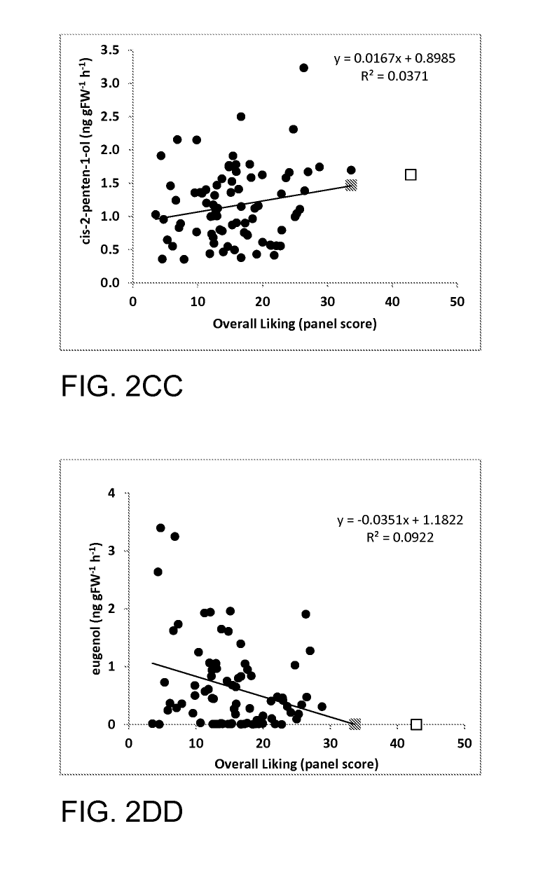

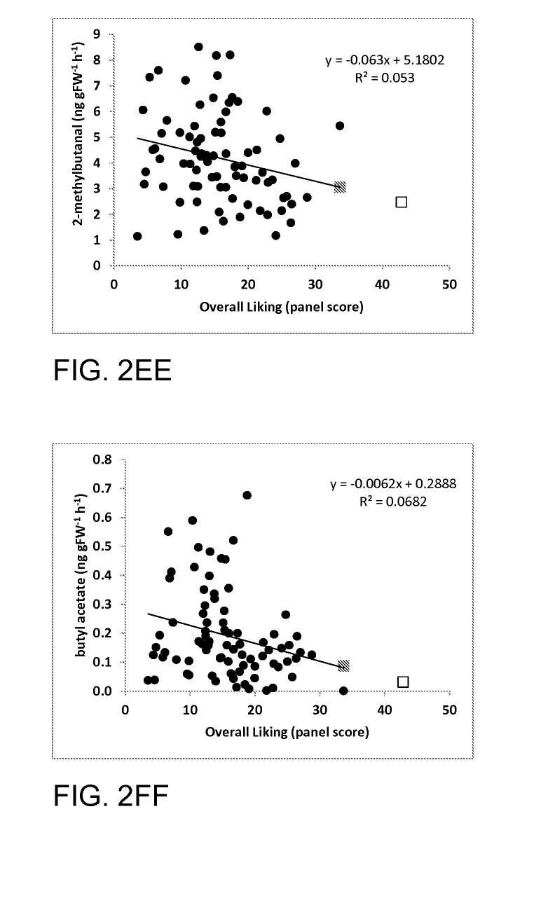

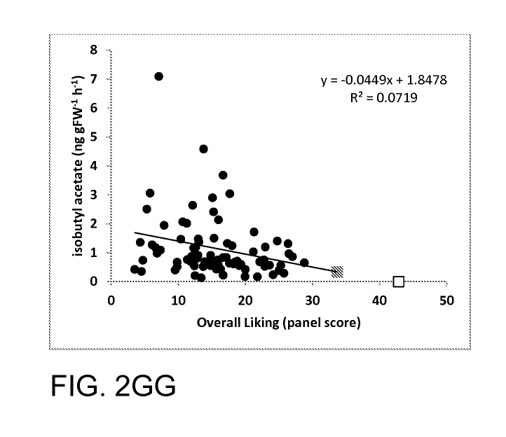

Linear regression modeling permitted identification of the chemicals that significantly impact liking. (FIGS. 2A-2GG, Table 1) At p<0.01, 26 volatiles as well as glucose and fructose were significantly correlated with liking. Twenty-three of the 26 volatiles were positively correlated while three were negatively correlated with liking. At p<0.05, an additional 12 volatiles were positively correlated while one volatile and malic acid were negatively correlated with liking (Table 1). Sugar content was an important predictor of liking. Acids were not tightly linked to liking, with the exception of malate, which was negatively correlated (p=0.037). Recently a negative correlation between malate and starch synthesis in tomato fruits was reported (14). Our data indicate a strong negative correlation between glucose and malate content (p=0.005). Glutamate, which is linked to umami, was not significantly correlated with liking in this study. Correlations between volatile content and the perception of overall tomato flavor intensity were also determined. These correlations were similar to those for liking (Table 1).

The contributions of the various volatile compounds to liking were unexpected. Prior lists of important tomato flavor volatiles were compiled based on the calculation of an “odor unit value,” which is defined to be “the ratio of the concentration of the component in the food to its odor threshold in water” (7). This definition does not take into account variation in the manner in which the perceived intensities of odorants grow with concentration. Two odorants can have the same “odor unit value” but produce olfactory sensations that are dramatically different in perceived intensity. Thus, the volatiles in tomatoes that contribute significantly to their flavor (and to liking) are not necessarily those with the highest “odor unit values.”

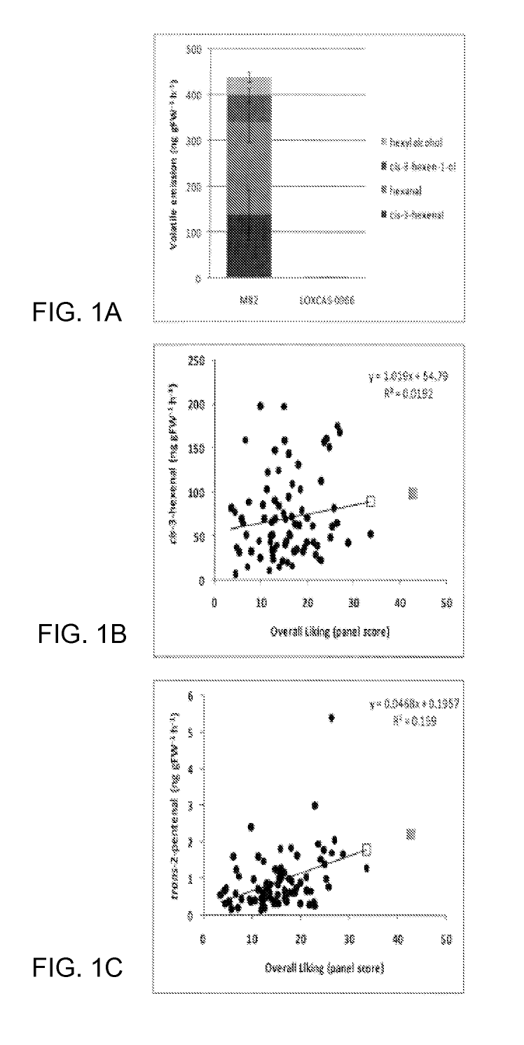

Multiple volatiles, such as trans-2-pentenal, have low “odor unit values” but significantly correlate with liking and tomato flavor (FIG. 1, Table 1). Conversely, volatiles such as cis-3-hexenal, with high “odor unit values” were not as highly correlated with liking (FIG. 1, Table 1). The unexpected observation that some of the most abundant volatiles in a fruit are not actually significant drivers of liking was directly tested. To validate the apparent minimal contribution of the C6 volatiles to liking, transgenic plants were used. The C6 volatiles (e.g., cis-3-hexenal, hexanal, cis-3-hexen-1-ol and hexyl alcohol) are synthesized from 18:2 and 18:3 fatty acids via the action of lipoxygenase (15). Antisense lines that do not express the enzyme responsible for their synthesis, LoxC (FIG. 1, Table 5) were produced. Although consumers were able to distinguish the transgenic from control fruits (p=0.009) via a triangle test for differences, there was no significant difference in preference between the two. Taken together, the results provide an entirely new insight into tomato flavor. Previous concepts of the most important volatile contributors to flavor based on odor thresholds must be reevaluated.

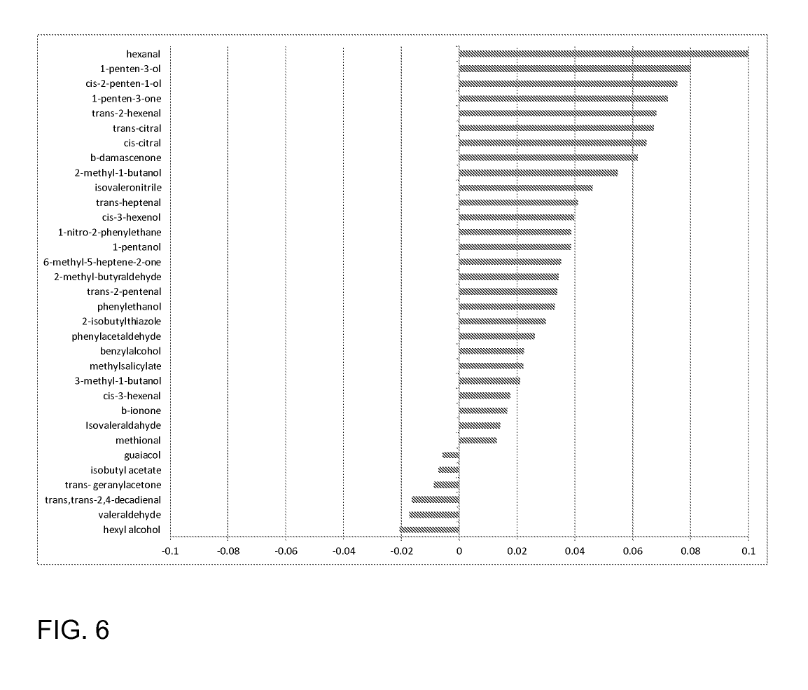

In studies evaluating aroma liking separately from taste, some volatiles contributed to consumer liking based on aroma (FIG. 6). Testing was conducted similarly as to the taste panels, except panelist evaluated the tomatoes based on smell prior to tasting the samples. Up to ten compounds appear to contribute to consumer aroma liking, including, but not limited to, hexanal, 1-penten-3-ol, cis-2-penten-1-ol, 1-penten-3-one, trans-2-hexenal, trans-citral, cis-citral, b-damascenone, 2-methyl-1-butanol, and isovaleronitrile. Such volatile compounds included 1-penten-3-one, trans-2-hexanal, and cis-2-penten-1-ol. Some of these compounds overlapped with volatile compounds with positive associations in the taste study, such as trans-2-hexanal, 1-penten-3-one, and cis-2-penten-1-ol. Thus, this study indicates that such compounds are also positively associated with overall liking.

Using linear regression modeling for the taste test data for each volatile (FIGS. 2A-2GG), it was possible to determine desirable levels for each volatile and to thus identify certain criteria associated with liking. In FIGS. 2AA-2GG, the volatiles that show an upward slant show a positive correlation to liking, a negative slant indicates a negative correlation to liking, and a generally horizontal line indicates the volatile is more or less neutral to taste/liking (FIG. 2 includes the linear regression modeling only for those volatile compounds that showed a fairly significant correlation to taste perception (e.g., liking)). This information was used to assemble a formula in which all volatiles are optimized, providing the chemical composition of a highly preferred tomato. The optimal formula was arbitrarily set for a liking value of 34, the highest value reported for any variety in the panels (Table 2). The formula for the value that panelists assigned for the “best tomato you ever tasted” was separately calculated to a score of 43. Such an “ideal” tomato is an average over the entire population. This information and regression analysis was used to determine a formula for levels of volatile compounds for the “most liked” tomato (liking score 34) and a hypothetical tomato with a liking score of 20, that would be considered a “well-liked” tomato (Table 3). Different individuals are likely to have different preferences. The design of the surveys permitted separation, for example, of tomato “lovers” from the larger population, and the ideal for this group is slightly different from the average values of the entire panel (data not shown).

There is a public perception that the term heirloom indicates a high quality tomato. The present results indicate that this is not always the case. Some heirloom varieties received liking scores well below the scores achieved by supermarket-purchased tomatoes (Tables 8A-8DD). These results do confirm the public perception of the average supermarket salad-type tomato. These tomatoes ranked 68 and 71 out of the 80 samples tested. Some of the top ranking tomatoes in the panel tests included, but are not necessarily limited to, Cherry Roma, Matina, Ailsa Craig, Red Calabash, Red Pear, Bloody Butcher, Maglia Rosa Cherry, Brandywine, Tommy Toe, Chadwick Cherry, Livingston's Stone, Super Sioux, St. Pierre, German Queen, Wisconsin 55, Micado Violettor, Livingston's Globe, and Gulf State Market (Tables 8A-8DD).

In conclusion, the present example provided a blueprint for how to define the ideal composition, or at least a goal composition, of a natural food crop. It is apparent that one cannot simply measure the content of each flavor-associated chemical and predict the impact of that chemical on flavor. The data reported here illustrate the complexity of flavor in a natural product and provide guidance for genetic improvement. The derived formula becomes the target for efforts to improve flavor quality through either molecular breeding or biotechnology. Further, through careful experimental design of sensory analysis, formulas can be customized according to preferences, demography or genetics. There have been extensive efforts to increase sugar content in modern tomato hybrids and there may not be much room for further improvement without negatively impacting yield. However, the molar levels of volatiles, by comparison, are much lower than the sugars and there is likely to be an opportunity to substantially increase synthesis of these compounds without a significant yield penalty. Of the volatiles that significantly impact flavor, most could be increased over the levels found in modern commercial cultivars, but a few negatively impact liking and could be reduced. The levels of each volatile in the formulas developed for the entire population fall within the range observed within the heirloom population. Based on this approach to improving tomato flavor, quantitative trait loci from donor lines of tomato can be identified, and, when introgressed, can deliver the desired increases and decreases in volatile content. Thus, it should be possible, with molecular-assisted breeding techniques to stack multiple alleles for the most important volatiles to significantly improve flavor quality.

### Methods

Plant material. Commercial tomato seeds were obtained from Seeds of Change (Santa Fe, N. Mex.), Totally Tomatoes (Randolph, Wis.) or Victory Seed Co. (Molalla, Oreg.). Most varieties selected were described as heirloom, open-pollinated varieties. A few modern hybrid varieties were also selected for comparison. Plants were grown in the field at the University of Florida North Florida Research and Education Center-Suwannee Valley in the spring or fall seasons or the greenhouse at Gainesville, Fla. S. lycopersicum var. cerasiforme seeds were obtained from the Tomato Genetics Resource Center, University of California, Davis, Calif. Supermarket tomatoes were obtained from a local supermarket in Gainesville, Fla.

Biochemical analysis. Volatile collection was performed as described in Tieman et al. (2006), which is incorporated by reference herein. Volatile compound identification was determined by GC-MS and co-elution with known standards (Sigma-Aldrich, St. Louis, Mo.). Sugars, acids and Brix were determined as described in Vogel et al. (2010), incorporated by reference herein.

Sensory analysis. Fully ripe fruit were harvested, and used for taste panels. Random fruit were used for biochemical analysis. A group of 170 tomato consumers (64 male, 106 female) were recruited to evaluate all the varieties. Panelists were between the ages of 18 and 78 with a median age of 22. Panelists self-classified themselves as 101 White/Caucasian, 14 Black/African-American, 32 Asian/Pacific and 25 Other. An average of 85 (range of 66-95) of these panelists evaluated between 4-6 varieties a session until all varieties were evaluated. All panelists went through a training session to familiarize them with the scales to be used and the procedures. Tomatoes were sliced into wedges (or in halves for grape/cherry types) and each panelist was given 2 pieces for evaluation. Panelists were instructed to chew and then swallow samples. They were instructed to take a bite of an unsalted cracker and a sip of water between samples. Samples were presented to the consumers in a randomized order. Panelists rated their overall liking as well as their liking for the texture on the hedonic gLMS (Bartoshuk, Fast & Snyder, 2005; Snyder et al, 2008). This scale assesses the liking for tomatoes in the context of all pleasure/displeasure experiences: 0=neutral, −100=strongest disliking of any kind experienced, and +100=strongest liking of any kind experienced. Panelists then rated their perceived intensities of overall tomato flavor, sweetness, sourness, saltiness and umami using the gLMS. This scale assesses taste and flavor sensations in the context of all sensory experience (Bartoshuk et al, 2002): 0=no sensation, 100=strongest sensation of any kind experienced. Both scales were devised to provide valid comparisons across subjects.

Statistical analysis. Statistical analysis was performed using Systat 13 software (Systat Software, Inc., Chicago, Ill.). Regression analysis was used to determine the biochemical composition of the ideal tomato with overall liking scores as the independent variable and the biochemical parameters as the dependent variables (FIGS. 2AA-2FF). Additional details regarding the statistical analysis are described below in Example 2.

LoxC transgenic tomatoes. A transformation vector containing the constitutive FMV 35S promoter (10, 21) a full-length antisense tomato 13-lipoxygenase LoxC (Chen et al., 2004 (19) open reading frame was introduced into S. lycopersicum var. M82 (31). Total RNA from fruit tissue was extracted with a Qiagen (Valencia, Calif.) Plant RNeasy kit followed by DNase treatment to remove contaminating DNA. RNA levels from 200 ng total RNA were measured using an Applied Biosystems (Carlsbad, Calif.) PowerSYBR Green RNA to C_{T} 1-step kit with forward primer 5′-GCAATGCATCATGTGTGCTA and reverse primer 5′-GTAAATGTCGAATTCCCTTCG. LoxC antisense tomato fruit RNA levels were 5% of control M82 fruit. Levels of the C6 volatiles hexyl alcohol, cis-3-hexenal, and cis-3-hexen-1-ol in LoxC antisense ripe fruit were less than 1% of control M82 fruit, whereas hexanal levels were less than 2% of control. Homozygous T2 plants were used for sensory analysis. Transgenic and M82 control fruits were harvested at the ripe stage. Seeds and locular material were removed and the remainder of the fruits used for taste panels. Random fruits were used for biochemical analysis. Seventy panelists (39% male, 61% female) were given two tomato samples (control v. transgenic) and asked to evaluate the texture, flavor and to describe how much they liked the sample using a 9-point hedonic scale. They were subsequently asked to identify the one that they preferred. No sample was preferred over the other in any of these evaluations (α=0.05). In a triangle test set-up, 59 panelists (42% male, 58% female) were given three samples (a triple combination of control and transgenic sample) and asked to identify the non-matching sample. The number of correct responses (29) was significant at α=0.01.

Transgenic sensory analysis. LoxC antisense fruit and control M82 fruit were grown in the field at the University of Florida North Florida Research and Education Center-Suwannee Valley in the fall of 2010. Transgenic and control fruit were harvested at the ripe stage. Seeds and locular material were removed and the remainder of the fruit was used for taste panels. Random fruit were used for biochemical analysis. Seventy panelists (39% male, 61% female) were given 2 tomato samples (control v. transgenic) and asked to evaluate the texture, flavor and to describe how much they liked the sample using the 9-point hedonic scale. Following this, they were also asked to identify the one they preferred. No sample was preferred over the other in any of these evaluations (α=0.05). In a triangle test set-up, 59 panelists (42% male, 58% female) were given 3 samples (a triple combination of control and transgenic sample) and were asked to identify the non-matching sample. The number of correct responses (29) was significant at α=0.01.

### Example 2

### The Relationship Between Chemistry and Preferences

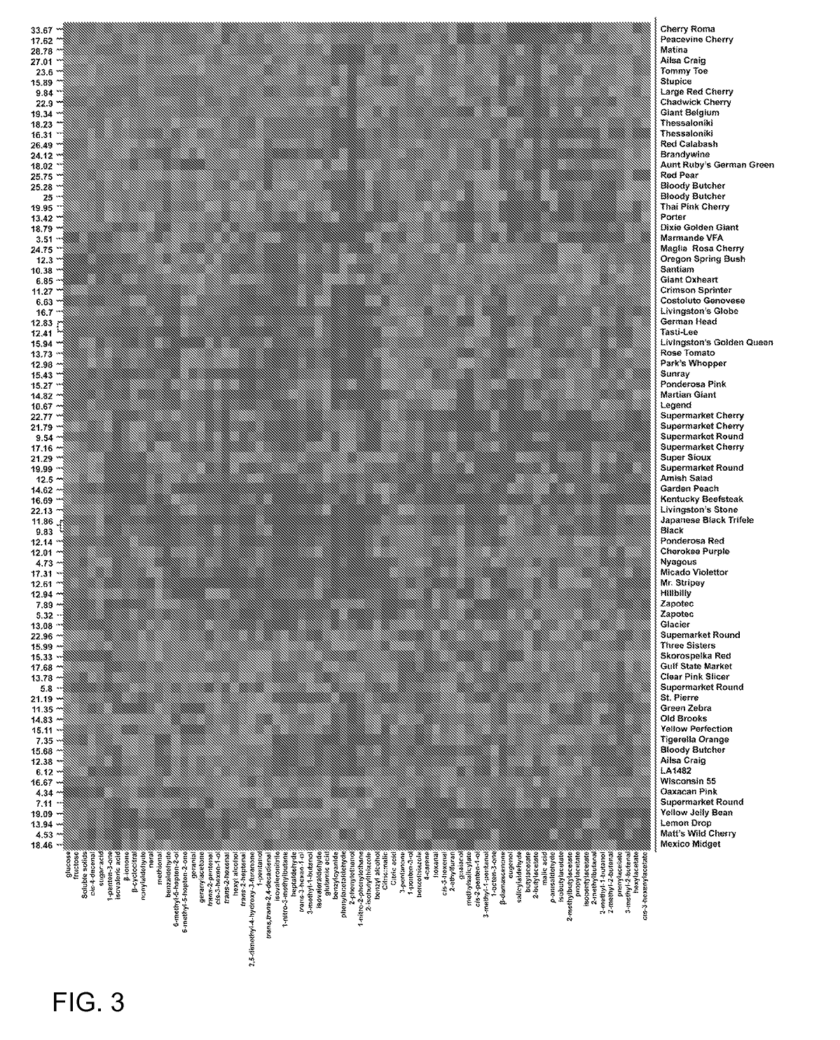

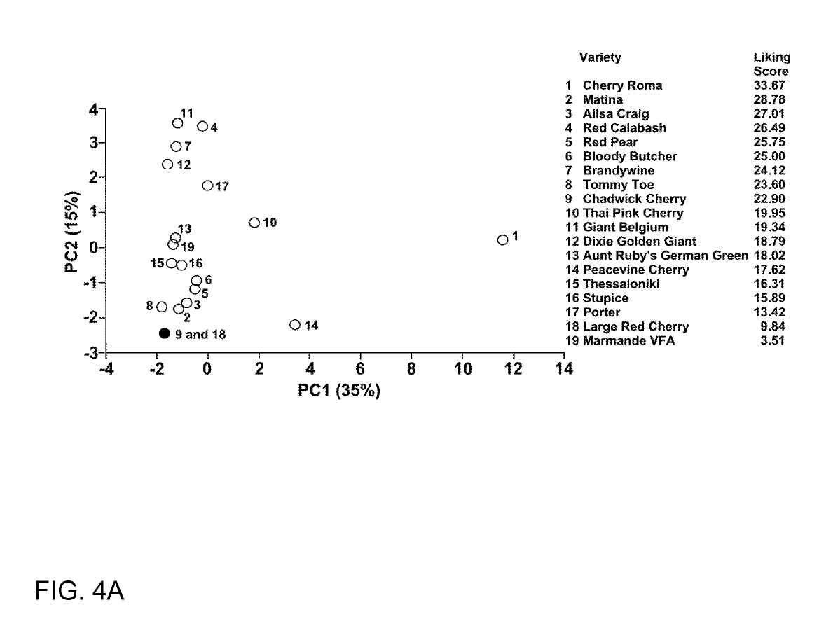

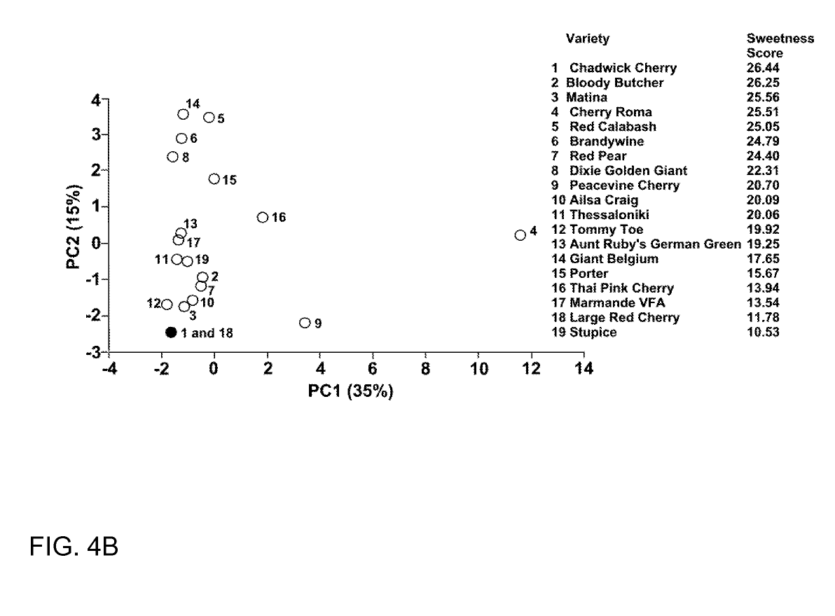

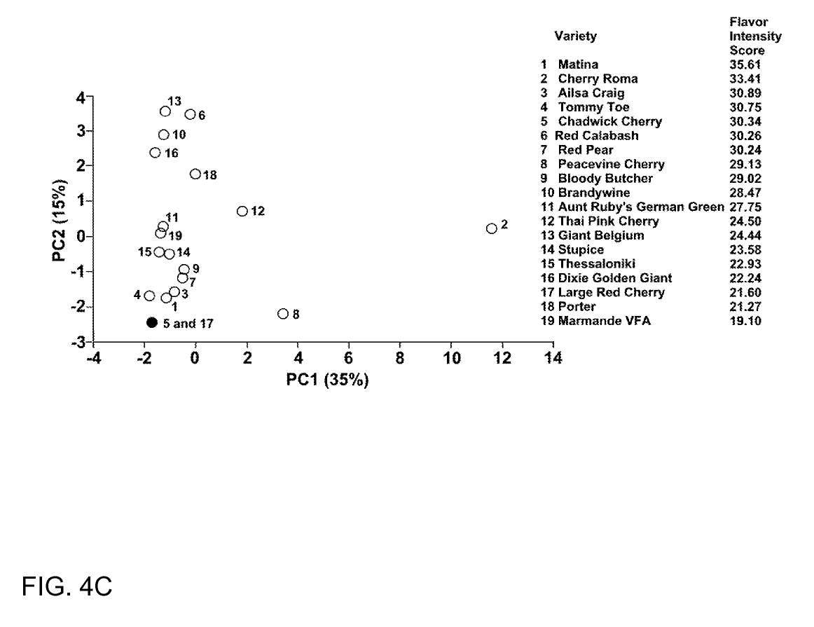

Since a close genetic relationship among highly liked or disliked varieties could potentially bias any effort to associate chemical composition with consumer preferences, to address this concern, the genetic relationships of 19 varieties that were grown and subjected to consumer evaluations in a single season were examined. A set of 27 biomarkers that are polymorphic within cultivated tomato were used to genotype each variety (FIG. 4). Based on these data no obvious genetic subgroups that could explain liking, sweetness or tomato flavor intensity were found. There is no obvious genetic clustering of good vs. bad taste when varieties were sorted by chemical composition (FIG. 3). These latter data also indicate the chemical complexity of liking as there is no simple pattern of chemical content that separates high or low consumer liking scores.

Due to the large number of chemicals potentially influencing liking, a multivariate analysis of the data was performed. The attributes were initially partitioned into six groups based upon chemical properties and biosynthetic pathways: sugars, branched chain amino acids, fatty acids, carotenoids, phenolics, and acids. Compounds for which biosynthetic pathways are not established were assigned to one of the six classes based upon their correlations with other classified compounds (24). Groups of structurally related chemicals with known metabolic links were examined for compounds within each module that were highly colinear and compounds that were upstream in relevant metabolic pathways were preferentially selected. The selection process reduced the set to 27 compounds (Table 6). Flavor intensity was associated with 12 different compounds, seven of which were independently significant after accounting for fructose: 2-butylacetate, cis-3-hexen-1-ol, citric acid, 3-methyl-1-butenol, 2-methylbutanal, 1-octen-3-one and trans,trans-2,4-decadienal. Sweetness was associated with 12 compounds, eight of which overlap with those important for flavor and three of which are independent predictors of sweetness after accounting for fructose: geranial, 2-methylbutanal and 3-methyl-1-butanol.

Interactions between taste (e.g., sweetness) and retronasal olfaction are of considerable interest in the chemical senses (26). The present example provides evidence for these interactions in a natural food product: the tomato and influence on overall liking. Although sweetness of tomatoes is widely thought to result from sugars, volatiles proved to be important contributors to sweetness. Volatiles are perceived in two ways. They can be sniffed through the nostrils (orthonasal olfaction) or when foods containing volatiles and chewed and swallowed, volatiles are forced up behind the palate into the nasal cavity from the back (retronasal olfaction). Orthonasal olfaction is commonly called “smell;” retronasal olfaction contributes to “flavor.” Retronasal olfaction and taste interact in the brain. Commonly paired taste and retronasal olfactory sensations can become associated such that either sensation can induce the other centrally. Instances of volatile-induced tastes of sweet, sour, bitter and salty have been observed (27). Multiple regression with sweetness as the dependent variable showed that the perception of tomato flavor (retronasal olfaction) made a significant contribution to sweetness after accounting for fructose (p<0.0001). Similarly, tomato flavor made a significant contribution to sourness that was independent of citric acid (p<0.001). Interestingly, one of the volatiles that contributed to this sourness, 2-methylbutanal, was negatively correlated with sweetness. This result provides some insight into how different tastes induced centrally by volatiles may interact.

The contributions, or lack thereof, for certain volatiles were somewhat unexpected. Prior lists of important tomato flavor volatiles were compiled based largely on odor unit values (25). The data from example 1 indicate that some of these volatiles with high odor unit values, such as β-damascenone and phenylacetaldehyde, are not associated with tomato flavor liking or intensity although they have historically been considered to be important contributors to flavor (25). These results indicate that these volatiles should not be considered high priority targets for genetic manipulations.

Due to interactions between taste and retronasal olfaction, there were correlations between certain volatiles and sugars that contribute to the liking of tomato fruits. Notably, the apocarotenoid geranial was positively correlated with sweetness. Tomato mutants specifically deficient in carotenoid biosynthesis are deficient in apocarotenoid volatiles, including geranial, 6-methyl-5-hepten-2-one and β-ionone, but unaltered in sugars, acids and non-apocarotenoid volatiles. They are perceived as less sweet by consumers, validating the contribution of geranial to sweetness (23). Consistent with a model in which liking is also a function of sweetness and flavor, apocarotenoid-deficient fruits are also significantly less liked by consumers. In a complementary experiment, Baldwin et al., (28) have shown that adding sugars or acids can alter the perception of tomato aroma volatiles.

The positive association of sweet perception and liking with volatiles such as geranial suggests that consumer liking of tomatoes can be enhanced by increasing the concentrations of certain volatiles such as geranial in the fruit.

### Conclusions

The present example exploited the natural chemical variation within tomato to determine the chemical interactions that drive consumer liking. These data illustrate the challenge of understanding flavor, and consumer preferences in particular, in a natural product. Starting with a large set of chemically distinct volatiles, efforts can now be focused at genetic improvement on a smaller set than previously thought possible. Despite the large number of QTL that impact flavor chemicals (2, 3, 29), it should be possible with molecular-assisted breeding techniques to exploit the natural variation present within the heirloom population, combining desirable alleles of multiple genes to significantly improve flavor quality. While consumer liking was averaged across the entire population for the present example, the data permit separation of preferences by age, sex, body mass and genetics (30). The collected data permit defining the parameters of a better tasting tomato to the average consumer in the United States, with the possibility of optimization for specific groups. Taken together, the results provide new insights into flavor and liking and illustrate the flaws in a traditional approach based on odor units. The presence of a molecule, even at a relatively high level, does not mean that it significantly contributes to either flavor or liking. Models based on concentration and odor thresholds of individual volatiles cannot account for synergistic and antagonistic interactions that occur in complex foods such as a tomato fruit.

Materials & Methods

Molecular marker analysis. A standard protocol was used to isolate genomic DNA from young leaves of each variety. From a total of 36 markers, the following 27 were polymorphic within the set of 19 tomato varieties with liking scores: CosOH51, LEOH1.1, LEOH16.2, LEOH18, LEOH36, LEOH19, LEOH70, Rx3-L1, SP, SSR20, SSR43, SSR47, SSR63, SSR111, SSR115, SSR128, SSR134, SSR318, SSR306, TOM144, SL10126-1067i, SL10184-480i, SL10615-428i, SL20210-883i, OVATE, FAS, and LC (31, 32). The CAPS markers were scored on 2-4% agarose gels whereas the Simple Sequence Repeat (SSR) and Indels were scored on the LI-COR IR2 4200 (LI-COR Biosciences, Lincoln, Nebr., USA). There was 1.6% missing marker data. The missing data were imputed by replacing the missing value with the most frequent allele for that marker in the entire dataset. Principal Component Analysis (PCA) was performed using Minitab 15.1.0.0 Software. To combine SSR with Single Nucleotide Polymorphism (SNP) data, the allele sizes were used. To avoid bias due to allele size difference, the PCA was done with the covariance matrix.

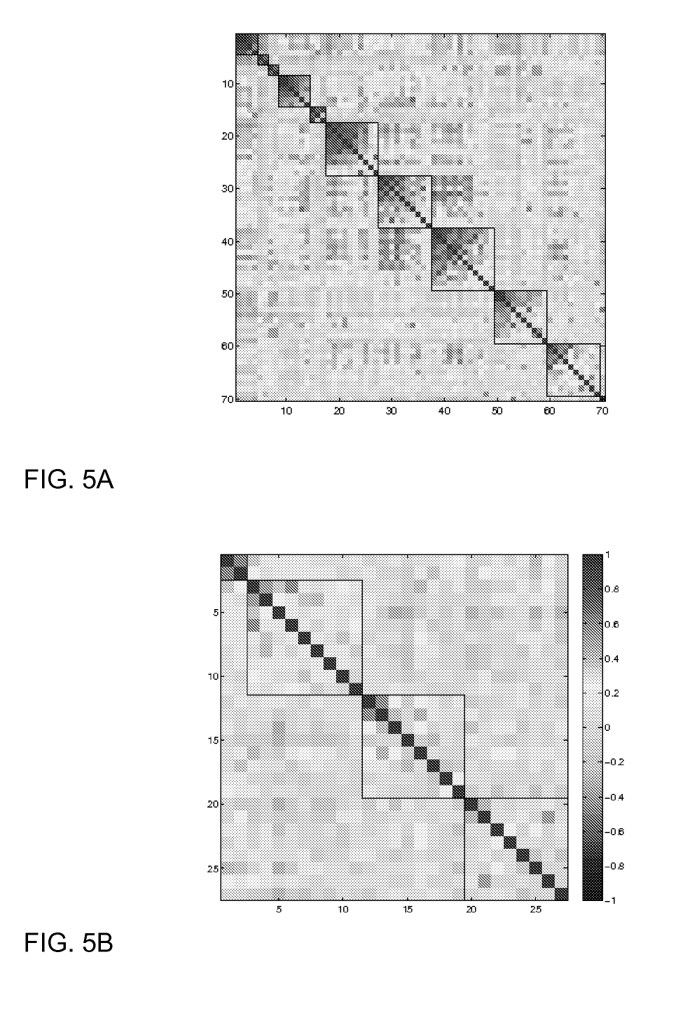

Statistical analysis. The 68 chemical compounds measured in this experiment were divided into six groups based upon biochemical properties: sugars, branched chain amino acids, lipids, carotenoids, phenolics, and acids. A small number of compounds for which biosynthetic pathways are not established were assigned to one of the six classes based upon their correlations with other classified compounds. All pairwise correlations among the set of 68 compounds were calculated. Correlation coefficients were sorted using Modulated Modularity Clustering (MMC) (24) as a visual aid for identifying compounds that are closely related in this sample (FIG. 5A; Table 6). Biochemical groups were examined for compounds within the group that were highly correlated and compounds that were upstream in the relevant metabolic pathways were preferentially selected. The selection process resulted in 27 compounds (FIG. 5A, Table 6) that were representative of each of the 6 biochemical groups, and limited the amount of correlation between compounds. The set of 27 was examined using MMC and the result confirmed that the pairwise correlation had been reduced (FIG. 5B). An exploratory factor analysis did not reveal obvious structure among the remaining compounds. For example the lipids did not all load together on a single factor. The relationship between overall liking, sweetness and flavor intensity was modeled using a multivariate linear regression. The model Yᵢⱼ=μ+Sᵢⱼ+Fᵢⱼ+εᵢⱼ was fit where Y is the overall liking score for variety i in panel j; S is the sweetness, F is the flavor intensity measured as described above and ε is the error. Sweetness and flavor intensity contributed to overall liking, and flavor intensity remained an independent predictor (p=0.0393) of overall liking even after accounting for sweetness. To determine whether volatiles contributed to either of these components of liking, we modeled sweetness and flavor in a linear model.

Benzothiazole, butylacetate, cis-3-hexen-1-ol, citric acid, fructose, geranial, methional, 3-methyl-1-butenol, 2-methylbutanal, 1-octen-3-one, phenylacetaldehyde and trans,trans-2,4,decadienal were associated with flavor intensity in univariate models. 2-Butylacetate, cis-3-hexen-1-ol, citric acid, 3-methyl-1-butenol, 2-methylbutanal, 1-octen-3-one and trans, trans-2,4-decadienal were significant after accounting for fructose. Butylacetate, 4-carene, cis-3-hexen-1-ol, eugenol, fructose, geranial guaiacol, heptaldehyde, methional, 3-methyl-1-butenol, 2-methylbutanal, and phenylacetaldehyde all showed evidence for association with sweetness in univariate models and geranial, 3-methyl-1-butenol, 2-methylbutanal were significant after accounting for fructose. The final fitted multivariate model included fructose (p<0.0001), geranial (p=0.038) and 2-methylbutanal (p=0.015). For flavor intensity benzothiazole, butylacetate, cis-3-hexen-1-ol, citric acid, fructose, geranial, methional, 3-methyl-1-butenol, 2-methylbutanal, 1-octen-3-one, phenylacetaldehyde and trans,trans-2,4,decadienal were associated with flavor intensity in univariate models. 2-Butylacetate, cis-3-hexen-1-ol, citric acid, 3-methyl-1-butenol, 2-methylbutanal, 1-octen-3-one and trans,trans-2,4-decadienal were significant after accounting for fructose. Either 1-octen-3-one or 3-methyl-1-butenol could be included in the final model. The final model included fructose (p<0.0001), 1-octen-3-one (p=0.0026), cis-3-hexen-1-ol (p=0.0092), 2-methylbutanal (p=0.0191), citric acid (p=0.0003), trans,trans-2,4-decadienal (p<0.0001) 2-butlyacetate (p=0.038) and butylacetate (p=0.0143). All analyses were performed in SAS v 9.2.

### Example 3

The present example describes new hybrid tomato varieties that have been produced by crossing an elite, commercial tomato with different heirloom tomato varieties selected from some of the higher scoring tomatoes from the tasting panels described in Example 1. The following crosses were produced:

Flora-dade by German Queen

Flora-dade by Matina

Flora-dade by Wisconsin 55

German Queen by Flora-dade

Matina by Flora-dade

Wisconsin 55 by Flora-dade

The elite tomato parent for all crosses was the Flora-dade, and the other tomato is the heirloom variety, selected from German Queen, Matina, and Wisconsin 55. The female parent is listed first in each cross.

The hybrid tomatoes were grown in the greenhouse or field. The initial F1 tomatoes from the above crosses were harvested in November 2010.

The fruit from the hybrid tomatoes from each cross, as well as fruits from each parent cultivar were tested in a Tasting panel conducted as described in Example 1. Initial results of the tasting panels are summarized in Table 7, below. The results indicate the fruit of the F1 hybrids have improved taste (as indicated by higher liking scores) over fruit of the elite tomato variety parent and that the hybrids have improved texture than the parent heirloom varieties. The F1 hybrid plants were also observed to perform better in the field than the parent heirloom varieties.

The following references are incorporated by reference herein in pertinent part:
1. Saliba-Colombani V, Causse M, Langlois D, Philouze J, Buret M. 2001. Genetic analysis of organoleptic quality in fresh market tomato. 1. Mapping QTLs for physical and chemical traits. Theoretical and Applied Genetics 102: 259-272.
2. Tieman D, Zeigler M, Schmelz E, Taylor M, Bliss P, Kirst M, Klee H. 2006. Identification of loci affecting flavor volatile emissions in tomato fruits. J. Exp. Bot. 57: 887-896.
3. Mathieu S, Dal Cin V, Fei Z, Li H, Bliss P, Taylor M G, Klee H J, Tieman DM. 2009. Flavor compounds in tomato fruits: identification of loci and potential pathways affecting volatile composition. J. Exp. Bot. 60: 325-337.
4. Baldwin E A, Scott J W, Shewmaker C K, Schuch W. 2000. Flavor trivia and tomato aroma: biochemistry and possible mechanisms for control of important aroma components. Hortscience 35: 1013-1022.
5. Goff S A, Klee H J. 2006. Plant volatile compounds: sensory cues for health and nutritional value. Science 311: 815-819.
6. Klee H. 2010. Tansley Review: Improving the taste and flavor of fresh fruits: Genomics, biochemistry, and biotechnology. New Phytol. 187: 44-56
7. Buttery, R G, Teranishi R, Flath R A, Ling L C. 1987. Fresh tomato volatiles: composition and sensory studies. pp. 213-222. In: Teranishi R, Buttery R, Shahidi R (eds.). Flavor chemistry: Trends and developments. Amer. Chem. Soc. Symposium Series, Washington, D.C.
8. Malnic B, Hirono J, Sato T, Buck L. 1999. Combinatorial Receptor Codes for Odors. Cell 96:713-723
9. Baldwin E A, Goodner K, Plotto A. 2008. Interaction of volatiles, sugars, and acids on perception of tomato aroma and flavor descriptors. J. Food Science 73: S294-S307
10. Rick CM. 1995. Lycopersicon esculentum. pp 452-457. In: Evolution of crop plants, Smartt J and Simmonds N W eds. Longman Scientific and Technical, Harlow UK.
11. Jiménez-Gómez J M, Maloof J N. 2009. Sequence diversity in three tomato species: SNPs, markers, and molecular evolution. BMC Plant Biology 9:85
12. Bartoshuk L M, Duffy V B, Fast K, Green B G, Prutkin J M, Snyder D J. 2002. Labeled scales (e.g., category, Likert, VAS) and invalid across-group comparisons. What we have learned from genetic variation in taste. Food Quality and Preference 14: 125-138.
13. Bartoshuk L M, Fast K, Snyder D J. 2005. Differences in our sensory worlds: Invalid comparisons with labeled scales. Current Directions in Psychological Science 14: 122-125.
14. Centeno D, et al. 2011. Malate plays a crucial role in starch metabolism, ripening, and soluble solid content of tomato fruit and affects postharvest Softening. Plant Cell 23:162-184
15. Chen G, Hackett R, Walker D, Taylor A, Lin Z, Grierson D. 2004. Identification of a specific isoform of tomato lipoxygenase (TomloxC) involved in the generation of fatty acid-derived flavor compounds. Plant Physiology 136: 2641-2651.
16. Acknowledgements. This work was supported in part by a grant from the National Science Foundation to HJK (IOS-0923312), the Institute for Food and Agricultural Sciences and the Vice President for Research. We especially wish to thank the summer interns from Fort Valley State University for their help. Special thanks to Howard Shapiro and Seeds of Change for their donation of heirloom tomatoes.
17. Bartoshuk, L. M., V. B. Duffy, K. Fast, B. G. Green, J. M. Prutkin & D. J. Snyder (2002). “Labeled scales (e.g., category, Likert, VAS) and invalid across-group comparisons. What we have learned from genetic variation in taste.” Food Quality and Preference 14: 125-138.
18. Bartoshuk, L. M., K. Fast & D. J. Snyder (2005). “Differences in our sensory worlds: Invalid comparisons with labeled scales.” Current Directions in Psychological Science 14: 122-125.
19. Chen G, Hackett R, Walker D, Taylor A, Lin Z, Grierson D (2004) Identification of a specific isoform of tomato lipoxygenase (TomloxC) involved in the generation of fatty acid-derived flavor compounds. Plant Physiol. 136:2641-2651.
20. McCormick S, Neidermeyer J, Fry J, Barnason A, Horsch R, Fraley R (1986) Leaf disc transformation of cultivated tomato (L. esculentum) using Agrobacterium tumefaciens. Plant Cell Rep. 5:81-84.
21. Snyder, D. J., L. A. Puentes, C. A., Sims & L. M. Bartoshuk (2008). “Building a better intensity scale: Which labels are essential?” Chemical Senses 33: S142.
22. Tieman D, Zeigler M, Schmelz E, Taylor M, Bliss P, Kirst M, Klee H. 2006. Identification of loci affecting flavor volatile emissions in tomato fruits. J. Exp. Bot. 57: 887-896.
23. Vogel J T, Tieman D M, Sims C A, Odabasi A Z, Clark D G, Klee H J (2010) Carotenoid content impacts flavor acceptability in tomato (Solanum lycopersicum). J. Sci. Food Agric. 90:2233-2240.
24. Stone, E. A., Ayroles, J. M. (2009). Modulated Modularity Clustering as an Exploratory Tool for Functional Genomic Inference. PLoS Genet 5(5): e1000479. doi:10.1371/journal.pgen.1000479.
25. Buttery, R. G., Teranishi, R., Flath, R. A., Ling, L. C. (1987). Fresh tomato volatiles: composition and sensory studies. In: Teranishi R., Buttery R., Shahidi R., eds. Flavor chemistry: Trends and developments. (Amer. Chem. Soc. Symposium Series, Washington, D.C.). pp. 213-222.
26. Noble, A. C. (1996). Taste-aroma interactions. Trends Food Sci. Technol. 7, 439-444.
27. Salles, C. (2006) Odour-taste interactions in flavor perception. In: Voilley, A., Etiévant, P. eds. Flavour in Food. (Woodhead Publishing Ltd., Cambridge, UK), pp 345-368.
28. Baldwin, E. A., Goodner, K., Plotto, A. (2008). Interaction of volatiles, sugars, and acids on perception of tomato aroma and flavor descriptors. J. Food Sci. 73, S294-S307.
29. Causse, M., Saliba-Colombani, V., Buret, M., Lesschaeve, I., Issanchou, S. (2001). Genetic analysis of organoleptic quality in fresh market tomato. 2. Mapping QTLs for sensory attributes. Theor. Appl. Genet. 102, 273-283.
30. Bartoshuk, L. M., Blandon, A., Bliss, P. L., Clark, D. G., Colquhoun, T. A., Klee, H. J., Moskowitz, H. K., Sims, C. A. Snyder, D. K., and Tieman, D. M. (2011). Better tomatoes through psychophysics. Chem. Senses, 31, A118.
31. Rodriguez, G. R., Muños, S., Anderson, C., Sim, S.-C., Michel, A., Causse, M., McSpadden Gardener, B. B., Francis, D., van der Knaap, E. (2011). Distribution of SUN, OVATE, LC, and FAS in the Tomato Germplasm and the Relationship to Fruit Shape Diversity. Plant Physiol. 156, 275-285.
32. Rodriguez, G. R., Muños, S., Anderson, C., Sim, S.-C., Michel, A., Causse, M., McSpadden Gardener, B. B., Francis, D., van der Knaap, E. (2011). Distribution of SUN, OVATE, LC, and FAS in the Tomato Germplasm and the Relationship to Fruit Shape Diversity. Plant Physiol. 156, 275-285.

TABLE 1
List of the flavor chemicals correlated with consumer liking and
perceived tomato flavor intensity. Chemicals are sorted by p-value.

| | Overall | Flavor |
|---|---|---|
| | liking | intensity |
| Compound | p-value | p-value |
| glucose | 0 | 0 |
| fructose | 0 | 0 |
| soluble solids | 0 | 0 |
| 1-penten-3-one | 0 | 0 |
| trans-2-pentenal | 0 | 0 |
| trans-2-heptenal | 0 | 0 |
| trans-3-hexen-1-ol | 0 | 0 |
| 6-methyl-5-hepten-2-ol | 0 | 0 |
| nonyl aldehyde | 0 | 0.001 |
| isovaleronitrile | 0 | 0.003 |
| sugar:acid ratio | 0 | 0.018 |
| cis-4-decenal | 0 | 0.044 |
| 3-methyl-1-butanol | 0.001 | 0 |
| 2,5-dimethyl-4-hydroxy-3(2H)-furanone | 0.001 | 0.001 |
| 1-pentanol | 0.001 | 0.001 |
| methional | 0.001 | 0.004 |
| benzyl cyanide | 0.001 | 0.004 |
| isovaleraldehyde | 0.001 | 0.051 |
| 3-pentanone | 0.001 | 0.221 |
| 2-isobutylthiazole | 0.002 | 0 |
| benzaldehyde | 0.002 | 0 |
| isovaleric acid | 0.002 | 0.006 |
| 1-nitro-3-methylbutane | 0.002 | 0.041 |
| β-ionone | 0.003 | 0.033 |
| β-cyclocitral | 0.003 | 0.072 |
| 6-methyl-5-hepten-2-one | 0.003 | 0.095 |
| geranial | 0.004 | 0.032 |
| phenylacetaldehyde | 0.005 | 0.008 |
| eugenol | 0.006(−) | 0.077 |
| geranylacetone | 0.008 | 0.008 |
| 2-phenyl ethanol | 0.009 | 0.006 |
| neral | 0.012 | 0.001 |
| salicylaldehyde | 0.013(−) | 0.002(−) |
| isobutyl acetate | 0.016(−) | 0.38 |
| butyl acetate | 0.019(−) | 0.002(−) |
| cis-3-hexen-1-ol | 0.022 | 0.007 |
| 1-nitro-2-phenylethane | 0.024 | 0.005 |
| 1-penten-3-ol | 0.025 | 0.491 |
| 2-methylbutyl acetate | 0.027(−) | 0.608 |
| heptaldehyde | 0.03 | 0.279 |
| trans,trans-2,4-decadienal | 0.034 | 0.005 |
| malic acid | 0.037(−) | 0.358 |
| 2-methylbuteraldehyde | 0.04(−) | 0.001(−) |
| 4-carene | 0.042 | 0.194 |
| hexyl alcohol | 0.048 | 0.031 |
| guaiacol | 0.06 | 0.245 |
| propyl acetate | 0.065 | 0.475 |
| hexanal | 0.069 | 0.052 |
| cis-2-penten-1-ol | 0.087 | 0.999 |
| glutamic acid | 0.088 | 0.124 |
| 2-butyl acetate | 0.099 | 0.835 |
| 1-octen-3-one | 0.126 | 0.004 |
| cis-3-hexenal | 0.22 | 0.229 |
| methylsalicylate | 0.236 | 0.034 |
| trans-2-hexenal | 0.252 | 0.01 |
| β-damascenone | 0.253 | 0.578 |
| 2-methyl-1-butanol | 0.255 | 0.004 |
| 2-methyl-2-butenal | 0.269 | 0.542 |
| prenyl acetate | 0.299 | 0.39 |
| hexyl acetate | 0.376 | 0.816 |
| citric:malic ratio | 0.402 | 0.042 |
| 3-methyl-1-pentanol | 0.469 | 0.768 |
| 2-ethylfuran | 0.611 | 0.196 |
| isopentyl acetate | 0.625 | 0.057 |
| benzothiazole | 0.643 | 0.059 |
| cis-3-hexenyl acetate | 0.665 | 0.64 |
| benzyl alcohol | 0.761 | 0.064 |
| citric acid | 0.863 | 0.001 |
| 3-methyl-2-butenal | 0.941 | 0.773 |
| p-anisaldehyde | 0.953 | 0.934 |

(−) indicates negative correlation.

TABLE 2
Formula of the most liked tomato. Ideal formula was determined by regression analysis
setting liking value at 34 (the highest rating given to a tomato variety actually tasted
in all panels) or 43 (the average liking value of the best tomato ever tasted by the panelists
(the “idealized” tomato)). The range observed in the tested population for each chemical
as well as the fold difference from high to low are shown. Volatile levels are ng
gFW⁻¹ h⁻¹, sugars and acids are mg gFW⁻¹.

| | | Formula | | | |
|---|---|---|---|---|---|
| | Formula | idealized | | | |
| | liking | tomato | Highest | Lowest | Fold |
| | value of 34 | 43 | concentration | concentration | difference |
| glucose | 25.3 | 30.8 | 29.7 | 5.1 | 5.8 |
| fructose | 27.9 | 33.1 | 35.8 | 8.6 | 4.1 |
| Soluble solids | 7.1 | 8.1 | 8.5 | 3.4 | 2.5 |
| 1-penten-3-one | 2.8 | 3.7 | 7.5 | 0.23 | 32 |
| isovaleronitrile | 25.1 | 32.5 | 58.2 | 0.7 | 80 |
| trans-2-pentenal | 1.8 | 2.2 | 5.4 | 0.1 | 41 |
| trans-2-heptenal | 0.77 | 0.99 | 2.76 | 0.03 | 81 |
| trans-3-hexen-1-ol | 1.8 | 2.2 | 3.4 | 0.2 | 20 |
| 6-methyl-5-hepten- | 0.28 | 0.35 | 0.68 | 0.01 | 66 |
| 2-ol | | | | | |
| nonyl aldehyde | 0.46 | 0.57 | 1.13 | 0.06 | 19 |
| cis-4-decenal | 1.8 | 2.3 | 5.6 | 0.2 | 23 |
| sugar:acid ratio | 13.1 | 15.7 | 15.7 | 2.0 | 7.7 |
| isovaleraldehyde | 21.2 | 26.0 | 59.2 | 1.1 | 54 |
| 3-methyl-1-butanol | 62.3 | 77.8 | 155.3 | 2.5 | 62 |
| methional | 0.24 | 0.29 | 0.55 | 0.02 | 24 |
| 2,5-dimethyl-4- | 0.74 | 0.98 | 3.61 | 0.01 | 246 |
| hydroxy-3(2H)- | | | | | |
| furanone | | | | | |
| 3-pentanone | 8.5 | 9.8 | 12.6 | 1.5 | 8.6 |
| 1-pentanol | 5.3 | 6.4 | 16.9 | 1.1 | 16 |
| benzyl cyanide | 0.63 | 0.86 | 3.32 | 0.01 | 395 |
| isovaleric acid | 0.14 | 0.18 | 0.43 | 0.003 | 123 |
| 2-isobutylthiazole | 9.1 | 11.3 | 24.8 | 0.5 | 47 |
| 1-nitro-3- | 33.2 | 42.6 | 104.0 | 0.6 | 188 |
| methylbutane | | | | | |
| benzaldehyde | 4.9 | 6.0 | 13.4 | 0.3 | 39 |
| 6-methyl-5-hepten- | 5.8 | 6.8 | 9.2 | 0.1 | 80 |
| 2-one | | | | | |
| β-ionone | 0.10 | 0.12 | 0.46 | 0.01 | 74 |
| β-cyclocitral | 0.15 | 0.19 | 0.59 | 0.01 | 50 |
| geranial | 0.21 | 0.25 | 0.50 | 0.005 | 101 |
| phenylacetaldehyde | 0.52 | 0.68 | 2.77 | 0.01 | 266 |
| eugenol | 0.004 | 0 | 3.40 | 0.001 | 3628 |
| geranylacetone | 2.4 | 3.0 | 9.6 | 0.02 | 418 |
| 2-phenylethanol | 1.4 | 1.9 | 7.9 | 0.003 | 2634 |
| neral | 0.20 | 0.22 | 0.40 | 0.04 | 10 |
| salicylaldehyde | 0.29 | 0.06 | 2.39 | 0.02 | 106 |
| isobutyl acetate | 0.33 | 0 | 7.09 | 0.13 | 54 |
| butyl acetate | 0.08 | 0.02 | 0.68 | 0 | 635 |
| cis-3-hexen-1-ol | 53.1 | 61.7 | 197.0 | 6.0 | 33 |
| 1-nitro-2- | 1.2 | 1.5 | 4.2 | 0.01 | 362 |
| phenylethane | | | | | |
| 1-penten-3-ol | 5.7 | 6.4 | 13.0 | 1.5 | 8.9 |
| 2-methylbutyl | 0.35 | 0 | 7.14 | 0.04 | 162 |
| acetate | | | | | |
| heptaldehyde | 6.4 | 8.0 | 29.1 | 0.3 | 95 |
| trans,trans-2,4- | 0.02 | 0.03 | 0.13 | 0.001 | 139 |
| decadienal | | | | | |
| malic acid | 0.39 | 0.29 | 1.64 | 0.18 | 8.9 |
| 2- | 3.1 | 2.5 | 8.5 | 1.1 | 7.4 |
| methylbuteraldehyde | | | | | |
| 4-carene | 0.04 | 0.05 | 0.16 | 0.003 | 51 |
| hexyl alcohol | 31.9 | 38.7 | 176.5 | 1.5 | 116 |
| guaiacol | 1.2 | 1.5 | 4.9 | 0.01 | 331 |
| propyl acetate | 0.18 | 0.12 | 1.04 | 0.03 | 36 |
| hexanal | 131.8 | 148.6 | 306.8 | 13.5 | 23 |
| cis-2-penten-1-ol | 1.5 | 1.6 | 3.2 | 0.4 | 9.1 |
| glutamic acid | 2.4 | 2.7 | 9.0 | 0.6 | 16 |
| 2-butylacetate | 0.05 | 0 | 1.1 | 0.003 | 431 |
| 1-octen-3-one | 0.08 | 0.10 | 0.41 | 0.01 | 34 |
| cis-3-hexenal | 89.1 | 98.4 | 198.3 | 6.9 | 29 |
| methylsalicylate | 0.68 | 0.79 | 2.47 | 0.003 | 855 |
| trans-2-hexenal | 6.0 | 6.9 | 30.3 | 0.3 | 105 |
| β-damascenone | 0.0022 | 0 | 0.104 | 0.001 | 116 |
| 2-methyl-1-butanol | 17.6 | 19.5 | 43.0 | 2.1 | 20 |
| 2-methyl-2-butenal | 6.6 | 5.7 | 22.6 | 1.5 | 16 |
| prenyl acetate | 0.011 | 0.005 | 0.19 | 0.001 | 243 |
| hexyl acetate | 0.33 | 0.28 | 2.03 | 0.015 | 137 |
| citric:malic | 9.9 | 10.7 | 29.3 | 1.6 | 19 |
| 3-methyl-1-pentanol | 0.78 | 0.85 | 2.79 | 0.02 | 154 |
| 2-ethylfuran | 0.11 | 0.10 | 0.32 | 0.01 | 33 |
| isopentyl acetate | 0.31 | 0.33 | 1.32 | 0.00 | 2275 |
| benzothiazole | 0.07 | 0.07 | 0.14 | 0.01 | 16 |
| cis-3-hexenyl | 1.7 | 1.7 | 4.4 | 0.5 | 8.3 |
| acetate | | | | | |
| benzyl alcohol | 0.38 | 0.36 | 2.55 | 0.03 | 96 |
| citric acid | 3.7 | 3.8 | 6.7 | 1.5 | 4.5 |
| 3-methyl-2-butenal | 0.36 | 0.36 | 2.25 | 0.07 | 30 |
| p-anisaldehyde | 0.008 | 0.008 | 0.07 | 0.001 | 68 |

TABLE 3
Recipe of tomato with liking value 20. Ideal recipe was determined by regression analysis
setting liking value at 33.67 (the highest rating given to a tomato variety in all panels) or 20
(the liking value of a tomato that would be considered well-liked). The range observed in the
tested population for each chemical as well as the fold difference from high to low are
shown. Volatile levels are ng gFW⁻¹ h⁻¹, sugars and acids are mg gFW⁻¹.

| | | Recipe | | | |
|---|---|---|---|---|---|
| | Recipe | idealized | | | |
| | liking | tomato | Highest | Lowest | Fold |
| | value of 34 | 20 | concentration | concentration | difference |
| glucose | 25.3 | 17.1 | 29.7 | 5.1 | 5.8 |
| fructose | 27.9 | 20.1 | 35.8 | 8.6 | 4.1 |
| TSS | 7.1 | 5.6 | 8.5 | 3.4 | 2.5 |
| 1-penten-3-one | 2.8 | 1.6 | 7.5 | 0.23 | 32 |
| isovaleronitrile | 25.1 | 14.0 | 58.2 | 0.7 | 80 |
| trans-2-pentenal | 1.8 | 1.1 | 5.4 | 0.1 | 41 |
| trans-2-heptenal | 0.77 | 0.44 | 2.76 | 0.03 | 81 |
| trans-3-hexen-1-ol | 1.8 | 1.2 | 3.4 | 0.2 | 20 |
| 6-methyl-5-hepten- | 0.28 | 0.17 | 0.68 | 0.01 | 66 |
| 2-ol | | | | | |
| nonyl aldehyde | 0.46 | 0.30 | 1.13 | 0.06 | 19 |
| cis-4-decenal | 1.8 | 1.2 | 5.6 | 0.2 | 23 |
| sugar:acid ratio | 13.1 | 9.2 | 15.7 | 2.0 | 7.7 |
| isovaleraldehyde | 21.2 | 14.1 | 59.2 | 1.1 | 54 |
| 3-methyl-1-butanol | 62.3 | 39.1 | 155.3 | 2.5 | 62 |
| methional | 0.24 | 0.16 | 0.55 | 0.02 | 24 |
| 2,5-dimethyl-4- | 0.74 | 0.39 | 3.61 | 0.01 | 246 |
| hydroxy-3(2H)- | | | | | |
| furanone | | | | | |
| 3-pentanone | 8.5 | 6.6 | 12.6 | 1.5 | 8.6 |
| 1-pentanol | 5.3 | 3.8 | 16.9 | 1.1 | 16 |
| benzyl cyanide | 0.63 | 0.29 | 3.32 | 0.01 | 395 |
| isovaleric acid | 0.14 | 0.08 | 0.43 | 0.003 | 123 |
| 2-isobutylthiazole | 9.1 | 5.9 | 24.8 | 0.5 | 47 |
| 1-nitro-3- | 33.2 | 19.1 | 104.0 | 0.6 | 188 |
| methylbutane | | | | | |
| benzaldehyde | 4.9 | 3.1 | 13.4 | 0.3 | 39 |
| 6-methyl-5-hepten- | 5.8 | 4.2 | 9.2 | 0.1 | 80 |
| 2-one | | | | | |
| β-ionone | 0.10 | 0.06 | 0.46 | 0.01 | 74 |
| β-cyclocitral | 0.15 | 0.10 | 0.59 | 0.01 | 50 |
| geranial | 0.21 | 0.15 | 0.50 | 0.005 | 101 |
| phenylacetaldehyde | 0.52 | 0.29 | 2.77 | 0.01 | 266 |
| eugenol | 0.004 | 0.48 | 3.40 | 0.001 | 3628 |
| geranylacetone | 2.4 | 1.61 | 9.6 | 0.02 | 418 |
| 2-phenylethanol | 1.4 | 0.7 | 7.9 | 0.003 | 2634 |
| neral | 0.20 | 0.16 | 0.40 | 0.04 | 10 |
| salicylaldehyde | 0.29 | 0.63 | 2.39 | 0.02 | 106 |
| isobutyl acetate | 0.33 | 0.95 | 7.09 | 0.13 | 54 |
| butyl acetate | 0.08 | 0.17 | 0.68 | 0 | 635 |
| cis-3-hexen-1-ol | 53.1 | 40.2 | 197.0 | 6.0 | 33 |
| 1-nitro-2- | 1.2 | 0.73 | 4.2 | 0.01 | 362 |
| phenylethane | | | | | |
| 1-penten-3-ol | 5.7 | 4.7 | 13.0 | 1.5 | 8.9 |
| 2-methylbutyl | 0.35 | 1.0 | 7.14 | 0.04 | 162 |
| acetate | | | | | |
| heptaldehyde | 6.4 | 3.9 | 29.1 | 0.3 | 95 |
| trans,trans-2,4- | 0.02 | 0.01 | 0.13 | 0.001 | 139 |
| decadienal | | | | | |
| malic acid | 0.39 | 0.53 | 1.64 | 0.18 | 8.9 |
| 2- | 3.1 | 3.9 | 8.5 | 1.1 | 7.4 |
| methylbuteraldehyde | | | | | |
| 4-carene | 0.04 | 0.03 | 0.16 | 0.003 | 51 |
| hexyl alcohol | 31.9 | 21.7 | 176.5 | 1.5 | 116 |
| guaiacol | 1.2 | 0.9 | 4.9 | 0.01 | 331 |
| propyl acetate | 0.18 | 0.28 | 1.04 | 0.03 | 36 |
| hexanal | 131.8 | 106.7 | 306.8 | 13.5 | 23 |
| cis-2-penten-1-ol | 1.5 | 1.2 | 3.2 | 0.4 | 9.1 |
| glutamic acid | 2.4 | 1.9 | 9.0 | 0.6 | 16 |
| 2-butylacetate | 0.05 | 0.14 | 1.1 | 0.003 | 431 |
| 1-octen-3-one | 0.08 | 0.06 | 0.41 | 0.01 | 34 |
| cis-3-hexenal | 89.1 | 75.2 | 198.3 | 6.9 | 29 |
| methylsalicylate | 0.68 | 0.52 | 2.47 | 0.003 | 855 |
| trans-2-hexenal | 6.0 | 4.6 | 30.3 | 0.3 | 105 |
| β-damascenone | 0.0022 | 0.006 | 0.104 | 0.001 | 116 |
| 2-methyl-1-butanol | 17.6 | 14.7 | 43.0 | 2.1 | 20 |
| 2-methyl-2-butenal | 6.6 | 8.1 | 22.6 | 1.5 | 16 |
| prenyl acetate | 0.011 | 0.018 | 0.19 | 0.001 | 243 |
| hexyl acetate | 0.33 | 0.41 | 2.03 | 0.015 | 137 |
| citric:malic | 9.9 | 8.8 | 29.3 | 1.6 | 19 |
| 3-methyl-1-pentanol | 0.78 | 0.68 | 2.79 | 0.02 | 154 |
| 2-ethylfuran | 0.11 | 0.11 | 0.32 | 0.01 | 33 |
| isopentyl acetate | 0.31 | 0.28 | 1.32 | 0.00 | 2275 |
| benzothiazole | 0.07 | 0.07 | 0.14 | 0.01 | 16 |
| cis-3-hexenyl | 1.7 | 1.6 | 4.4 | 0.5 | 8.3 |
| acetate | | | | | |
| benzyl alcohol | 0.38 | 0.41 | 2.55 | 0.03 | 96 |
| citric acid | 3.7 | 3.7 | 6.7 | 1.5 | 4.5 |
| 3-methyl-2-butenal | 0.36 | 0.35 | 2.25 | 0.07 | 30 |
| p-anisaldehyde | 0.008 | 0.008 | 0.07 | 0.001 | 68 |

TABLE 4
Observed variation in flavor volatiles within S. lycopersicum heirloom
varieties. Volatile emissions were measured as ng/fresh weight/hr.

| | | | Fold | |
|---|---|---|---|---|
| | High | Low | difference | Median |
| 1-penten-3-one | 9.37 | 0.17 | 55 | 1.18 |
| isovaleronitrile | 68.45 | 0.58 | 117 | 7.63 |
| trans-2-pentenal | 5.16 | 0.31 | 17 | 1.23 |
| trans-2-heptenal | 2.71 | 0.09 | 30 | 0.42 |
| isovaleraldehyde | 51.08 | 1.55 | 33 | 8.59 |
| 3-methyl-1-butanol | 184.46 | 3.20 | 58 | 27.26 |
| methional | 1.616 | 0.012 | 137 | 0.07 |
| isovaleric acid | 0.953 | 0.004 | 262 | 0.09 |
| 2-isobutylthiazole | 63.61 | 0.37 | 174 | 8.34 |
| 6-methyl-5-hepten- | 20.07 | 0.17 | 120 | 3.38 |
| 2-one | | | | |
| β-ionone | 0.396 | 0.008 | 47 | 0.05 |
| phenylacetaldehyde | 1.90 | 0.00 | 654 | 0.24 |
| geranylacetone | 28.96 | 0.03 | 1095 | 1.22 |
| 2-phenylethanol | 5.269 | 0.002 | 3142 | 0.05 |
| isobutyl acetate | 11.93 | 0.14 | 85 | 1.67 |
| cis-3-hexen-1-ol | 124.15 | 10.00 | 12 | 40.00 |
| 1-nitro-2- | 2.59 | 0.02 | 149 | 0.25 |
| phenylethane | | | | |
| trans,trans-2,4- | 0.30 | 0.00 | 211 | 0.02 |
| decadienal | | | | |
| 2-methylbutanal | 14.66 | 1.14 | 13 | 3.47 |
| hexyl alcohol | 84.03 | 0.99 | 85 | 13.86 |
| guaiacol | 8.09 | 0.03 | 290 | 0.77 |
| hexanal | 381.05 | 15.55 | 25 | 88.65 |
| 1-octen-3-one | 0.312 | 0.017 | 18 | 0.07 |
| cis-3-hexenal | 399.66 | 8.29 | 48 | 71.09 |
| methylsalicylate | 14.16 | 0.00 | 3354 | 0.40 |
| trans-2-hexenal | 48.01 | 0.39 | 123 | 3.54 |
| β-damascenone | 0.1733 | 0.0020 | 86 | 0.01 |
| 2-methyl-1-butanol | 115.69 | 1.93 | 60 | 15.08 |

TABLE 5
C6 volatile emission in fruit of control (M82) and LoxC antisense plants. Volatile
emissions (ng gFW⁻¹ h⁻¹) from ripe control (M82) and transgenic (LoxCAS) fruits
were measured as described in supplementary materials and methods.

| | cis-3- | | cis-3-hexen-1- | | |
|---|---|---|---|---|---|
| | hexenal | hexanal | ol | hexyl alcohol | hexyl acetate |
| M82 | 139 ± 55 | 202 ± 43 | 59.0 ± 14.6 | 38.7 ± 11.2 | 2.61 ± 0.85 |
| LoxCAS-0966 | 0.6 ± 0.1 | 1.9 ± 0.3 | 0.07 ± 0.01 | 0.08 ± 0.01 | 0.014 ± 0.002 |

TABLE 6
List of the 68 chemical measurements used in flavor analysis.
Correlation coefficients were sorted into 11 modules using MMC
(Stone et al., 2009). The 27 individual compounds used in the
multivariate models are shown in bold.

| | | | Average | |
|---|---|---|---|---|
| Chemical | Module | EntryIndex | Degree | Degree |
| glucose | 1 | 4 | 0.79652 | 0.87055 |
| fructose | 1 | 5 | 0.79652 | 0.85714 |
| Solublesolids | 1 | 1 | 0.79652 | 0.78411 |
| Sugar:acidratio | 1 | 8 | 0.79652 | 0.67427 |
| salicylaldehyde | 2 | 61 | 0.70129 | 0.70129 |
| eugenol | 2 | 70 | 0.70129 | 0.70129 |
| cis-3-hexenylacetate | 3 | 58 | 0.69388 | 0.69388 |
| hexylacetate | 3 | 59 | 0.69388 | 0.69388 |
| phenylacetaldehyde | 4 | 28 | 0.6015 | 0.70973 |
| 2-phenylethanol | 4 | 31 | 0.6015 | 0.67879 |
| benzylcyanide | 4 | 63 | 0.6015 | 0.67478 |
| 1-nitro-2-phenylethane | 4 | 33 | 0.6015 | 0.5788 |
| benzaldehyde | 4 | 55 | 0.6015 | 0.49608 |
| nonylaldehyde | 4 | 62 | 0.6015 | 0.47085 |
| Citric:malicratio | 5 | 7 | 0.5326 | 0.68885 |
| Citric acid | 5 | 2 | 0.5326 | 0.45591 |
| Malic acid | 5 | 3 | 0.5326 | 0.45304 |
| isovaleronitrile | 6 | 12 | 0.48128 | 0.61834 |
| trans-3-hexen-1-ol | 6 | 49 | 0.48128 | 0.60896 |
| 1-nitro-3-methylbutane | 6 | 53 | 0.48128 | 0.58995 |
| 3-methyl-1-butanol | 6 | 13 | 0.48128 | 0.56904 |
| isovaleraldehyde | 6 | 9 | 0.48128 | 0.54462 |
| heptaldehyde | 6 | 52 | 0.48128 | 0.50809 |
| 2-isobutylthiazole | 6 | 27 | 0.48128 | 0.45552 |
| Isovalericacid | 6 | 19 | 0.48128 | 0.43208 |
| Glutamic acid | 6 | 6 | 0.48128 | 0.27246 |
| benzylalcohol | 6 | 60 | 0.48128 | 0.21373 |
| geranial | 7 | 69 | 0.43585 | 0.57172 |
| β-cyclocitral | 7 | 65 | 0.43585 | 0.54336 |
| 6-methyl-5-hepten-2-one | 7 | 26 | 0.43585 | 0.53271 |
| β-ionone | 7 | 37 | 0.43585 | 0.51036 |
| 6-methyl-5-hepten-2-ol | 7 | 57 | 0.43585 | 0.49241 |
| neral | 7 | 66 | 0.43585 | 0.4174 |
| methional | 7 | 23 | 0.43585 | 0.35934 |
| hexanal | 7 | 18 | 0.43585 | 0.35458 |
| geranylacetone | 7 | 36 | 0.43585 | 0.35061 |
| 4-carene | 7 | 56 | 0.43585 | 0.22603 |
| trans-2-heptenal | 8 | 24 | 0.43028 | 0.60483 |
| 2,5-dimethyl-4-hydroxy-3- | 8 | 29 | 0.43028 | 0.57533 |
| furanone | | | | |
| 1-pentanol | 8 | 44 | 0.43028 | 0.5473 |
| trans-2-pentenal | 8 | 15 | 0.43028 | 0.50386 |
| cis-3-hexen-1-ol | 8 | 21 | 0.43028 | 0.49881 |
| 1-penten-3-one | 8 | 11 | 0.43028 | 0.47961 |
| hexylalcohol | 8 | 22 | 0.43028 | 0.44961 |
| cis-4-decenal | 8 | 64 | 0.43028 | 0.38099 |
| trans,trans-2,4-decadienal | 8 | 34 | 0.43028 | 0.35884 |
| trans-2-hexenal | 8 | 20 | 0.43028 | 0.35151 |
| 1-octen-3-one | 8 | 25 | 0.43028 | 0.29723 |
| p-anisaldehyde | 8 | 68 | 0.43028 | 0.11544 |
| isobutylacetate | 9 | 16 | 0.36278 | 0.53598 |
| 2-methylbutylacetate | 9 | 51 | 0.36278 | 0.5311 |
| propylacetate | 9 | 41 | 0.36278 | 0.46039 |
| 2-methyl-2-butenal | 9 | 42 | 0.36278 | 0.37115 |
| butylacetate | 9 | 47 | 0.36278 | 0.3455 |
| isopentylacetate | 9 | 50 | 0.36278 | 0.32623 |
| 2-methyl-1-butanol | 9 | 14 | 0.36278 | 0.32153 |
| prenylacetate | 9 | 54 | 0.36278 | 0.29625 |
| 3-methyl-2-butenal | 9 | 46 | 0.36278 | 0.29397 |
| 2-butylacetate | 9 | 43 | 0.36278 | 0.14571 |
| cis-2-penten-1-ol | 10 | 45 | 0.2872 | 0.46808 |
| 1-penten-3-ol | 10 | 38 | 0.2872 | 0.41541 |
| cis-3-hexenal | 10 | 17 | 0.2872 | 0.35648 |
| 3-pentanone | 10 | 39 | 0.2872 | 0.32567 |
| 3-methyl-1-pentanol | 10 | 48 | 0.2872 | 0.27314 |
| 2-ethylfuran | 10 | 40 | 0.2872 | 0.26516 |
| guaiacol | 10 | 30 | 0.2872 | 0.23619 |
| benzothiazole | 10 | 67 | 0.2872 | 0.2024 |
| methylsalicylate | 10 | 32 | 0.2872 | 0.19102 |
| β-damascenone | 10 | 35 | 0.2872 | 0.13845 |
| 2-methylbutanal | 11 | 10 | 0 | 0 |

TABLE 7
hybrid tomato taste panel results

| Variety | Overall Liking | Texture | Sweetness |
|---|---|---|---|
| Flora-Dade x Wisconsin 55 | 18.2 | 15.8 | 14.6 |
| Wisconsin 55 | 16.1 | 9.9 | 15.0 |
| Wisconsin 55 x Flora-Dade | 11.4 | 5.5 | 14.4 |
| Flora-Dade | 7.6 | 14.1 | 9.8 |
| Flora-Dade x Wisconsin55 | 13.8 | 13.4 | 13.2 |
| Wisconsin 55 | 10.9 | 10.5 | 11.6 |
| Wisconsin 55 x Flora-Dade | 9.9 | 13.8 | 10.1 |
| Flora-Dade | 6.9 | 11.0 | 8.9 |
| Matina x Flora-Dade | 14.5 | 16.9 | 12.1 |
| Flora-Dade x Matina | 14.4 | 17.2 | 11.8 |
| Matina | 9.4 | 11.4 | 9.5 |
| Flora-Dade | 7.6 | 14.1 | 9.8 |
| German Queen x Flora- | 16.1 | 17.9 | 13.0 |
| Dade | | | |
| Flora-Dade x German | 14.8 | 18.5 | 12.4 |
| Queen | | | |
| German Queen | 10.3 | 9.2 | 11.9 |
| Flora-Dade | 4.2 | 9.3 | 7.3 |

Tables 8A-8DD: Taste panel and biochemical data for 66 tomato varieties. Taste panels were performed over three seasons and fruit were either grown in the field or a greenhouse. Tomatoes purchased from a local supermarket were also tasted and analyzed.

TABLE 8A

| | | | | | | | | | Tomato |
|---|---|---|---|---|---|---|---|---|---|
| | | Overall | Texture | | | | | | Flavor |
| | | Liking | liking | Sweetness | Sourness | Salty | Bitter | Umami | Intenstity |
| | panel | panel | panel | panel | panel | panel | panel | panel | panel |
| Variety | date | score | score | score | score | score | score | score | score |
| Cherry Roma | Feb. 24, 2010 | 33.67 | 34.06 | 25.51 | 18.20 | 12.94 | 9.93 | 12.86 | 33.41 |
| Ailsa Craig | Feb. 24, 2010 | 27.01 | 20.99 | 20.09 | 17.10 | 11.11 | 11.43 | 13.52 | 30.89 |
| Red Pear | Feb. 24, 2010 | 25.75 | 23.25 | 24.40 | 12.90 | 10.40 | 6.85 | 10.19 | 30.24 |
| Tommy Toe | Feb. 24, 2010 | 23.60 | 17.23 | 19.92 | 13.26 | 11.47 | 8.32 | 11.43 | 30.75 |
| Supermarket Cherry | Feb. 24, 2010 | 21.79 | 25.14 | 14.18 | 18.06 | 12.14 | 10.48 | 13.50 | 28.07 |
| Porter | Feb. 24, 2010 | 13.42 | 9.86 | 15.67 | 9.01 | 7.94 | 8.34 | 11.22 | 21.27 |
| Matina | Mar. 2, 2010 | 28.78 | 27.47 | 25.56 | 16.53 | 12.56 | 11.22 | 16.67 | 35.61 |
| Bloody Butcher | Mar. 2, 2010 | 25.28 | 17.62 | 25.91 | 10.90 | 10.04 | 7.87 | 11.98 | 30.20 |
| Chadwick Cherry | Mar. 2, 2010 | 22.90 | 14.81 | 26.44 | 11.34 | 10.46 | 7.16 | 12.01 | 30.34 |
| Thai Pink Cherry | Mar. 2, 2010 | 19.95 | 19.03 | 13.94 | 15.50 | 10.00 | 9.97 | 11.53 | 24.50 |
| Peacevine Cherry | Mar. 2, 2010 | 17.62 | 18.93 | 20.70 | 14.37 | 11.18 | 8.63 | 12.64 | 29.13 |
| Supermarket Cherry | Mar. 2, 2010 | 17.16 | 18.86 | 11.46 | 23.51 | 13.77 | 13.20 | 11.24 | 30.36 |
| Stupice | Mar. 2, 2010 | 15.89 | 19.05 | 10.53 | 15.27 | 11.24 | 14.56 | 12.24 | 23.58 |
| Large Red Cherry | Mar. 2, 2010 | 9.84 | 9.15 | 11.78 | 14.80 | 13.82 | 12.20 | 9.20 | 21.60 |
| Bloody Butcher | Mar. 18, 2010 | 25.00 | 19.63 | 26.25 | 9.78 | 9.74 | 4.58 | 9.07 | 29.02 |
| Giant Belgium | Mar. 18, 2010 | 19.34 | 8.04 | 17.65 | 9.83 | 8.99 | 6.96 | 12.18 | 24.44 |
| Thessaloniki | Mar. 18, 2010 | 18.23 | 11.71 | 17.82 | 10.45 | 8.64 | 7.26 | 9.37 | 22.82 |
| Supermarket Round | Mar. 18, 2010 | 5.80 | 8.74 | 7.51 | 14.54 | 10.03 | 11.47 | 10.31 | 19.84 |
| Marmande VFA | Mar. 18, 2010 | 3.51 | −2.54 | 13.54 | 8.89 | 8.24 | 8.24 | 9.62 | 19.10 |
| Red Calabash | Apr. 1, 2010 | 26.49 | 21.19 | 25.05 | 16.73 | 11.53 | 7.68 | 8.96 | 30.26 |
| Brandywine | Apr. 1, 2010 | 24.12 | 23.81 | 24.79 | 13.95 | 10.49 | 5.46 | 11.01 | 28.47 |
| Dixie Golden Giant | Apr. 1, 2010 | 18.79 | 9.05 | 22.31 | 10.04 | 7.95 | 5.40 | 9.75 | 22.24 |
| Aunt Ruby's German Green | Apr. 1, 2010 | 18.02 | 18.72 | 19.25 | 12.34 | 10.47 | 6.95 | 11.28 | 27.75 |
| Thessaloniki | Apr. 1, 2010 | 16.31 | 13.40 | 20.06 | 9.62 | 8.40 | 7.91 | 9.28 | 22.93 |
| Supermarket Round | Apr. 1, 2010 | 9.54 | 18.44 | 7.87 | 19.12 | 10.75 | 12.82 | 11.31 | 25.60 |

TABLE 8B

| | | | | | | | | | Tomato |
|---|---|---|---|---|---|---|---|---|---|
| | | Overall | Texture | | | | | | Flavor |
| | | Liking | liking | Sweetness | Sourness | Salty | Bitter | Umami | Intenstity |
| | | panel | panel | panel | panel | panel | panel | panel | panel |
| Variety | panel date | score | score | score | score | score | score | score | score |
| Supermarket Round | Jun. 11, 2010 | 22.96 | 23.88 | 15.24 | 11.88 | 10.48 | 9.14 | 12.73 | 26.75 |
| Three Sisters | Jun. 11, 2010 | 15.99 | 18.10 | 18.63 | 14.79 | 11.04 | 9.98 | 14.31 | 31.29 |
| Skorospelka Red | Jun. 11, 2010 | 15.33 | 11.46 | 12.59 | 26.21 | 13.15 | 13.08 | 11.00 | 32.14 |
| Yellow Perfection | Jun. 11, 2010 | 15.11 | 17.66 | 17.00 | 18.96 | 10.70 | 9.20 | 10.39 | 28.68 |
| Clear Pink Slicer | Jun. 11, 2010 | 13.78 | 8.05 | 19.65 | 8.29 | 7.86 | 5.78 | 11.30 | 26.98 |
| Tigerella Orange | Jun. 11, 2010 | 7.35 | 10.93 | 11.44 | 16.29 | 11.51 | 11.96 | 10.41 | 22.45 |
| Gulf State Market | Jun. 17, 2010 | 17.68 | 14.10 | 16.48 | 17.45 | 11.78 | 9.22 | 14.18 | 32.89 |
| Bloody Butcher | Jun. 17, 2010 | 15.68 | 11.10 | 18.71 | 12.96 | 9.21 | 7.72 | 12.39 | 28.11 |
| Garden Peach | Jun. 17, 2010 | 14.62 | 22.29 | 10.62 | 19.16 | 8.70 | 8.96 | 10.60 | 26.32 |
| Amish Salad | Jun. 17, 2010 | 12.50 | 9.61 | 14.78 | 11.57 | 7.24 | 6.18 | 10.37 | 23.38 |
| Ailsa Craig | Jun. 17, 2010 | 12.38 | 12.79 | 15.00 | 12.83 | 8.54 | 9.18 | 11.72 | 24.65 |
| Supermarket Round | Jun. 17, 2010 | 7.11 | 14.37 | 8.80 | 13.06 | 8.65 | 9.09 | 10.94 | 21.05 |
| Super Sioux | Jun. 30, 2010 | 21.29 | 14.27 | 15.79 | 13.12 | 7.53 | 6.64 | 10.65 | 28.40 |
| St. Pierre | Jun. 30, 2010 | 21.19 | 14.86 | 20.51 | 11.00 | 8.19 | 5.42 | 11.15 | 28.00 |
| Supermarket Round | Jun. 30, 2010 | 19.99 | 21.65 | 12.94 | 8.09 | 7.27 | 7.42 | 8.86 | 21.91 |
| Kentucky Beefsteak | Jun. 30, 2010 | 16.69 | 18.68 | 14.54 | 11.63 | 7.32 | 7.04 | 10.12 | 22.68 |
| Green Zebra | Jun. 30, 2010 | 11.35 | 18.65 | 8.29 | 29.62 | 10.95 | 10.88 | 9.51 | 32.60 |
| Zapotec | Jun. 30, 2010 | 7.89 | 13.91 | 10.10 | 7.94 | 8.51 | 7.27 | 9.63 | 18.79 |
| Supermarket Cherry | Jul. 8, 2010 | 22.77 | 22.49 | 19.08 | 7.68 | 7.71 | 4.36 | 8.75 | 25.43 |
| Yellow Jelly Bean | Jul. 8, 2010 | 19.09 | 17.49 | 19.64 | 9.12 | 7.87 | 6.05 | 10.42 | 26.47 |
| Mexico Midget | Jul. 8, 2010 | 18.46 | 17.62 | 13.20 | 16.13 | 10.29 | 9.57 | 9.92 | 27.88 |
| Lemon Drop | Jul. 8, 2010 | 13.94 | 17.50 | 16.22 | 14.28 | 7.83 | 5.66 | 9.33 | 26.63 |
| LA1482 | Jul. 8, 2010 | 6.12 | 12.63 | 9.38 | 19.26 | 10.08 | 12.00 | 8.43 | 26.92 |
| Matt's Wild Cherry | Jul. 8, 2010 | 4.53 | 6.43 | 13.29 | 11.83 | 7.62 | 11.05 | 9.08 | 22.57 |

TABLE 8C

| | | Overall | Texture | | | | | | Flavor |
|---|---|---|---|---|---|---|---|---|---|
| | | Liking | liking | Sweetness | Sourness | Salty | Bitter | Umami | Intenstity |
| Variety | panel date | score | score | score | score | score | score | score | score |
| Maglia Rosa Cherry | Nov. 3, 2010 | 24.75 | 22.35 | 16.50 | 16.43 | 9.25 | | 9.57 | 26.88 |
| Wisconsin 55 | Nov. 3, 2010 | 16.67 | 6.58 | 11.85 | 12.88 | 8.68 | | 10.40 | 22.38 |
| Glacier | Nov. 3, 2010 | 13.08 | 18.69 | 11.49 | 15.00 | 8.74 | | 10.48 | 22.76 |
| Oregon Spring Bush | Nov. 3, 2010 | 12.30 | 10.50 | 9.13 | 12.38 | 7.55 | | 9.20 | 17.92 |
| Santiam | Nov. 3, 2010 | 10.38 | 13.01 | 8.52 | 16.20 | 10.37 | | 10.52 | 23.23 |
| Giant Oxheart | Nov. 3, 2010 | 6.85 | 8.20 | 8.05 | 13.48 | 8.92 | | 9.94 | 17.73 |
| Micado Violettor | Nov. 12, 2010 | 17.31 | 19.09 | 10.91 | 10.81 | 7.50 | 4.49 | 8.20 | 18.91 |
| Livingston's Globe | Nov. 12, 2010 | 16.70 | 12.43 | 14.35 | 10.05 | 7.34 | 4.09 | 8.23 | 19.91 |
| Old Brooks | Nov. 12, 2010 | 14.83 | 13.20 | 14.48 | 9.13 | 7.31 | 4.33 | 11.06 | 19.93 |
| Park's Whopper | Nov. 12, 2010 | 12.98 | 16.23 | 10.83 | 12.06 | 7.25 | 5.43 | 9.36 | 20.03 |
| Hillbilly | Nov. 12, 2010 | 12.94 | 14.40 | 12.80 | 7.89 | 5.99 | 3.29 | 8.01 | 17.46 |
| German Head | Nov. 12, 2010 | 12.83 | 13.08 | 9.44 | 11.29 | 8.36 | 6.54 | 8.50 | 17.69 |
| Livingston's Stone | Nov. 17, 2010 | 22.13 | 17.69 | 14.10 | 12.00 | 8.15 | 6.58 | 9.91 | 23.52 |
| Ponderosa Pink | Nov. 17, 2010 | 15.27 | 11.46 | 9.73 | 11.40 | 7.64 | 6.06 | 8.91 | 19.07 |
| Cherokee Purple | Nov. 17, 2010 | 12.01 | 10.11 | 11.80 | 11.12 | 8.40 | 7.32 | 8.96 | 20.27 |
| Black | Nov. 17, 2010 | 9.83 | 10.42 | 10.90 | 9.85 | 7.88 | 5.14 | 9.49 | 17.20 |
| Nyagous | Nov. 17, 2010 | 4.73 | 9.23 | 8.31 | 7.11 | 6.15 | 5.11 | 8.12 | 14.62 |
| Oaxacan Pink | Nov. 17, 2010 | 4.34 | 6.31 | 6.49 | 20.46 | 10.77 | 10.00 | 8.81 | 24.68 |
| Livingston's Golden Queen | Nov. 23, 2010 | 15.94 | 17.52 | 10.80 | 15.64 | 9.27 | 6.55 | 8.62 | 23.42 |
| Ponderosa Red | Nov. 23, 2010 | 12.14 | 16.27 | 9.03 | 11.59 | 7.88 | 4.33 | 7.79 | 17.59 |
| Japanese Black Trifele | Nov. 23, 2010 | 11.86 | 16.08 | 12.85 | 7.52 | 6.82 | 4.20 | 7.76 | 16.33 |
| Crimson Sprinter | Nov. 23, 2010 | 11.27 | 13.36 | 7.79 | 17.48 | 9.21 | 5.83 | 9.39 | 22.29 |
| Legend | Nov. 23, 2010 | 10.67 | 12.92 | 9.17 | 10.53 | 7.45 | 5.45 | 8.67 | 18.59 |
| Zapotec | Nov. 23, 2010 | 5.32 | 13.00 | 6.85 | 6.89 | 6.47 | 5.61 | 8.77 | 14.70 |
| Sunray | Dec. 1, 2010 | 15.43 | 18.15 | 8.85 | 11.98 | 7.71 | 5.07 | 8.29 | 19.38 |
| Martian Giant | Dec. 1, 2010 | 14.82 | 13.76 | 9.10 | 9.18 | 6.44 | 4.93 | 7.89 | 17.67 |
| Rose Tomato | Dec. 1, 2010 | 13.73 | 14.83 | 8.10 | 12.89 | 8.22 | 6.63 | 8.15 | 19.79 |
| Mr. Stripey | Dec. 1, 2010 | 12.61 | 17.80 | 9.30 | 8.12 | 6.10 | 6.72 | 7.91 | 15.24 |
| Tasti-Lee | Dec. 1, 2010 | 12.41 | 15.49 | 6.72 | 13.93 | 8.77 | 7.17 | 9.18 | 19.51 |
| Costoluto Genovese | Dec. 1, 2010 | 6.63 | 11.98 | 6.38 | 14.90 | 8.71 | 7.57 | 9.15 | 19.68 |

TABLE 8D

| | | Soluble | | | | citric | malic | | glutamatic |
|---|---|---|---|---|---|---|---|---|---|
| | panel | solids | glucose | fructose | sugar:acid | acid | acid | citric:malic | acid |
| Variety | date | Brix | mg/gfw | mg/gfw | ratio | mg/gfw | mg/gfw | ratio | mg/gfw |
| Cherry Roma | Feb. 24, 2010 | 8.10 | 26.84 | 27.80 | 11.13 | 4.70 | 0.21 | 22.76 | 3.53 |
| Ailsa Craig | Feb. 24, 2010 | 5.20 | 14.51 | 16.69 | 8.56 | 3.00 | 0.64 | 4.68 | 1.40 |
| Red Pear | Feb. 24, 2010 | 6.55 | 21.01 | 23.30 | 10.93 | 3.44 | 0.62 | 5.56 | 2.60 |
| Tommy Toe | Feb. 24, 2010 | 6.12 | 18.58 | 21.55 | 9.50 | 3.61 | 0.61 | 5.87 | 2.63 |
| Supermarket Cherry | Feb. 24, 2010 | 5.40 | 13.10 | 17.61 | 6.29 | 4.55 | 0.33 | 13.93 | 3.09 |
| Porter | Feb. 24, 2010 | 5.28 | 17.46 | 19.68 | 13.56 | 2.27 | 0.47 | 4.79 | 1.59 |
| Matina | Mar. 2, 2010 | 5.93 | 18.40 | 21.24 | 14.53 | 2.28 | 0.44 | 5.14 | 2.15 |
| Bloody Butcher | Mar. 2, 2010 | 6.40 | 21.86 | 27.41 | 12.75 | 3.28 | 0.59 | 5.56 | 0.90 |
| Chadwick Cherry | Mar. 2, 2010 | 7.87 | 28.44 | 30.36 | 15.74 | 3.38 | 0.35 | 9.58 | 2.58 |
| Thai Pink Cherry | Mar. 2, 2010 | 4.77 | 15.39 | 17.14 | 12.89 | 2.33 | 0.19 | 12.35 | 2.06 |
| Peacevine Cherry | Mar. 2, 2010 | 7.63 | 23.65 | 26.55 | 10.20 | 4.59 | 0.33 | 13.80 | 1.66 |
| Supermarket Cherry | Mar. 2, 2010 | 5.67 | 14.70 | 18.39 | 5.47 | 5.75 | 0.30 | 19.34 | 1.69 |
| Stupice | Mar. 2, 2010 | 5.30 | 15.11 | 17.40 | 10.79 | 2.38 | 0.64 | 3.74 | 2.47 |
| Large Red Cherry | Mar. 2, 2010 | 6.10 | 17.76 | 21.00 | 9.80 | 3.50 | 0.46 | 7.62 | 3.15 |
| Bloody Butcher | Mar. 18, 2010 | 6.77 | 22.09 | 25.91 | 13.11 | 3.08 | 0.58 | 5.29 | 1.57 |
| Giant Belgium | Mar. 18, 2010 | 5.90 | 18.98 | 22.58 | 15.52 | 2.43 | 0.24 | 10.01 | 1.36 |
| Thessaloniki | Mar. 18, 2010 | 6.37 | 21.20 | 23.19 | 15.11 | 2.49 | 0.45 | 5.55 | 9.01 |
| Supermarket Round | Mar. 18, 2010 | 3.83 | 9.71 | 11.70 | 5.17 | 3.35 | 0.79 | 4.26 | 2.05 |
| Marmande VFA | Mar. 18, 2010 | 4.93 | 7.39 | 8.64 | 4.45 | 2.59 | 1.01 | 2.57 | 0.67 |
| Red Calabash | Apr. 1, 2010 | 8.53 | 29.66 | 35.78 | 9.83 | 5.83 | 0.83 | 7.05 | 1.26 |
| Brandywine | Apr. 1, 2010 | 7.00 | 25.12 | 28.90 | 9.94 | 5.03 | 0.40 | 12.59 | 1.17 |
| Dixie Golden Giant | Apr. 1, 2010 | 6.13 | 20.77 | 25.32 | 12.01 | 3.45 | 0.38 | 9.00 | 0.57 |
| Aunt Ruby's German Green | Apr. 1, 2010 | 5.80 | 21.07 | 24.51 | 9.44 | 4.47 | 0.36 | 12.32 | 1.16 |
| Thessaloniki | Apr. 1, 2010 | 5.77 | 19.82 | 23.12 | 14.93 | 2.50 | 0.38 | 6.58 | 1.47 |
| Supermarket Round | Apr. 1, 2010 | 4.20 | 11.05 | 14.14 | 5.81 | 3.83 | 0.50 | 7.60 | 1.63 |

TABLE 8E

| | | Soluble | | | | citric | malic | | glutamatic |
|---|---|---|---|---|---|---|---|---|---|
| | panel | solids | glucose | fructose | sugar:acid | acid | acid | citric:malic | acid |
| Variety | date | Brix | mg/gfw | mg/gfw | ratio | mg/gfw | mg/gfw | ratio | mg/gfw |
| Supermarket Round | Jun. 11, 2010 | 4.90 | 15.40 | 17.11 | 8.92 | 3.20 | 0.44 | 7.21 | 2.61 |
| Three Sisters | Jun. 11, 2010 | 4.70 | 13.97 | 16.20 | 6.39 | 3.80 | 0.92 | 4.12 | 1.33 |
| Skorospelka Red | Jun. 11, 2010 | 3.77 | 8.98 | 12.22 | 3.30 | 5.88 | 0.55 | 10.75 | 1.43 |
| Yellow Perfection | Jun. 11, 2010 | 4.50 | 12.22 | 15.00 | 5.18 | 3.62 | 1.64 | 2.20 | 1.35 |
| Clear Pink Slicer | Jun. 11, 2010 | 3.80 | 12.25 | 15.40 | 6.65 | 3.75 | 0.40 | 9.28 | 0.69 |
| Tigerella Orange | Jun. 11, 2010 | 3.67 | 10.59 | 13.08 | 5.60 | 3.08 | 1.15 | 2.67 | 0.70 |
| Gulf State Market | Jun. 17, 2010 | 4.90 | 14.57 | 16.09 | 6.75 | 3.98 | 0.56 | 7.11 | 1.64 |
| Bloody Butcher | Jun. 17, 2010 | 4.77 | 15.61 | 19.35 | 9.21 | 2.90 | 0.89 | 3.25 | 1.26 |
| Garden Peach | Jun. 17, 2010 | 4.20 | 12.40 | 15.32 | 5.10 | 4.86 | 0.57 | 8.49 | 1.10 |
| Amish Salad | Jun. 17, 2010 | 4.50 | 14.38 | 17.00 | 8.14 | 3.00 | 0.86 | 3.49 | 1.76 |
| Ailsa Craig | Jun. 17, 2010 | 4.63 | 13.72 | 16.24 | 5.96 | 3.95 | 1.08 | 3.67 | 1.32 |
| Supermarket Round | Jun. 17, 2010 | 4.47 | 10.11 | 15.43 | 6.82 | 3.16 | 0.59 | 5.39 | 1.95 |
| Super Sioux | Jun. 30, 2010 | 4.73 | 15.26 | 15.78 | 7.74 | 3.56 | 0.45 | 7.89 | 1.36 |
| St. Pierre | Jun. 30, 2010 | 4.53 | 14.53 | 15.76 | 9.95 | 2.21 | 0.83 | 2.67 | 1.10 |
| Supermarket Round | Jun. 30, 2010 | 4.73 | 14.57 | 16.66 | 10.23 | 2.53 | 0.52 | 4.90 | 2.14 |
| Kentucky Beefsteak | Jun. 30, 2010 | 4.50 | 14.18 | 14.98 | 9.79 | 2.66 | 0.32 | 8.28 | 1.28 |
| Green Zebra | Jun. 30, 2010 | 5.67 | 15.73 | 17.93 | 5.62 | 5.43 | 0.56 | 9.78 | 2.20 |
| Zapotec | Jun. 30, 2010 | 3.70 | 10.00 | 12.22 | 8.40 | 1.62 | 1.03 | 1.58 | 0.95 |
| Supermarket Cherry | Jul. 8, 2010 | 7.43 | 26.45 | 28.09 | 12.05 | 3.79 | 0.74 | 5.12 | 2.71 |
| Yellow Jelly Bean | Jul. 8, 2010 | 6.53 | 21.14 | 23.77 | 9.93 | 4.31 | 0.21 | 20.28 | 2.03 |
| Mexico Midget | Jul. 8, 2010 | 6.17 | 17.95 | 20.71 | 7.02 | 5.27 | 0.23 | 22.78 | 5.24 |
| Lemon Drop | Jul. 8, 2010 | 6.93 | 21.72 | 23.73 | 9.98 | 4.30 | 0.25 | 16.98 | 4.08 |
| LA1482 | Jul. 8, 2010 | 5.07 | 11.71 | 16.46 | 4.41 | 6.11 | 0.28 | 21.94 | 1.38 |
| Matt's Wild Cherry | Jul. 8, 2010 | 7.23 | 17.65 | 18.81 | 6.53 | 5.40 | 0.18 | 29.25 | 2.95 |

TABLE 8F

| | | Soluble | | | | citric | malic | | glutamatic |
|---|---|---|---|---|---|---|---|---|---|
| | panel | solids | glucose | fructose | sugar:acid | acid | acid | citric:malic | acid |
| Variety | date | Brix | mg/gfw | mg/gfw | ratio | mg/gfw | mg/gfw | ratio | mg/gfw |
| Maglia Rosa Cherry | Nov. 3, 2010 | 6.20 | 16.24 | 19.98 | 6.11 | 5.64 | 0.29 | 19.75 | 2.61 |
| Wisconsin 55 | Nov. 3, 2010 | 4.23 | 11.48 | 14.90 | 5.52 | 4.37 | 0.41 | 10.72 | 2.15 |
| Glacier | Nov. 3, 2010 | 4.40 | 9.42 | 12.98 | 4.22 | 3.97 | 1.34 | 2.97 | 1.62 |
| Oregon Spring Bush | Nov. 3, 2010 | 3.40 | 6.85 | 10.30 | 4.10 | 3.82 | 0.36 | 10.55 | 1.19 |
| Santiam | Nov. 3, 2010 | 3.53 | 6.76 | 10.46 | 3.46 | 4.56 | 0.42 | 10.73 | 1.38 |
| Giant Oxheart | Nov. 3, 2010 | 3.77 | 6.42 | 9.41 | 4.01 | 2.95 | 0.99 | 2.97 | 1.79 |
| Micado Violettor | Nov. 12, 2010 | 4.47 | 9.78 | 13.04 | 4.61 | 3.71 | 1.24 | 2.99 | 1.36 |
| Livingston's Globe | Nov. 12, 2010 | 4.63 | 12.53 | 16.73 | 7.03 | 3.83 | 0.33 | 11.57 | 1.05 |
| Old Brooks | Nov. 12, 2010 | 4.90 | 14.43 | 17.52 | 9.77 | 2.65 | 0.62 | 4.25 | 1.73 |
| Park's Whopper | Nov. 12, 2010 | 4.53 | 12.36 | 14.98 | 6.58 | 3.73 | 0.43 | 8.75 | 1.30 |
| Hillbilly | Nov. 12, 2010 | 4.07 | 10.21 | 14.57 | 8.00 | 2.29 | 0.81 | 2.84 | 1.01 |
| German Head | Nov. 12, 2010 | 4.47 | 11.13 | 14.59 | 5.59 | 4.24 | 0.36 | 11.66 | 1.08 |
| Livingston's Stone | Nov. 17, 2010 | 4.77 | 12.56 | 16.65 | 8.33 | 2.80 | 0.71 | 3.95 | 1.84 |
| Ponderosa Pink | Nov. 17, 2010 | 4.07 | 9.17 | 13.53 | 5.49 | 3.70 | 0.44 | 8.47 | 2.08 |
| Cherokee Purple | Nov. 17, 2010 | 4.27 | 10.29 | 15.59 | 6.60 | 3.42 | 0.50 | 6.87 | 1.10 |
| Black | Nov. 17, 2010 | 4.40 | 12.19 | 18.03 | 8.35 | 3.16 | 0.45 | 6.96 | 1.08 |
| Nyagous | Nov. 17, 2010 | 3.97 | 10.62 | 15.32 | 8.13 | 2.39 | 0.80 | 2.98 | 1.07 |
| Oaxacan Pink | Nov. 17, 2010 | 3.90 | 5.08 | 9.53 | 2.04 | 6.74 | 0.41 | 16.37 | 2.38 |
| Livingston's Golden Queen | Nov. 23, 2010 | 4.87 | 10.03 | 12.72 | 4.14 | 5.06 | 0.43 | 11.72 | 1.42 |
| Ponderosa Red | Nov. 23, 2010 | 4.17 | 9.35 | 13.72 | 6.00 | 3.05 | 0.80 | 3.84 | 1.34 |
| Japanese Black Trifele | Nov. 23, 2010 | 4.17 | 10.63 | 14.63 | 7.88 | 2.81 | 0.40 | 7.11 | 0.81 |
| Crimson Sprinter | Nov. 23, 2010 | 5.00 | 11.93 | 17.24 | 5.07 | 5.34 | 0.40 | 13.22 | 1.66 |
| Legend | Nov. 23, 2010 | 4.17 | 9.72 | 12.83 | 5.47 | 3.77 | 0.35 | 10.73 | 1.75 |
| Zapotec | Nov. 23, 2010 | 3.90 | 8.01 | 13.44 | 9.84 | 1.49 | 0.69 | 2.14 | 1.21 |
| Sunray | Dec. 1, 2010 | 4.50 | 9.64 | 14.33 | 5.63 | 3.90 | 0.35 | 11.01 | 1.54 |
| Martian Giant | Dec. 1, 2010 | 4.07 | 9.95 | 13.77 | 6.66 | 2.76 | 0.81 | 3.42 | 1.77 |
| Rose Tomato | Dec. 1, 2010 | 4.30 | 10.13 | 12.86 | 5.08 | 4.08 | 0.45 | 9.09 | 0.97 |
| Mr. Stripey | Dec. 1, 2010 | 4.37 | 11.45 | 16.46 | 8.83 | 2.35 | 0.81 | 2.92 | 1.22 |
| Tasti-Lee | Dec. 1, 2010 | 4.13 | 9.89 | 12.27 | 5.32 | 3.70 | 0.46 | 8.10 | 1.40 |
| Costoluto Genovese | Dec. 1, 2010 | 4.33 | 7.21 | 12.23 | 3.77 | 3.85 | 1.30 | 2.96 | 2.01 |

TABLE 8G

| | | | | | | | | | 3- |
|---|---|---|---|---|---|---|---|---|---|
| | | 1- | | | | 1- | cis-2- | | methyl- |
| | | penten- | trans-2- | 3- | 1- | penten- | penten- | isopentyl | 1- |
| | panel | 3-one | pentenal | pentanone | pentanol | 3-ol | 1-ol | acetate | pentanol |
| Variety | date | ng/gfw/hr | ng/gfw/hr | ng/gfw/hr | ng/gfw/hr | ng/gfw/hr | ng/gfw/hr | ng/gfw/hr | ng/gfw/hr |
| Cherry Roma | Feb. 24, 2010 | 3.40 | 1.27 | 12.56 | 5.58 | 4.71 | 1.69 | 0.00 | 1.37 |
| Ailsa Craig | Feb. 24, 2010 | 2.66 | 2.04 | 8.09 | 3.86 | 5.58 | 1.67 | 0.33 | 1.20 |
| Red Pear | Feb. 24, 2010 | 1.15 | 0.77 | 6.34 | 2.62 | 3.48 | 1.10 | 0.02 | 0.64 |
| Tommy Toe | Feb. 24, 2010 | 2.56 | 1.93 | 8.62 | 3.51 | 5.88 | 1.58 | 0.24 | 0.77 |
| Supermarket Cherry | Feb. 24, 2010 | 1.14 | 0.28 | 5.49 | 2.82 | 3.16 | 0.41 | 0.00 | 0.19 |
| Porter | Feb. 24, 2010 | 1.12 | 0.70 | 5.27 | 4.52 | 3.87 | 0.80 | 0.01 | 0.42 |
| Matina | Mar. 2, 2010 | 4.20 | 1.66 | 8.20 | 8.38 | 5.78 | 1.74 | 0.89 | 1.48 |
| Bloody Butcher | Mar. 2, 2010 | 1.37 | 0.98 | 7.58 | 3.46 | 5.26 | 1.04 | 0.69 | 0.74 |
| Chadwick Cherry | Mar. 2, 2010 | 3.91 | 2.98 | 8.61 | 5.66 | 6.75 | 1.34 | 0.45 | 0.24 |
| Thai Pink Cherry | Mar. 2, 2010 | 1.25 | 0.87 | 7.26 | 4.10 | 6.12 | 1.62 | 0.02 | 1.56 |
| Peacevine Cherry | Mar. 2, 2010 | 1.82 | 0.98 | 8.29 | 6.57 | 3.81 | 0.73 | 0.04 | 0.25 |
| Supermarket Cherry | Mar. 2, 2010 | 1.56 | 0.37 | 7.18 | 3.32 | 3.41 | 0.76 | 0.03 | 0.47 |
| Stupice | Mar. 2, 2010 | 1.79 | 1.79 | 8.30 | 4.24 | 6.58 | 1.78 | 0.16 | 0.88 |
| Large Red Cherry | Mar. 2, 2010 | 3.25 | 2.38 | 9.45 | 5.07 | 6.77 | 2.15 | 0.47 | 1.09 |
| Bloody Butcher | Mar. 18, 2010 | 2.29 | 1.39 | 8.87 | 3.91 | 5.87 | 0.99 | 0.47 | 0.62 |
| Giant Belgium | Mar. 18, 2010 | 2.06 | 1.63 | 9.56 | 5.04 | 6.60 | 1.16 | 0.10 | 0.36 |
| Thessaloniki | Mar. 18, 2010 | 1.26 | 0.91 | 7.62 | 3.09 | 5.81 | 1.58 | 0.55 | 1.32 |
| Supermarket Round | Mar. 18, 2010 | 0.32 | 0.16 | 2.94 | 1.77 | 3.36 | 1.46 | 0.39 | 2.27 |
| Marmande VFA | Mar. 18, 2010 | 0.38 | 0.54 | 2.80 | 2.82 | 4.51 | 1.03 | 0.02 | 0.38 |
| Red Calabash | Apr. 1, 2010 | 2.41 | 1.70 | 8.87 | 3.18 | 6.42 | 1.38 | 0.69 | 0.30 |
| Brandywine | Apr. 1, 2010 | 2.11 | 1.51 | 6.18 | 3.71 | 7.31 | 1.66 | 0.02 | 0.30 |
| Dixie Golden Giant | Apr. 1, 2010 | 0.88 | 0.59 | 4.39 | 2.18 | 5.37 | 1.12 | 0.17 | 0.32 |
| Aunt Ruby's German | Apr. 1, 2010 | 2.70 | 1.83 | 11.42 | 6.08 | 8.20 | 1.78 | 0.13 | 0.20 |
| Green | | | | | | | | | |
| Thessaloniki | Apr. 1, 2010 | 1.10 | 1.15 | 4.75 | 3.49 | 5.52 | 1.41 | 0.07 | 0.53 |
| Supermarket Round | Apr. 1, 2010 | 0.87 | 0.42 | 5.25 | 2.40 | 8.02 | 1.35 | 0.01 | 0.29 |

TABLE 8H

| | | | | | | | | | 3- |
|---|---|---|---|---|---|---|---|---|---|
| | | 1- | | | | 1- | cis-2- | | methyl- |
| | | penten- | trans-2- | 3- | 1- | penten- | penten- | isopentyl | 1- |
| | panel | 3-one | pentenal | pentanone | pentanol | 3-ol | 1-ol | acetate | pentanol |
| Variety | date | ng/gfw/hr | ng/gfw/hr | ng/gfw/hr | ng/gfw/hr | ng/gfw/hr | ng/gfw/hr | ng/gfw/hr | ng/gfw/hr |
| Supermarket Round | Jun. 11, 2010 | 0.37 | 0.26 | 2.46 | 3.54 | 2.67 | 0.79 | 0.07 | 0.76 |
| Three Sisters | Jun. 11, 2010 | 1.08 | 1.26 | 5.82 | 4.06 | 4.16 | 0.90 | 0.25 | 0.19 |
| Skorospelka Red | Jun. 11, 2010 | 0.32 | 0.83 | 3.19 | 3.02 | 2.56 | 0.87 | 0.56 | 0.52 |
| Yellow Perfection | Jun. 11, 2010 | 0.92 | 1.20 | 3.78 | 2.46 | 3.52 | 1.36 | 0.76 | 0.64 |
| Clear Pink Slicer | Jun. 11, 2010 | 0.58 | 0.83 | 2.97 | 2.58 | 2.74 | 0.78 | 0.68 | 0.26 |
| Tigerella Orange | Jun. 11, 2010 | 0.77 | 1.04 | 3.87 | 3.10 | 3.27 | 0.89 | 0.39 | 0.19 |
| Gulf State Market | Jun. 17, 2010 | 0.37 | 0.63 | 2.78 | 3.09 | 1.94 | 0.71 | 0.54 | 0.74 |
| Bloody Butcher | Jun. 17, 2010 | 0.64 | 0.99 | 3.93 | 4.24 | 2.48 | 0.49 | 0.48 | 0.34 |
| Garden Peach | Jun. 17, 2010 | 0.24 | 0.31 | 2.08 | 1.38 | 1.45 | 0.55 | 0.09 | 0.41 |
| Amish Salad | Jun. 17, 2010 | 0.39 | 0.54 | 3.27 | 3.06 | 2.53 | 0.59 | 0.12 | 0.27 |
| Ailsa Craig | Jun. 17, 2010 | 1.17 | 1.45 | 4.10 | 4.87 | 2.88 | 0.68 | 0.52 | 0.21 |
| Supermarket Round | Jun. 17, 2010 | 0.61 | 0.19 | 2.93 | 4.30 | 3.99 | 0.83 | 0.41 | 0.55 |
| Super Sioux | Jun. 30, 2010 | 0.50 | 0.66 | 4.18 | 2.84 | 2.25 | 0.56 | 0.08 | 0.24 |
| St. Pierre | Jun. 30, 2010 | 0.85 | 1.02 | 3.62 | 3.15 | 3.10 | 0.57 | 0.08 | 0.10 |
| Supermarket Round | Jun. 30, 2010 | 0.51 | 0.31 | 2.81 | 2.04 | 3.08 | 0.61 | 0.07 | 0.17 |
| Kentucky Beefsteak | Jun. 30, 2010 | 0.73 | 0.47 | 3.29 | 1.77 | 2.94 | 0.38 | 0.01 | 0.11 |
| Green Zebra | Jun. 30, 2010 | 1.12 | 1.60 | 6.67 | 3.23 | 3.59 | 1.20 | 0.16 | 0.74 |
| Zapotec | Jun. 30, 2010 | 0.31 | 0.44 | 2.72 | 1.60 | 2.28 | 0.35 | 0.04 | 0.10 |
| Supermarket Cherry | Jul. 8, 2010 | 1.05 | 0.34 | 4.65 | 4.00 | 2.50 | 0.55 | 0.00 | 0.17 |
| Yellow Jelly Bean | Jul. 8, 2010 | 0.82 | 0.74 | 3.26 | 1.98 | 1.66 | 0.43 | 0.02 | 0.20 |
| Mexico Midget | Jul. 8, 2010 | 0.89 | 0.61 | 4.34 | 2.57 | 1.88 | 0.96 | 0.03 | 0.52 |
| Lemon Drop | Jul. 8, 2010 | 0.71 | 0.45 | 3.25 | 2.39 | 1.96 | 0.46 | 0.24 | 0.29 |
| LA1482 | Jul. 8, 2010 | 0.55 | 1.59 | 3.71 | 2.92 | 4.06 | 0.55 | 0.12 | 0.02 |
| Matt's Wild Cherry | Jul. 8, 2010 | 0.38 | 0.31 | 1.46 | 2.55 | 1.76 | 0.36 | 0.03 | 0.18 |

TABLE 8I

| | | 1-penten-3- | trans-2- | 3- | 1- | 1-penten- | cis-2- | isopentyl | 3-methyl-1- |
|---|---|---|---|---|---|---|---|---|---|
| | | one | pentenal | pentanone | pentanol | 3-ol | penten-1-ol | acetate | pentanol |
| Variety | panel date | ng/gfw/hr | ng/gfw/hr | ng/gfw/hr | ng/gfw/hr | ng/gfw/hr | ng/gfw/hr | ng/gfw/hr | ng/gfw/hr |
| Maglia Rosa Cherry | Nov. 3, 2010 | 0.84 | 1.77 | 7.45 | 3.69 | 6.06 | 2.31 | 0.10 | 2.19 |
| Wisconsin 55 | Nov. 3, 2010 | 0.69 | 1.14 | 4.47 | 4.60 | 4.82 | 2.50 | 0.99 | 2.79 |
| Glacier | Nov. 3, 2010 | 0.61 | 0.84 | 5.22 | 2.61 | 3.99 | 1.12 | 0.37 | 0.92 |
| Oregon Spring Bush | Nov. 3, 2010 | 0.40 | 0.61 | 3.85 | 2.64 | 3.71 | 1.00 | 0.24 | 0.75 |
| Santiam | Nov. 3, 2010 | 0.57 | 0.97 | 4.53 | 2.54 | 3.62 | 1.36 | 0.28 | 1.11 |
| Giant Oxheart | Nov. 3, 2010 | 0.69 | 1.23 | 4.93 | 3.77 | 4.84 | 2.15 | 0.26 | 1.81 |
| Micado Violettor | Nov. 12, 2010 | 1.40 | 0.57 | 7.49 | 1.74 | 3.47 | 0.90 | 0.07 | 0.11 |
| Livingston's Globe | Nov. 12, 2010 | 0.89 | 0.66 | 5.71 | 1.60 | 5.22 | 1.15 | 0.11 | 0.23 |
| Old Brooks | Nov. 12, 2010 | 1.91 | 1.25 | 5.65 | 2.50 | 6.11 | 1.77 | 0.13 | 0.52 |
| Park's Whopper | Nov. 12, 2010 | 0.95 | 0.70 | 7.83 | 2.63 | 4.78 | 1.00 | 0.17 | 0.33 |
| Hillbilly | Nov. 12, 2010 | 1.98 | 0.85 | 6.94 | 1.80 | 4.57 | 1.47 | 0.11 | 0.62 |
| German Head | Nov. 12, 2010 | 0.80 | 0.48 | 7.40 | 2.71 | 4.14 | 1.08 | 0.11 | 0.64 |
| Livingston's Stone | Nov. 17, 2010 | 0.69 | 0.65 | 5.89 | 2.23 | 4.08 | 0.56 | 0.17 | 0.16 |
| Ponderosa Pink | Nov. 17, 2010 | 0.53 | 0.56 | 5.10 | 2.35 | 5.70 | 1.52 | 0.37 | 1.32 |
| Cherokee Purple | Nov. 17, 2010 | 0.72 | 0.46 | 9.04 | 4.69 | 3.86 | 1.00 | 0.44 | 0.65 |
| Black | Nov. 17, 2010 | 0.63 | 0.37 | 6.41 | 3.85 | 3.89 | 0.76 | 0.06 | 0.25 |
| Nyagous | Nov. 17, 2010 | 1.10 | 0.73 | 8.99 | 4.70 | 3.70 | 0.95 | 0.20 | 0.33 |
| Oaxacan Pink | Nov. 17, 2010 | 1.11 | 0.67 | 8.64 | 3.23 | 6.22 | 1.91 | 1.32 | 1.60 |
| Livingston's Golden Queen | Nov. 23, 2010 | 1.60 | 0.99 | 11.67 | 2.48 | 7.10 | 1.67 | 0.37 | 0.40 |
| Ponderosa Red | Nov. 23, 2010 | 0.46 | 0.31 | 5.14 | 1.59 | 3.74 | 0.73 | 0.27 | 0.30 |
| Japanese Black Trifele | Nov. 23, 2010 | 0.23 | 0.13 | 3.88 | 2.80 | 2.64 | 0.44 | 0.06 | 0.10 |
| Crimson Sprinter | Nov. 23, 2010 | 0.75 | 0.69 | 8.07 | 2.18 | 5.11 | 1.40 | 0.30 | 0.83 |
| Legend | Nov. 23, 2010 | 0.73 | 0.40 | 7.86 | 2.10 | 5.00 | 1.35 | 0.22 | 0.91 |
| Zapotec | Nov. 23, 2010 | 0.37 | 0.37 | 4.14 | 1.86 | 4.58 | 0.65 | 0.12 | 0.12 |
| Sunray | Dec. 1, 2010 | 0.60 | 0.31 | 5.38 | 1.39 | 3.55 | 1.91 | 0.34 | 2.11 |
| Martian Giant | Dec. 1, 2010 | 0.58 | 0.38 | 6.75 | 1.50 | 4.17 | 1.74 | 0.37 | 1.34 |
| Rose Tomato | Dec. 1, 2010 | 1.21 | 0.60 | 7.84 | 2.64 | 5.63 | 1.57 | 0.17 | 0.81 |
| Mr. Stripey | Dec. 1, 2010 | 0.50 | 0.20 | 4.63 | 1.09 | 2.27 | 1.32 | 0.27 | 0.69 |
| Tasti-Lee | Dec. 1, 2010 | 0.62 | 0.44 | 6.61 | 1.52 | 3.83 | 1.17 | 0.28 | 0.72 |
| Costoluto Genovese | Dec. 1, 2010 | 0.77 | 0.58 | 7.62 | 1.81 | 4.88 | 1.24 | 0.19 | 0.41 |

TABLE 8J

| | | | | cis-3- | | | trans-3- | | cis-3- | |
|---|---|---|---|---|---|---|---|---|---|---|
| | | hexyl | | hexen-1- | cis-3- | trans-2- | hexen-1- | hexyl | hexenyl | trans-2- |
| | panel | alcohol | hexanal | ol | hexenal | hexenal | ol | acetate | acetate | heptenal |
| Variety | date | ng/gfw/hr | ng/gfw/hr | ng/gfw/hr | ng/gfw/hr | ng/gfw/hr | ng/gfw/hr | ng/gfw/hr | ng/gfw/hr | ng/gfw/hr |
| Cherry Roma | Feb. 24, 2010 | 8.95 | 68.39 | 34.74 | 52.68 | 1.68 | 2.19 | 0.16 | 2.37 | 0.71 |
| Ailsa Craig | Feb. 24, 2010 | 19.14 | 164.00 | 47.06 | 168.52 | 9.91 | 1.71 | 0.22 | 1.34 | 0.64 |
| Red Pear | Feb. 24, 2010 | 11.60 | 127.50 | 26.96 | 81.69 | 3.04 | 2.03 | 0.15 | 0.85 | 0.28 |
| Tommy Toe | Feb. 24, 2010 | 12.43 | 128.81 | 42.78 | 157.39 | 6.13 | 2.45 | 0.24 | 1.26 | 0.58 |
| Supermarket Cherry | Feb. 24, 2010 | 11.60 | 49.59 | 23.37 | 29.09 | 0.38 | 0.53 | 0.01 | 0.68 | 0.15 |
| Porter | Feb. 24, 2010 | 31.12 | 95.45 | 42.19 | 39.58 | 1.41 | 0.96 | 0.13 | 1.54 | 0.41 |
| Matina | Mar. 2, 2010 | 34.88 | 107.77 | 46.11 | 42.78 | 5.67 | 1.87 | 0.43 | 1.87 | 1.21 |
| Bloody Butcher | Mar. 2, 2010 | 16.95 | 93.94 | 47.09 | 60.98 | 1.88 | 1.42 | 0.47 | 1.68 | 0.22 |
| Chadwick Cherry | Mar. 2, 2010 | 69.30 | 173.87 | 103.87 | 112.85 | 12.29 | 1.93 | 1.03 | 2.26 | 1.14 |
| Thai Pink Cherry | Mar. 2, 2010 | 17.64 | 144.84 | 36.09 | 70.78 | 2.50 | 1.28 | 0.16 | 1.39 | 0.38 |
| Peacevine Cherry | Mar. 2, 2010 | 13.72 | 76.15 | 30.72 | 34.73 | 0.88 | 1.12 | 0.45 | 2.26 | 0.93 |
| Supermarket Cherry | Mar. 2, 2010 | 7.79 | 34.94 | 28.32 | 32.84 | 0.81 | 0.60 | 0.02 | 0.73 | 0.13 |
| Stupice | Mar. 2, 2010 | 22.57 | 208.78 | 40.61 | 143.68 | 7.55 | 1.85 | 0.34 | 1.66 | 0.67 |
| Large Red Cherry | Mar. 2, 2010 | 20.70 | 212.58 | 71.57 | 198.26 | 9.81 | 3.40 | 0.22 | 1.59 | 0.84 |
| Bloody Butcher | Mar. 18, 2010 | 29.71 | 116.54 | 40.15 | 49.01 | 2.89 | 1.00 | 0.50 | 1.67 | 0.37 |
| Giant Belgium | Mar. 18, 2010 | 48.83 | 306.77 | 48.53 | 36.79 | 9.92 | 1.29 | 0.53 | 2.56 | 0.58 |
| Thessaloniki | Mar. 18, 2010 | 13.30 | 138.32 | 30.05 | 61.95 | 2.37 | 3.37 | 0.22 | 1.65 | 0.20 |
| Supermarket Round | Mar. 18, 2010 | 17.33 | 147.30 | 28.09 | 69.68 | 1.11 | 0.73 | 0.53 | 1.05 | 0.12 |
| Marmande VFA | Mar. 18, 2010 | 19.04 | 164.65 | 33.02 | 81.83 | 1.88 | 0.59 | 0.99 | 0.74 | 0.15 |
| Red Calabash | Apr. 1, 2010 | 9.17 | 131.25 | 49.44 | 175.07 | 11.40 | 2.24 | 0.32 | 1.36 | 0.45 |
| Brandywine | Apr. 1, 2010 | 17.27 | 143.18 | 45.87 | 161.32 | 6.70 | 0.68 | 0.66 | 1.59 | 0.56 |
| Dixie Golden Giant | Apr. 1, 2010 | 5.71 | 82.28 | 19.22 | 79.24 | 5.30 | 0.28 | 0.80 | 1.87 | 0.24 |
| Aunt Ruby's German | Apr. 1, 2010 | 25.14 | 162.76 | 59.85 | 131.33 | 7.25 | 1.65 | 0.38 | 1.52 | 0.62 |
| Green | | | | | | | | | | |
| Thessaloniki | Apr. 1, 2010 | 22.34 | 124.72 | 38.50 | 50.64 | 2.87 | 2.62 | 0.12 | 0.84 | 0.20 |
| Supermarket Round | Apr. 1, 2010 | 15.60 | 68.08 | 36.48 | 44.21 | 0.72 | 0.64 | 0.19 | 0.82 | 0.11 |

TABLE 8K

| | | | | cis-3- | | | trans-3- | | cis-3- | |
|---|---|---|---|---|---|---|---|---|---|---|
| | | hexyl | | hexen-1- | cis-3- | trans-2- | hexen-1- | hexyl | hexenyl | trans-2- |
| | panel | alcohol | hexanel | ol | hexenal | hexenal | ol | acetate | acetate | heptenal |
| Variety | date | ng/gfw/hr | ng/gfw/hr | ng/gfw/hr | ng/gfw/hr | ng/gfw/hr | ng/gfw/hr | ng/gfw/hr | ng/gfw/hr | ng/gfw/hr |
| Supermarket Round | Jun. 11, 2010 | 49.77 | 105.96 | 42.45 | 22.28 | 2.75 | 0.83 | 1.15 | 2.03 | 0.28 |
| Three Sisters | Jun. 11, 2010 | 32.87 | 85.31 | 49.30 | 52.63 | 5.73 | 1.29 | 0.57 | 1.89 | 0.60 |
| Skorospelka Red | Jun. 11, 2010 | 35.22 | 135.81 | 52.41 | 68.99 | 2.67 | 1.49 | 0.55 | 1.51 | 0.42 |
| Yellow Perfection | Jun. 11, 2010 | 8.78 | 96.66 | 42.54 | 158.23 | 4.83 | 1.52 | 0.18 | 1.73 | 0.29 |
| Clear Pink Slicer | Jun. 11, 2010 | 25.85 | 132.11 | 49.81 | 84.45 | 3.71 | 0.92 | 0.68 | 2.15 | 0.32 |
| Tigerella Orange | Jun. 11, 2010 | 22.59 | 86.12 | 62.93 | 88.72 | 6.71 | 0.74 | 0.49 | 2.47 | 0.26 |
| Gulf State Market | Jun. 17, 2010 | 9.16 | 52.21 | 24.66 | 34.45 | 2.53 | 1.39 | 0.26 | 1.42 | 0.27 |
| Bloody Butcher | Jun. 17, 2010 | 27.83 | 44.75 | 34.83 | 19.56 | 6.37 | 0.69 | 0.66 | 2.03 | 0.44 |
| Garden Peach | Jun. 17, 2010 | 2.99 | 22.12 | 11.08 | 21.44 | 0.82 | 0.50 | 0.07 | 0.59 | 0.08 |
| Amish Salad | Jun. 17, 2010 | 10.30 | 47.11 | 24.30 | 27.19 | 1.29 | 0.81 | 0.17 | 1.31 | 0.16 |
| Ailsa Craig | Jun. 17, 2010 | 34.43 | 65.54 | 56.68 | 33.63 | 9.92 | 0.67 | 0.69 | 2.46 | 0.69 |
| Supermarket Round | Jun. 17, 2010 | 37.70 | 41.88 | 40.28 | 14.90 | 9.58 | 0.79 | 2.03 | 4.17 | 0.18 |
| Super Sioux | Jun. 30, 2010 | 15.23 | 93.58 | 29.10 | 43.14 | 1.39 | 0.93 | 0.25 | 1.19 | 0.28 |
| St. Pierre | Jun. 30, 2010 | 28.72 | 225.94 | 35.07 | 61.74 | 2.39 | 0.47 | 0.74 | 1.51 | 0.57 |
| Supermarket Round | Jun. 30, 2010 | 18.74 | 132.87 | 25.40 | 43.00 | 0.73 | 0.52 | 0.41 | 1.21 | 0.14 |
| Kentucky Beefsteak | Jun. 30, 2010 | 5.60 | 21.92 | 17.75 | 15.96 | 0.38 | 0.21 | 0.10 | 0.75 | 0.16 |
| Green Zebra | Jun. 30, 2010 | 11.29 | 136.00 | 35.39 | 122.97 | 9.29 | 1.00 | 0.20 | 1.08 | 0.38 |
| Zapotec | Jun. 30, 2010 | 12.46 | 91.85 | 19.98 | 32.72 | 1.30 | 0.35 | 0.26 | 0.84 | 0.15 |
| Supermarket Cherry | Jul. 8, 2010 | 13.48 | 45.51 | 23.94 | 23.29 | 0.29 | 0.74 | 0.04 | 1.21 | 0.32 |
| Yellow Jelly Bean | Jul. 8, 2010 | 9.94 | 41.70 | 28.50 | 31.82 | 0.91 | 1.25 | 0.08 | 0.66 | 0.16 |
| Mexico Midget | Jul. 8, 2010 | 14.33 | 196.76 | 20.87 | 102.57 | 3.42 | 1.56 | 0.13 | 0.89 | 0.43 |
| Lemon Drop | Jul. 8, 2010 | 8.50 | 26.42 | 19.44 | 14.76 | 0.37 | 1.54 | 0.62 | 0.75 | 0.16 |
| LA1482 | Jul. 8, 2010 | 39.12 | 200.77 | 35.83 | 64.16 | 27.47 | 0.40 | 0.85 | 1.62 | 0.82 |
| Matt's Wild Cherry | Jul. 8, 2010 | 21.07 | 18.65 | 27.35 | 6.87 | 0.53 | 0.79 | 0.11 | 0.53 | 0.23 |

TABLE 8L

| | | hexyl | | cis-3- | cis-3- | trans-2- | trans-3- | hexyl | cis-3- | trans-2- |
|---|---|---|---|---|---|---|---|---|---|---|
| | | alcohol | Hexane | hexen-1- | hexenal | hexenal | hexen-1-ol | acetate | hexenyl | heptenal |
| | | (ng/gfw/ | (ng/gfw/ | ol | (ng/gfw/ | (ng/gfw/ | (ng/gfw/ | (ng/gfw/ | acetate | (ng/gfw/ |
| Variety | panel date | hr) | hr) | (ng/gfw/hr) | hr) | hr) | hr) | hr) | (ng/gfw/hr) | hr) |
| Maglia Rosa Cherry | Nov. 3, 2010 | 10.27 | 97.24 | 45.54 | 151.03 | 5.43 | 1.36 | 0.29 | 1.26 | 0.235 |
| Wisconsin 55 | Nov. 3, 2010 | 17.36 | 75.76 | 58.56 | 72.48 | 5.23 | 2.50 | 0.58 | 2.43 | 0.231 |
| Glacier | Nov. 3, 2010 | 6.91 | 53.17 | 26.29 | 69.54 | 2.87 | 0.94 | 0.33 | 1.76 | 0.112 |
| Oregon Spring Bush | Nov. 3, 2010 | 8.10 | 60.03 | 33.06 | 65.84 | 2.14 | 1.07 | 0.27 | 1.94 | 0.116 |
| Santiam | Nov. 3, 2010 | 5.97 | 64.34 | 29.73 | 85.30 | 2.71 | 0.88 | 0.31 | 2.18 | 0.174 |
| Giant Oxheart | Nov. 3, 2010 | 16.19 | 77.47 | 40.16 | 51.00 | 2.27 | 0.74 | 0.89 | 2.88 | 0.254 |
| Micado Violettor | Nov. 12, 2010 | 2.07 | 46.30 | 11.63 | 63.21 | 1.31 | 0.42 | 0.13 | 1.03 | 0.099 |
| Livingston's Globe | Nov. 12, 2010 | 4.60 | 78.60 | 23.00 | 109.04 | 1.95 | 0.50 | 0.12 | 1.09 | 0.088 |
| Old Brooks | Nov. 12, 2010 | 10.26 | 134.43 | 41.83 | 197.28 | 7.57 | 0.78 | 0.25 | 1.85 | 0.258 |
| Park's Whopper | Nov. 12, 2010 | 20.56 | 140.31 | 39.40 | 90.74 | 3.20 | 0.86 | 0.88 | 2.52 | 0.138 |
| Hillbilly | Nov. 12, 2010 | 6.75 | 87.00 | 32.29 | 147.53 | 4.98 | 0.85 | 0.18 | 1.20 | 0.142 |
| German Head | Nov. 12, 2010 | 10.40 | 47.87 | 25.49 | 35.34 | 1.42 | 0.72 | 0.41 | 1.27 | 0.107 |
| Livingston's Stone | Nov. 17, 2010 | 9.12 | 55.12 | 23.26 | 38.82 | 1.46 | 0.56 | 0.36 | 1.72 | 0.087 |
| Ponderosa Pink | Nov. 17, 2010 | 15.66 | 63.99 | 34.18 | 40.13 | 2.57 | 0.92 | 0.32 | 1.15 | 0.113 |
| Cherokee Purple | Nov. 17, 2010 | 8.47 | 40.34 | 26.52 | 42.97 | 0.99 | 0.64 | 0.43 | 1.99 | 0.104 |
| Black | Nov. 17, 2010 | 9.55 | 22.88 | 31.23 | 25.10 | 0.96 | 0.36 | 0.27 | 1.22 | 0.071 |
| Nyagous | Nov. 17, 2010 | 6.47 | 29.96 | 27.01 | 37.07 | 1.26 | 0.33 | 0.21 | 1.98 | 0.167 |
| Oaxacan Pink | Nov. 17, 2010 | 19.38 | 59.23 | 40.60 | 76.68 | 4.87 | 0.92 | 0.30 | 1.46 | 0.330 |
| Livingston's Golden | Nov. 23, 2010 | 6.23 | 133.46 | 15.14 | 94.34 | 6.75 | 0.71 | 0.38 | 1.73 | 0.175 |
| Queen | | | | | | | | | | |
| Ponderosa Red | Nov. 23, 2010 | 10.58 | 83.83 | 22.55 | 49.99 | 1.45 | 0.57 | 0.73 | 1.80 | 0.045 |
| Japanese Black | Nov. 23, 2010 | 4.45 | 13.52 | 14.33 | 10.87 | 0.35 | 0.17 | 0.22 | 1.19 | 0.036 |
| Trifele | | | | | | | | | | |
| Crimson Sprinter | Nov. 23, 2010 | 9.04 | 103.88 | 27.96 | 103.17 | 3.00 | 0.94 | 0.62 | 2.32 | 0.131 |
| Legend | Nov. 23, 2010 | 10.19 | 95.45 | 28.49 | 69.76 | 1.40 | 0.90 | 0.50 | 2.05 | 0.065 |
| Zapotec | Nov. 23, 2010 | 11.52 | 57.15 | 23.86 | 31.56 | 1.86 | 0.59 | 0.26 | 0.96 | 0.044 |
| Sunray | Dec. 1, 2010 | 3.29 | 40.82 | 13.19 | 44.06 | 0.74 | 0.72 | 0.19 | 0.94 | 0.037 |
| Martian Giant | Dec. 1, 2010 | 6.93 | 99.63 | 19.40 | 76.13 | 1.50 | 0.86 | 0.42 | 1.43 | 0.067 |
| Rose Tomato | Dec. 1, 2010 | 15.74 | 146.03 | 29.59 | 124.47 | 3.87 | 0.64 | 1.30 | 1.97 | 0.137 |
| Mr. Stripey | Dec. 1, 2010 | 1.53 | 21.15 | 5.98 | 23.27 | 0.64 | 0.30 | 0.10 | 0.73 | 0.034 |
| Tasti-Lee | Dec. 1, 2010 | 7.69 | 70.62 | 18.62 | 51.17 | 1.00 | 0.41 | 0.49 | 1.46 | 0.053 |
| Costoluto Genovese | Dec. 1, 2010 | 8.92 | 177.45 | 24.82 | 158.96 | 2.57 | 0.98 | 0.66 | 1.77 | 0.098 |

TABLE 8M

| | | | | 3- | 3- | | | |
|---|---|---|---|---|---|---|---|---|
| | | | | methyl- | methyl- | | | |
| | | | | 2- | 1- | isovaleric | isobutyl | 1-nitro-3- |
| | panel | isovaleronitrile | isovaleraldehyde | butenal | butanol | acid | acetate | methylbutane |
| Variety | date | ng/gfw/hr | ng/gfw/hr | ng/gfw/hr | ng/gfw/hr | ng/gfw/hr | ng/gfw/hr | ng/gfw/hr |
| Cherry Roma | Feb. 24, 2010 | 37.73 | 21.12 | 0.34 | 44.34 | 0.239 | 0.36 | 13.29 |
| Ailsa Craig | Feb. 24, 2010 | 18.44 | 24.62 | 0.49 | 77.77 | 0.118 | 0.86 | 26.60 |
| Red Pear | Feb. 24, 2010 | 20.47 | 23.81 | 0.41 | 34.12 | 0.146 | 0.29 | 19.96 |
| Tommy Toe | Feb. 24, 2010 | 25.90 | 25.86 | 0.43 | 64.61 | 0.221 | 0.57 | 20.34 |
| Supermarket Cherry | Feb. 24, 2010 | 3.04 | 3.38 | 0.15 | 5.06 | 0.008 | 0.17 | 0.75 |
| Porter | Feb. 24, 2010 | 7.34 | 5.17 | 0.22 | 11.15 | 0.057 | 0.13 | 3.96 |
| Matina | Mar. 2, 2010 | 30.03 | 21.01 | 0.60 | 120.42 | 0.419 | 0.65 | 48.78 |
| Bloody Butcher | Mar. 2, 2010 | 8.64 | 24.49 | 0.42 | 50.45 | 0.175 | 0.56 | 18.15 |
| Chadwick Cherry | Mar. 2, 2010 | 27.30 | 12.45 | 0.51 | 33.81 | 0.044 | 0.53 | 24.27 |
| Thai Pink Cherry | Mar. 2, 2010 | 14.52 | 15.76 | 0.32 | 18.67 | 0.220 | 0.17 | 27.43 |
| Peacevine Cherry | Mar. 2, 2010 | 20.64 | 11.82 | 0.33 | 40.35 | 0.041 | 0.63 | 22.74 |
| Supermarket Cherry | Mar. 2, 2010 | 4.81 | 14.31 | 0.24 | 24.75 | 0.022 | 0.84 | 1.93 |
| Stupice | Mar. 2, 2010 | 26.64 | 15.14 | 0.38 | 33.54 | 0.173 | 0.75 | 47.77 |
| Large Red Cherry | Mar. 2, 2010 | 49.72 | 30.99 | 0.58 | 108.66 | 0.429 | 0.67 | 52.54 |
| Bloody Butcher | Mar. 18, 2010 | 30.89 | 22.42 | 0.35 | 41.50 | 0.097 | 0.38 | 55.18 |
| Giant Belgium | Mar. 18, 2010 | 13.78 | 23.02 | 0.93 | 45.82 | 0.150 | 0.60 | 35.30 |
| Thessaloniki | Mar. 18, 2010 | 38.59 | 59.18 | 0.56 | 98.12 | 0.175 | 0.61 | 88.06 |
| Supermarket Round | Mar. 18, 2010 | 8.98 | 15.62 | 0.22 | 28.81 | 0.029 | 3.06 | 25.40 |
| Marmande VFA | Mar. 18, 2010 | 10.73 | 5.73 | 0.32 | 12.81 | 0.120 | 0.42 | 6.13 |
| Red Calabash | Apr. 1, 2010 | 20.52 | 29.96 | 0.58 | 97.05 | 0.103 | 0.96 | 39.76 |
| Brandywine | Apr. 1, 2010 | 1.44 | 3.71 | 0.34 | 9.52 | 0.084 | 0.24 | 2.21 |
| Dixie Golden Giant | Apr. 1, 2010 | 1.18 | 4.72 | 0.31 | 7.89 | 0.023 | 0.71 | 0.92 |
| Aunt Ruby's German Green | Apr. 1, 2010 | 25.24 | 11.89 | 0.31 | 37.23 | 0.165 | 1.24 | 36.92 |
| Thessaloniki | Apr. 1, 2010 | 58.16 | 29.63 | 0.53 | 108.50 | 0.112 | 0.43 | 103.97 |
| Supermarket Round | Apr. 1, 2010 | 2.45 | 1.95 | 0.16 | 4.51 | 0.003 | 0.40 | 4.40 |

TABLE 8N

| | | | | 3- | 3- | | | |
|---|---|---|---|---|---|---|---|---|
| | | | | methyl- | methyl- | | | |
| | | | | 2- | 1- | isovaleric | isobutyl | 1-nitro-3- |
| | panel | isovaleronitrile | isovaleraldehyde | butenal | butanol | acid | acetate | methylbutane |
| Variety | date | ng/gfw/hr | ng/gfw/hr | ng/gfw/hr | ng/gfw/hr | ng/gfw/hr | ng/gfw/hr | ng/gfw/hr |
| Supermarket Round | Jun. 11, 2010 | 7.42 | 1.81 | 0.15 | 7.00 | 0.046 | 1.19 | 16.31 |
| Three Sisters | Jun. 11, 2010 | 14.94 | 6.84 | 0.79 | 28.83 | 0.044 | 2.14 | 17.59 |
| Skorospelka Red | Jun. 11, 2010 | 7.31 | 11.09 | 0.38 | 53.17 | 0.047 | 1.50 | 16.39 |
| Yellow Perfection | Jun. 11, 2010 | 6.89 | 14.19 | 0.26 | 62.38 | 0.053 | 2.90 | 14.58 |
| Clear Pink Slicer | Jun. 11, 2010 | 4.87 | 8.35 | 0.29 | 32.32 | 0.053 | 4.58 | 6.63 |
| Tigerella Orange | Jun. 11, 2010 | 1.92 | 3.38 | 0.20 | 17.13 | 0.053 | 1.09 | 3.12 |
| Gulf State Market | Jun. 17, 2010 | 4.63 | 6.14 | 0.36 | 44.38 | 0.017 | 3.04 | 9.16 |
| Bloody Butcher | Jun. 17, 2010 | 2.29 | 3.61 | 0.17 | 18.25 | 0.013 | 0.43 | 1.06 |
| Garden Peach | Jun. 17, 2010 | 1.08 | 3.93 | 0.17 | 16.48 | 0.010 | 0.71 | 1.53 |
| Amish Salad | Jun. 17, 2010 | 2.15 | 6.60 | 0.23 | 18.20 | 0.013 | 0.21 | 2.00 |
| Ailsa Craig | Jun. 17, 2010 | 1.76 | 4.50 | 0.20 | 23.77 | 0.017 | 0.86 | 2.20 |
| Supermarket Round | Jun. 17, 2010 | 2.40 | 3.46 | 2.25 | 15.40 | 0.012 | 7.09 | 4.06 |
| Super Sioux | Jun. 30, 2010 | 6.80 | 7.85 | 0.20 | 21.85 | 0.011 | 1.71 | 9.45 |
| St. Pierre | Jun. 30, 2010 | 4.20 | 3.84 | 0.11 | 10.55 | 0.014 | 1.03 | 2.12 |
| Supermarket Round | Jun. 30, 2010 | 7.24 | 6.00 | 0.07 | 7.75 | 0.016 | 0.42 | 12.32 |
| Kentucky Beefsteak | Jun. 30, 2010 | 0.89 | 1.09 | 0.13 | 2.51 | 0.015 | 0.22 | 0.68 |
| Green Zebra | Jun. 30, 2010 | 9.29 | 15.20 | 0.25 | 41.34 | 0.015 | 0.76 | 9.04 |
| Zapotec | Jun. 30, 2010 | 1.13 | 4.91 | 0.11 | 10.22 | 0.016 | 1.94 | 1.51 |
| Supermarket Cherry | Jul. 8, 2010 | 5.68 | 3.63 | 0.17 | 9.66 | 0.012 | 0.75 | 2.69 |
| Yellow Jelly Bean | Jul. 8, 2010 | 23.42 | 14.57 | 0.08 | 36.66 | 0.005 | 0.55 | 14.24 |
| Mexico Midget | Jul. 8, 2010 | 25.50 | 33.93 | 0.14 | 84.99 | 0.006 | 0.68 | 34.82 |
| Lemon Drop | Jul. 8, 2010 | 20.39 | 19.34 | 0.26 | 45.04 | 0.008 | 0.69 | 12.36 |
| LA1482 | Jul. 8, 2010 | 2.66 | 5.99 | 0.09 | 12.03 | 0.006 | 1.27 | 2.70 |
| Matt's Wild Cherry | Jul. 8, 2010 | 11.92 | 5.75 | 0.75 | 46.13 | 0.007 | 0.35 | 10.91 |

TABLE 8O

| | | | | 3-methyl- | 3-methyl- | isovaleric | isobutyl | 1-nitro-3- |
|---|---|---|---|---|---|---|---|---|
| | | isovaleronitrile | isovaleraldehyde | 2-butenal | 1-butanol | acid | acetate | methylbutane |
| Variety | panel date | ng/gfw/hr | ng/gfw/hr | ng/gfw/hr | ng/gfw/hr | ng/gfw/hr | ng/gfw/hr | ng/gfw/hr |
| Maglia Rosa Cherry | Nov. 3, 2010 | 3.61 | 4.94 | 0.26 | 18.23 | 0.046 | 1.40 | 3.31 |
| Wisconsin 55 | Nov. 3, 2010 | 12.73 | 18.24 | 0.67 | 94.88 | 0.042 | 3.68 | 27.75 |
| Glacier | Nov. 3, 2010 | 1.36 | 6.23 | 0.27 | 15.83 | 0.033 | 1.37 | 1.36 |
| Oregon Spring Bush | Nov. 3, 2010 | 2.37 | 7.37 | 0.28 | 16.48 | 0.028 | 1.16 | 4.06 |
| Santiam | Nov. 3, 2010 | 1.42 | 4.90 | 0.38 | 13.73 | 0.043 | 1.46 | 1.99 |
| Giant Oxheart | Nov. 3, 2010 | 2.59 | 4.73 | 0.41 | 14.21 | 0.017 | 0.98 | 3.27 |
| Micado Violettor | Nov. 12, 2010 | 2.66 | 8.05 | 0.40 | 9.17 | 0.023 | 1.32 | 2.37 |
| Livingston's Globe | Nov. 12, 2010 | 1.93 | 7.66 | 0.26 | 12.72 | 0.045 | 0.84 | 3.12 |
| Old Brooks | Nov. 12, 2010 | 11.59 | 10.76 | 0.47 | 18.32 | 0.068 | 0.55 | 20.24 |
| Park's Whopper | Nov. 12, 2010 | 1.27 | 7.05 | 0.24 | 10.80 | 0.020 | 1.47 | 1.59 |
| Hillbilly | Nov. 12, 2010 | 5.13 | 10.36 | 0.22 | 18.23 | 0.056 | 0.91 | 8.53 |
| German Head | Nov. 12, 2010 | 2.73 | 11.27 | 0.28 | 18.99 | 0.027 | 0.81 | 6.52 |
| Livingston's Stone | Nov. 17, 2010 | 2.35 | 6.37 | 0.38 | 11.48 | 0.017 | 0.68 | 1.52 |
| Ponderosa Pink | Nov. 17, 2010 | 3.08 | 9.69 | 0.67 | 24.42 | 0.035 | 2.41 | 5.80 |
| Cherokee Purple | Nov. 17, 2010 | 2.73 | 11.46 | 0.33 | 22.47 | 0.026 | 0.88 | 3.48 |
| Black | Nov. 17, 2010 | 1.64 | 3.99 | 0.26 | 9.03 | 0.013 | 0.51 | 2.20 |
| Nyagous | Nov. 17, 2010 | 1.50 | 5.45 | 0.27 | 10.48 | 0.016 | 0.74 | 1.34 |
| Oaxacan Pink | Nov. 17, 2010 | 8.05 | 10.83 | 0.41 | 64.19 | 0.111 | 1.36 | 12.10 |
| Livingston's Golden Queen | Nov. 23, 2010 | 3.69 | 18.19 | 0.18 | 21.25 | 0.035 | 0.59 | 5.14 |
| Ponderosa Red | Nov. 23, 2010 | 2.63 | 7.02 | 0.28 | 9.77 | 0.027 | 2.64 | 1.74 |
| Japanese Black Trifele | Nov. 23, 2010 | 0.73 | 1.57 | 0.17 | 2.77 | 0.012 | 0.69 | 0.55 |
| Crimson Sprinter | Nov. 23, 2010 | 2.65 | 8.16 | 0.29 | 13.91 | 0.034 | 2.01 | 1.44 |
| Legend | Nov. 23, 2010 | 1.30 | 8.51 | 0.24 | 12.99 | 0.031 | 2.07 | 4.86 |
| Zapotec | Nov. 23, 2010 | 2.06 | 5.49 | 0.24 | 11.78 | 0.036 | 2.50 | 1.62 |
| Sunray | Dec. 1, 2010 | 3.94 | 18.52 | 0.40 | 20.03 | 0.020 | 0.70 | 9.78 |
| Martian Giant | Dec. 1, 2010 | 4.86 | 19.00 | 0.26 | 16.25 | 0.027 | 0.91 | 9.77 |
| Rose Tomato | Dec. 1, 2010 | 1.48 | 6.31 | 0.35 | 8.40 | 0.034 | 0.52 | 1.28 |
| Mr. Stripey | Dec. 1, 2010 | 3.18 | 10.67 | 0.17 | 12.88 | 0.014 | 1.19 | 6.60 |
| Tasti-Lee | Dec. 1, 2010 | 3.10 | 11.53 | 0.14 | 11.44 | 0.011 | 0.54 | 7.02 |
| Costoluto Genovese | Dec. 1, 2010 | 4.84 | 11.40 | 0.23 | 10.69 | 0.042 | 1.18 | 3.62 |

TABLE 8P

| | | 2- | | | | 2-methyl- | | |
|---|---|---|---|---|---|---|---|---|
| | | methylbutyl | 2- | 2-methyl- | 2- | 2- | 2- | butyl |
| | panel | acetate | butylacetate | 1-butanol | methylbutanal | butenal | isobutylthiazole | acetate |
| Variety | date | ng/gfw/hr | ng/gfw/hr | ng/gfw/hr | ng/gfw/hr | ng/gfw/hr | ng/gfw/hr | ng/gfw/hr |
| Cherry Roma | Feb. 24, 2010 | 0.15 | 0.01 | 21.28 | 5.45 | 14.42 | 17.42 | 0.00 |
| Ailsa Craig | Feb. 24, 2010 | 0.89 | 0.04 | 27.09 | 3.99 | 9.89 | 5.87 | 0.13 |
| Red Pear | Feb. 24, 2010 | 0.14 | 0.01 | 7.28 | 2.70 | 4.41 | 4.62 | 0.05 |
| Tommy Toe | Feb. 24, 2010 | 0.70 | 0.06 | 18.30 | 3.34 | 6.53 | 3.70 | 0.08 |
| Supermarket Cherry | Feb. 24, 2010 | 0.04 | 0.02 | 6.84 | 2.14 | 5.28 | 1.09 | 0.00 |
| Porter | Feb. 24, 2010 | 0.09 | 0.01 | 2.92 | 1.38 | 2.36 | 1.73 | 0.05 |
| Matina | Mar. 2, 2010 | 1.13 | 0.01 | 23.30 | 2.66 | 6.39 | 9.27 | 0.13 |
| Bloody Butcher | Mar. 2, 2010 | 0.73 | 0.02 | 6.46 | 2.64 | 2.13 | 4.83 | 0.16 |
| Chadwick Cherry | Mar. 2, 2010 | 0.71 | 0.00 | 8.78 | 1.99 | 3.14 | 10.48 | 0.09 |
| Thai Pink Cherry | Mar. 2, 2010 | 0.19 | 0.03 | 5.92 | 2.38 | 3.10 | 5.67 | 0.05 |
| Peacevine Cherry | Mar. 2, 2010 | 0.73 | 0.06 | 40.81 | 6.55 | 9.34 | 14.16 | 0.07 |
| Supermarket Cherry | Mar. 2, 2010 | 0.05 | 0.01 | 31.49 | 6.35 | 19.03 | 0.80 | 0.01 |
| Stupice | Mar. 2, 2010 | 0.74 | 0.04 | 16.51 | 3.06 | 7.10 | 7.63 | 0.10 |
| Large Red Cherry | Mar. 2, 2010 | 1.10 | 0.01 | 38.76 | 5.18 | 8.55 | 5.73 | 0.06 |
| Bloody Butcher | Mar. 18, 2010 | 0.43 | 0.01 | 5.43 | 2.15 | 2.48 | 8.28 | 0.10 |
| Giant Belgium | Mar. 18, 2010 | 0.52 | 0.00 | 18.14 | 3.43 | 7.78 | 5.63 | 0.11 |
| Thessaloniki | Mar. 18, 2010 | 0.72 | 0.03 | 15.11 | 3.50 | 5.91 | 9.64 | 0.09 |
| Supermarket Round | Mar. 18, 2010 | 4.10 | 0.06 | 33.35 | 4.51 | 18.99 | 2.75 | 0.12 |
| Marmande VFA | Mar. 18, 2010 | 0.14 | 0.01 | 5.18 | 1.15 | 3.56 | 4.52 | 0.04 |
| Red Calabash | Apr. 1, 2010 | 1.04 | 0.06 | 21.39 | 2.40 | 10.23 | 6.61 | 0.19 |
| Brandywine | Apr. 1, 2010 | 0.19 | 0.04 | 3.75 | 1.18 | 4.30 | 2.73 | 0.15 |
| Dixie Golden Giant | Apr. 1, 2010 | 0.94 | 0.06 | 3.37 | 1.90 | 6.72 | 1.93 | 0.68 |
| Aunt Ruby's German Green | Apr. 1, 2010 | 0.96 | 0.01 | 28.44 | 3.85 | 13.41 | 8.02 | 0.13 |
| Thessaloniki | Apr. 1, 2010 | 0.14 | 0.00 | 12.31 | 1.74 | 3.91 | 6.67 | 0.06 |
| Supermarket Round | Apr. 1, 2010 | 0.31 | 0.53 | 5.74 | 1.23 | 4.93 | 1.03 | 0.06 |

TABLE 8Q

| | | | | 2- | | 2- | | |
|---|---|---|---|---|---|---|---|---|
| | | 2- | | methyl- | | methyl- | | |
| | | methylbutyl | 2- | 1- | 2- | 2- | 2- | butyl |
| | panel | acetate | butylacetate | butanol | methylbutanal | butenal | isobutylthiazole | acetate |
| Variety | date | ng/gfw/hr | ng/gfw/hr | ng/gfw/hr | ng/gfw/hr | ng/gfw/hr | ng/gfw/hr | ng/gfw/hr |
| Supermarket Round | Jun. 11, 2010 | 2.12 | 0.04 | 15.59 | 3.25 | 7.52 | 3.99 | 0.20 |
| Three Sisters | Jun. 11, 2010 | 2.54 | 0.02 | 23.14 | 5.17 | 20.56 | 9.02 | 0.20 |
| Skorospelka Red | Jun. 11, 2010 | 1.91 | 0.66 | 21.33 | 3.47 | 14.50 | 7.92 | 0.21 |
| Yellow Perfection | Jun. 11, 2010 | 2.98 | 0.49 | 24.86 | 5.20 | 13.23 | 6.18 | 0.24 |
| Clear Pink Slicer | Jun. 11, 2010 | 7.14 | 0.36 | 42.81 | 4.30 | 19.79 | 6.99 | 0.32 |
| Tigerella Orange | Jun. 11, 2010 | 1.52 | 0.13 | 9.53 | 3.08 | 7.99 | 1.35 | 0.24 |
| Gulf State Market | Jun. 17, 2010 | 3.38 | 0.86 | 34.40 | 2.62 | 15.62 | 7.18 | 0.16 |
| Bloody Butcher | Jun. 17, 2010 | 0.69 | 0.09 | 3.76 | 2.09 | 2.00 | 3.39 | 0.16 |
| Garden Peach | Jun. 17, 2010 | 1.40 | 0.23 | 16.53 | 3.45 | 5.94 | 2.14 | 0.11 |
| Amish Salad | Jun. 17, 2010 | 0.27 | 0.16 | 2.66 | 3.09 | 2.97 | 2.45 | 0.14 |
| Ailsa Craig | Jun. 17, 2010 | 0.86 | 0.06 | 5.69 | 2.49 | 3.97 | 2.74 | 0.21 |
| Supermarket Round | Jun. 17, 2010 | 6.86 | 0.08 | 28.00 | 5.15 | 15.79 | 4.67 | 0.41 |
| Super Sioux | Jun. 30, 2010 | 1.81 | 0.42 | 22.80 | 4.51 | 8.36 | 5.61 | 0.17 |
| St. Pierre | Jun. 30, 2010 | 1.35 | 0.77 | 9.57 | 3.33 | 3.50 | 5.60 | 0.12 |
| Supermarket Round | Jun. 30, 2010 | 0.78 | 0.02 | 6.38 | 4.40 | 6.15 | 2.89 | 0.09 |
| Kentucky Beefsteak | Jun. 30, 2010 | 0.24 | 0.29 | 2.12 | 3.06 | 1.45 | 0.89 | 0.04 |
| Green Zebra | Jun. 30, 2010 | 0.95 | 0.67 | 14.42 | 3.96 | 5.18 | 9.40 | 0.17 |
| Zapotec | Jun. 30, 2010 | 2.11 | 0.27 | 19.88 | 5.66 | 12.32 | 2.58 | 0.11 |
| Supermarket Cherry | Jul. 8, 2010 | 0.11 | 0.00 | 21.15 | 6.02 | 11.60 | 0.74 | 0.01 |
| Yellow Jelly Bean | Jul. 8, 2010 | 0.13 | 0.02 | 8.39 | 3.89 | 4.53 | 17.53 | 0.01 |
| Mexico Midget | Jul. 8, 2010 | 0.28 | 0.01 | 24.66 | 6.39 | 8.86 | 11.82 | 0.02 |
| Lemon Drop | Jul. 8, 2010 | 0.32 | 0.02 | 5.54 | 4.05 | 5.97 | 13.91 | 0.03 |
| LA1482 | Jul. 8, 2010 | 1.11 | 0.02 | 7.33 | 4.56 | 5.52 | 7.32 | 0.13 |
| Matt's Wild Cherry | Jul. 8, 2010 | 0.18 | 0.01 | 8.90 | 3.18 | 1.95 | 13.77 | 0.04 |

TABLE 8R

| | | 2-methylbutyl | 2- | 2-methyl- | 2- | 2-methyl- | 2- | |
|---|---|---|---|---|---|---|---|---|
| | | acetate | butylacetate | 1-butanol | methylbutanal | 2-butenal | isobutylthiazole | butyl acetate |
| Variety | panel date | ng/gfw/hr | ng/gfw/hr | ng/gfw/hr | ng/gfw/hr | ng/gfw/hr | ng/gfw/hr | ng/gfw/hr |
| Maglia Rosa Cherry | Nov. 3, 2010 | 1.28 | 0.30 | 17.13 | 4.95 | 5.59 | 1.48 | 0.26 |
| Wisconsin 55 | Nov. 3, 2010 | 4.00 | 0.18 | 42.95 | 5.99 | 21.77 | 4.56 | 0.52 |
| Glacier | Nov. 3, 2010 | 1.65 | 0.19 | 7.11 | 4.36 | 10.65 | 1.53 | 0.48 |
| Oregon Spring Bush | Nov. 3, 2010 | 1.42 | 1.01 | 8.87 | 3.73 | 12.31 | 2.74 | 0.30 |
| Santiam | Nov. 3, 2010 | 1.96 | 0.86 | 8.99 | 3.98 | 8.47 | 1.32 | 0.59 |
| Giant Oxheart | Nov. 3, 2010 | 1.17 | 0.51 | 5.56 | 4.16 | 10.61 | 2.76 | 0.39 |
| Micado Violettor | Nov. 12, 2010 | 1.16 | 0.16 | 12.31 | 8.20 | 21.40 | 0.79 | 0.20 |
| Livingston's Globe | Nov. 12, 2010 | 0.74 | 0.15 | 7.07 | 4.36 | 7.23 | 0.92 | 0.14 |
| Old Brooks | Nov. 12, 2010 | 0.49 | 0.31 | 5.27 | 4.28 | 4.31 | 5.68 | 0.12 |
| Park's Whopper | Nov. 12, 2010 | 0.95 | 0.58 | 5.68 | 4.25 | 5.45 | 2.81 | 0.40 |
| Hillbilly | Nov. 12, 2010 | 0.64 | 0.07 | 8.94 | 4.96 | 6.33 | 2.15 | 0.17 |
| German Head | Nov. 12, 2010 | 0.77 | 0.04 | 10.67 | 6.27 | 11.69 | 2.41 | 0.16 |
| Livingston's Stone | Nov. 17, 2010 | 0.53 | 0.05 | 3.57 | 3.63 | 2.97 | 1.40 | 0.14 |
| Ponderosa Pink | Nov. 17, 2010 | 1.39 | 0.02 | 16.67 | 8.18 | 19.59 | 1.69 | 0.28 |
| Cherokee Purple | Nov. 17, 2010 | 0.80 | 0.29 | 5.82 | 5.43 | 7.48 | 2.75 | 0.27 |
| Black | Nov. 17, 2010 | 0.24 | 0.06 | 2.59 | 2.47 | 3.22 | 2.37 | 0.10 |
| Nyagous | Nov. 17, 2010 | 0.73 | 0.06 | 3.82 | 3.65 | 4.56 | 1.10 | 0.15 |
| Oaxacan Pink | Nov. 17, 2010 | 0.81 | 0.07 | 15.55 | 6.06 | 12.35 | 3.67 | 0.13 |
| Livingston's Golden Queen | Nov. 23, 2010 | 1.04 | 0.06 | 5.76 | 5.59 | 9.02 | 1.56 | 0.36 |
| Ponderosa Red | Nov. 23, 2010 | 2.31 | 0.27 | 8.12 | 4.47 | 6.24 | 2.28 | 0.35 |
| Japanese Black Trifele | Nov. 23, 2010 | 0.90 | 0.07 | 3.24 | 3.11 | 3.67 | 0.52 | 0.16 |
| Crimson Sprinter | Nov. 23, 2010 | 2.62 | 1.08 | 11.14 | 5.02 | 7.17 | 2.19 | 0.50 |
| Legend | Nov. 23, 2010 | 2.81 | 0.15 | 13.95 | 7.21 | 14.94 | 1.41 | 0.43 |
| Zapotec | Nov. 23, 2010 | 2.17 | 0.05 | 25.67 | 7.33 | 22.56 | 1.60 | 0.19 |
| Sunray | Dec. 1, 2010 | 1.11 | 0.21 | 6.70 | 7.40 | 10.24 | 2.22 | 0.46 |
| Martian Giant | Dec. 1, 2010 | 1.43 | 0.07 | 6.17 | 6.54 | 6.25 | 1.94 | 0.46 |
| Rose Tomato | Dec. 1, 2010 | 0.49 | 0.22 | 2.80 | 4.25 | 4.78 | 1.49 | 0.34 |
| Mr. Stripey | Dec. 1, 2010 | 1.07 | 0.06 | 8.42 | 8.51 | 7.48 | 1.84 | 0.24 |
| Tasti-Lee | Dec. 1, 2010 | 0.56 | 0.08 | 2.62 | 4.81 | 4.91 | 1.29 | 0.19 |
| Costoluto Genovese | Dec. 1, 2010 | 2.06 | 0.10 | 9.78 | 7.60 | 11.15 | 4.28 | 0.55 |

TABLE 8S

| | | | 6-methyl-5- | | | | | | |
|---|---|---|---|---|---|---|---|---|---|
| | | 6-methyl-5- | hepten-2- | β- | | | | | β- |
| | panel | hepten-2-ol | one | cyclocitral | geranial | β-ionone | geranylacetone | neral | damascenone |
| Variety | date | ng/gfw/hr | ng/gfw/hr | ng/gfw/hr | ng/gfw/hr | ng/gfw/hr | ng/gfw/hr | ng/gfw/hr | ng/gfw/hr |
| Cherry Roma | Feb. 24, 2010 | 0.390 | 8.53 | 0.111 | 0.231 | 0.096 | 4.41 | 0.297 | 0.0072 |
| Ailsa Craig | Feb. 24, 2010 | 0.166 | 2.91 | 0.199 | 0.114 | 0.143 | 1.24 | 0.239 | 0.0030 |
| Red Pear | Feb. 24, 2010 | 0.138 | 4.84 | 0.141 | 0.147 | 0.053 | 1.26 | 0.157 | 0.0032 |
| Tommy Toe | Feb. 24, 2010 | 0.151 | 4.00 | 0.151 | 0.150 | 0.094 | 1.32 | 0.177 | 0.0033 |
| Supermarket Cherry | Feb. 24, 2010 | 0.087 | 4.23 | 0.081 | 0.075 | 0.025 | 1.58 | 0.093 | 0.0026 |
| Porter | Feb. 24, 2010 | 0.238 | 6.30 | 0.145 | 0.224 | 0.059 | 1.82 | 0.143 | 0.0033 |
| Matina | Mar. 2, 2010 | 0.299 | 5.47 | 0.143 | 0.250 | 0.114 | 2.43 | 0.202 | 0.0054 |
| Bloody Butcher | Mar. 2, 2010 | 0.154 | 4.58 | 0.121 | 0.161 | 0.045 | 1.17 | 0.166 | 0.0030 |
| Chadwick Cherry | Mar. 2, 2010 | 0.296 | 7.27 | 0.199 | 0.339 | 0.107 | 1.97 | 0.185 | 0.0019 |
| Thai Pink Cherry | Mar. 2, 2010 | 0.188 | 8.77 | 0.149 | 0.329 | 0.065 | 2.60 | 0.202 | 0.0020 |
| Peacevine Cherry | Mar. 2, 2010 | 0.489 | 9.25 | 0.213 | 0.293 | 0.106 | 3.45 | 0.201 | 0.0049 |
| Supermarket Cherry | Mar. 2, 2010 | 0.075 | 2.30 | 0.063 | 0.043 | 0.024 | 0.71 | 0.065 | 0.0042 |
| Stupice | Mar. 2, 2010 | 0.358 | 7.26 | 0.176 | 0.291 | 0.119 | 2.61 | 0.183 | 0.0016 |
| Large Red Cherry | Mar. 2, 2010 | 0.194 | 8.20 | 0.230 | 0.323 | 0.179 | 2.96 | 0.222 | 0.0030 |
| Bloody Butcher | Mar. 18, 2010 | 0.216 | 5.37 | 0.145 | 0.194 | 0.068 | 1.53 | 0.119 | 0.0094 |
| Giant Belgium | Mar. 18, 2010 | 0.323 | 8.05 | 0.125 | 0.298 | 0.071 | 2.52 | 0.216 | 0.0091 |
| Thessaloniki | Mar. 18, 2010 | 0.129 | 5.35 | 0.131 | 0.175 | 0.041 | 1.31 | 0.164 | 0.0040 |
| Supermarket Round | Mar. 18, 2010 | 0.062 | 5.59 | 0.083 | 0.090 | 0.028 | 0.78 | 0.155 | 0.0061 |
| Marmande VFA | Mar. 18, 2010 | 0.121 | 3.94 | 0.093 | 0.190 | 0.035 | 1.10 | 0.308 | 0.0070 |
| Red Calabash | Apr. 1, 2010 | 0.090 | 3.18 | 0.082 | 0.119 | 0.046 | 1.17 | 0.145 | 0.0021 |
| Brandywine | Apr. 1, 2010 | 0.209 | 3.55 | 0.056 | 0.161 | 0.038 | 1.33 | 0.108 | 0.0023 |
| Dixie Golden Giant | Apr. 1, 2010 | 0.116 | 3.94 | 0.027 | 0.075 | 0.028 | 3.50 | 0.149 | 0.0092 |
| Aunt Ruby's German | Apr. 1, 2010 | 0.090 | 0.28 | 0.034 | 0.015 | 0.031 | 0.06 | 0.192 | 0.0025 |
| Green | | | | | | | | | |
| Thessaloniki | Apr. 1, 2010 | 0.064 | 3.06 | 0.101 | 0.119 | 0.052 | 0.69 | 0.109 | 0.0062 |
| Supermarket Round | Apr. 1, 2010 | 0.065 | 3.93 | 0.074 | 0.078 | 0.030 | 1.14 | 0.108 | 0.0042 |

TABLE 8T

| | | 6- | 6- | | | | | | |
|---|---|---|---|---|---|---|---|---|---|
| | | methyl- | methyl- | | | | | | |
| | | 5- | 5- | | | | | | |
| | | hepten- | hepten- | β- | | | | | β- |
| | panel | 2-ol | 2-one | cyclocitral | geranial | β-ionone | geranylacetone | neral | damascenone |
| Variety | date | ng/gfw/hr | ng/gfw/hr | ng/gfw/hr | ng/gfw/hr | ng/gfw/hr | ng/gfw/hr | ng/gfw/hr | ng/gfw/hr |
| Supermarket Round | Jun. 11, 2010 | 0.263 | 9.05 | 0.050 | 0.209 | 0.019 | 3.04 | 0.270 | 0.0043 |
| Three Sisters | Jun. 11, 2010 | 0.279 | 5.29 | 0.096 | 0.230 | 0.063 | 2.04 | 0.186 | 0.0033 |
| Skorospelka Red | Jun. 11, 2010 | 0.217 | 5.27 | 0.061 | 0.247 | 0.032 | 2.49 | 0.202 | 0.0027 |
| Yellow Perfection | Jun. 11, 2010 | 0.103 | 0.29 | 0.024 | 0.011 | 0.015 | 0.05 | 0.194 | 0.0065 |
| Clear Pink Slicer | Jun. 11, 2010 | 0.154 | 3.01 | 0.052 | 0.118 | 0.034 | 1.16 | 0.191 | 0.0053 |
| Tigerella Orange | Jun. 11, 2010 | 0.128 | 1.68 | 0.079 | 0.083 | 0.050 | 0.76 | 0.133 | 0.0030 |
| Gulf State Market | Jun. 17, 2010 | 0.266 | 4.57 | 0.055 | 0.169 | 0.024 | 2.35 | 0.187 | 0.0010 |
| Bloody Butcher | Jun. 17, 2010 | 0.139 | 2.72 | 0.078 | 0.137 | 0.047 | 1.78 | 0.186 | 0.0009 |
| Garden Peach | Jun. 17, 2010 | 0.029 | 0.23 | 0.012 | 0.005 | 0.006 | 0.03 | 0.126 | 0.0019 |
| Amish Salad | Jun. 17, 2010 | 0.147 | 2.47 | 0.049 | 0.106 | 0.015 | 0.90 | 0.179 | 0.0021 |
| Ailsa Craig | Jun. 17, 2010 | 0.160 | 1.74 | 0.094 | 0.106 | 0.072 | 0.89 | 0.211 | 0.0010 |
| Supermarket Round | Jun. 17, 2010 | 0.221 | 7.73 | 0.075 | 0.114 | 0.030 | 2.63 | 0.215 | 0.0022 |
| Super Sioux | Jun. 30, 2010 | 0.143 | 2.59 | 0.043 | 0.084 | 0.019 | 0.86 | 0.153 | 0.0022 |
| St. Pierre | Jun. 30, 2010 | 0.264 | 3.00 | 0.061 | 0.155 | 0.036 | 1.11 | 0.110 | 0.0022 |
| Supermarket Round | Jun. 30, 2010 | 0.108 | 4.29 | 0.034 | 0.094 | 0.011 | 1.32 | 0.096 | 0.0021 |
| Kentucky Beefsteak | Jun. 30, 2010 | 0.129 | 5.69 | 0.025 | 0.094 | 0.009 | 9.58 | 0.107 | 0.0039 |
| Green Zebra | Jun. 30, 2010 | 0.074 | 0.20 | 0.015 | 0.013 | 0.009 | 0.03 | 0.106 | 0.0020 |
| Zapotec | Jun. 30, 2010 | 0.111 | 2.00 | 0.033 | 0.070 | 0.009 | 0.46 | 0.117 | 0.0025 |
| Supermarket Cherry | Jul. 8, 2010 | 0.145 | 4.48 | 0.032 | 0.070 | 0.016 | 0.94 | 0.111 | 0.0010 |
| Yellow Jelly Bean | Jul. 8, 2010 | 0.054 | 0.12 | 0.016 | 0.007 | 0.011 | 0.02 | 0.090 | 0.0021 |
| Mexico Midget | Jul. 8, 2010 | 0.105 | 2.55 | 0.044 | 0.084 | 0.037 | 0.67 | 0.113 | 0.0018 |
| Lemon Drop | Jul. 8, 2010 | 0.087 | 0.13 | 0.015 | 0.008 | 0.010 | 0.02 | 0.069 | 0.0017 |
| LA1482 | Jul. 8, 2010 | 0.154 | 2.26 | 0.058 | 0.144 | 0.023 | 0.69 | 0.160 | 0.0019 |
| Matt's Wild Cherry | Jul. 8, 2010 | 0.077 | 1.48 | 0.030 | 0.050 | 0.032 | 0.39 | 0.161 | 0.0032 |

TABLE 8U

| | | 6-methyl-5- | 6-methyl-5- | | | | | | |
|---|---|---|---|---|---|---|---|---|---|
| | | hepten-2-ol | hepten-2-one | β-cyclocitral | geranial | β-ionone | geranylacetone | neral | β-damascenone |
| Variety | panel date | ng/gfw/hr | ng/gfw/hr | ng/gfw/hr | ng/gfw/hr | ng/gfw/hr | ng/gfw/hr | ng/gfw/hr | ng/gfw/hr |
| Maglia Rosa Cherry | Nov. 3, 2010 | 0.143 | 1.51 | 0.085 | 0.061 | 0.048 | 0.49 | 0.165 | 0.0629 |
| Wisconsin 55 | Nov. 3, 2010 | 0.110 | 4.54 | 0.082 | 0.201 | 0.049 | 1.90 | 0.270 | 0.0636 |
| Glacier | Nov. 3, 2010 | 0.097 | 1.56 | 0.060 | 0.073 | 0.026 | 0.62 | 0.130 | 0.0248 |
| Oregon Spring Bush | Nov. 3, 2010 | 0.299 | 4.08 | 0.045 | 0.157 | 0.016 | 1.43 | 0.166 | 0.0113 |
| Santiam | Nov. 3, 2010 | 0.105 | 2.33 | 0.055 | 0.093 | 0.021 | 0.86 | 0.168 | 0.0163 |
| Giant Oxheart | Nov. 3, 2010 | 0.166 | 3.39 | 0.084 | 0.141 | 0.055 | 1.32 | 0.169 | 0.0605 |
| Micado Violettor | Nov. 12, 2010 | 0.010 | 1.99 | 0.054 | 0.062 | 0.026 | 0.57 | 0.137 | 0.0018 |
| Livingston's Globe | Nov. 12, 2010 | 0.012 | 2.72 | 0.049 | 0.072 | 0.018 | 0.62 | 0.190 | 0.0028 |
| Old Brooks | Nov. 12, 2010 | 0.032 | 4.87 | 0.086 | 0.195 | 0.045 | 1.52 | 0.152 | 0.0033 |
| Park's Whopper | Nov. 12, 2010 | 0.052 | 3.45 | 0.094 | 0.105 | 0.030 | 1.14 | 0.093 | 0.0018 |
| Hillbilly | Nov. 12, 2010 | 0.025 | 0.25 | 0.093 | 0.016 | 0.064 | 0.04 | 0.099 | 0.0022 |
| German Head | Nov. 12, 2010 | 0.033 | 3.39 | 0.056 | 0.090 | 0.022 | 0.91 | 0.109 | 0.0023 |
| Livingston's Stone | Nov. 17, 2010 | 0.066 | 3.10 | 0.058 | 0.121 | 0.017 | 0.86 | 0.085 | 0.0104 |
| Ponderosa Pink | Nov. 17, 2010 | 0.154 | 5.63 | 0.069 | 0.145 | 0.019 | 1.30 | 0.119 | 0.0186 |
| Cherokee Purple | Nov. 17, 2010 | 0.110 | 4.13 | 0.064 | 0.137 | 0.019 | 0.91 | 0.141 | 0.0152 |
| Black | Nov. 17, 2010 | 0.041 | 2.46 | 0.045 | 0.083 | 0.032 | 0.42 | 0.093 | 0.0070 |
| Nyagous | Nov. 17, 2010 | 0.042 | 2.34 | 0.048 | 0.104 | 0.028 | 0.31 | 0.134 | 0.0067 |
| Oaxacan Pink | Nov. 17, 2010 | 0.021 | 1.74 | 0.069 | 0.072 | 0.061 | 0.67 | 0.075 | 0.1035 |
| Livingston's Golden | Nov. 23, 2010 | 0.018 | 0.23 | 0.020 | 0.010 | 0.014 | 0.03 | 0.059 | 0.0025 |
| Queen | | | | | | | | | |
| Ponderosa Red | Nov. 23, 2010 | 0.044 | 2.43 | 0.075 | 0.076 | 0.021 | 0.70 | 0.082 | 0.0042 |
| Japanese Black | Nov. 23, 2010 | 0.053 | 1.44 | 0.036 | 0.057 | 0.021 | 0.22 | 0.050 | 0.0033 |
| Trifele | | | | | | | | | |
| Crimson Sprinter | Nov. 23, 2010 | 0.038 | 2.58 | 0.038 | 0.103 | 0.016 | 1.27 | 0.099 | 0.0044 |
| Legend | Nov. 23, 2010 | 0.028 | 2.79 | 0.064 | 0.073 | 0.024 | 0.81 | 0.100 | 0.0044 |
| Zapotec | Nov. 23, 2010 | 0.078 | 2.52 | 0.069 | 0.093 | 0.022 | 0.52 | 0.083 | 0.0027 |
| Sunray | Dec. 1, 2010 | 0.021 | 3.59 | 0.026 | 0.045 | 0.009 | 2.59 | 0.063 | 0.0036 |
| Martian Giant | Dec. 1, 2010 | 0.035 | 3.66 | 0.070 | 0.088 | 0.016 | 0.78 | 0.070 | 0.0031 |
| Rose Tomato | Dec. 1, 2010 | 0.010 | 3.26 | 0.066 | 0.125 | 0.030 | 1.04 | 0.080 | 0.0014 |
| Mr. Stripey | Dec. 1, 2010 | 0.029 | 0.23 | 0.048 | 0.007 | 0.023 | 0.03 | 0.040 | 0.0024 |
| Tasti-Lee | Dec. 1, 2010 | 0.012 | 3.17 | 0.032 | 0.067 | 0.011 | 0.72 | 0.058 | 0.0016 |
| Costoluto Genovese | Dec. 1, 2010 | 0.029 | 3.53 | 0.080 | 0.123 | 0.024 | 0.89 | 0.081 | 0.0016 |

TABLE 8V

| | | benzyl | | | | 2-phenyl |
|---|---|---|---|---|---|---|
| | panel | cyanide | phenylacetaldehyde | benzaldehyde | eugenol | ethanol |
| Variety | date | ng/gfw/hr | ng/gfw/hr | ng/gfw/hr | ng/gfw/hr | ng/gfw/hr |
| Cherry Roma | Feb. 24, 2010 | 3.318 | 2.765 | 12.33 | 0.001 | 7.917 |
| Ailsa Craig | Feb. 24, 2010 | 0.046 | 0.078 | 4.18 | 1.273 | 0.038 |
| Red Pear | Feb. 24, 2010 | 0.121 | 0.141 | 5.15 | 0.342 | 0.033 |
| Tommy Toe | Feb. 24, 2010 | 0.137 | 0.254 | 7.72 | 0.312 | 0.126 |
| Supermarket Cherry | Feb. 24, 2010 | 0.723 | 0.436 | 1.97 | 0.009 | 1.093 |
| Porter | Feb. 24, 2010 | 0.196 | 0.072 | 1.73 | 0.008 | 0.108 |
| Matina | Mar. 2, 2010 | 0.049 | 0.043 | 3.66 | 0.308 | 0.034 |
| Bloody Butcher | Mar. 2, 2010 | 0.034 | 0.051 | 1.35 | 0.180 | 0.026 |
| Chadwick Cherry | Mar. 2, 2010 | 0.565 | 0.139 | 3.18 | 0.461 | 0.042 |
| Thai Pink Cherry | Mar. 2, 2010 | 0.038 | 0.098 | 1.00 | 0.018 | 0.031 |
| Peacevine Cherry | Mar. 2, 2010 | 1.939 | 0.461 | 6.87 | 0.024 | 1.855 |
| Supermarket Cherry | Mar. 2, 2010 | 0.043 | 0.277 | 2.37 | 0.006 | 0.568 |
| Stupice | Mar. 2, 2010 | 0.074 | 0.205 | 3.93 | 0.178 | 0.020 |
| Large Red Cherry | Mar. 2, 2010 | 0.202 | 0.448 | 13.39 | 0.500 | 0.036 |
| Bloody Butcher | Mar. 18, 2010 | 0.042 | 0.022 | 1.35 | 0.093 | 0.033 |
| Giant Belgium | Mar. 18, 2010 | 0.058 | 0.182 | 1.90 | 0.010 | 0.018 |
| Thessaloniki | Mar. 18, 2010 | 0.059 | 0.090 | 4.15 | 0.840 | 0.022 |
| Supermarket Round | Mar. 18, 2010 | 0.073 | 0.565 | 2.11 | 0.246 | 1.464 |
| Marmande VFA | Mar. 18, 2010 | 0.027 | 0.010 | 2.00 | 0.014 | 0.028 |
| Red Calabash | Apr. 1, 2010 | 0.044 | 0.070 | 1.43 | 0.474 | 0.024 |
| Brandywine | Apr. 1, 2010 | 0.069 | 0.222 | 0.63 | 0.209 | 0.060 |
| Dixie Golden Giant | Apr. 1, 2010 | 0.044 | 0.073 | 0.61 | 0.009 | 0.006 |
| Aunt Ruby's German | Apr. 1, 2010 | 0.266 | 0.241 | 0.89 | 0.277 | 0.061 |
| Green | | | | | | |
| Thessaloniki | Apr. 1, 2010 | 0.030 | 0.033 | 2.16 | 0.799 | 0.039 |
| Supermarket Round | Apr. 1, 2010 | 0.054 | 0.197 | 0.71 | 0.195 | 0.818 |

| | | 1-nitro-2- | benzyl | | |
|---|---|---|---|---|---|
| | salicylaldehyde | phenylethane | alcohol | methylsalicylate | guaiacol |
| Variety | ng/gfw/hr | ng/gfw/hr | ng/gfw/hr | ng/gfw/hr | ng/gfw/hr |
| Cherry Roma | 0.02 | 3.137 | 0.61 | 0.011 | 0.068 |
| Ailsa Craig | 0.41 | 0.082 | 0.39 | 0.281 | 0.649 |
| Red Pear | 0.84 | 0.366 | 0.33 | 0.695 | 1.449 |
| Tommy Toe | 0.41 | 0.326 | 0.36 | 1.461 | 0.604 |
| Supermarket Cherry | 0.06 | 0.343 | 0.05 | 0.011 | 0.033 |
| Porter | 0.03 | 0.349 | 0.10 | 0.008 | 0.060 |
| Matina | 0.77 | 0.145 | 0.19 | 0.287 | 0.972 |
| Bloody Butcher | 0.55 | 0.182 | 0.22 | 0.178 | 1.272 |
| Chadwick Cherry | 0.36 | 1.074 | 0.50 | 1.178 | 0.866 |
| Thai Pink Cherry | 0.05 | 0.347 | 0.13 | 0.041 | 0.089 |
| Peacevine Cherry | 0.04 | 4.164 | 0.60 | 0.012 | 0.075 |
| Supermarket Cherry | 0.17 | 0.199 | 0.03 | 0.027 | 0.036 |
| Stupice | 0.74 | 0.563 | 0.32 | 0.281 | 1.107 |
| Large Red Cherry | 0.59 | 0.908 | 0.37 | 1.710 | 0.895 |
| Bloody Butcher | 0.28 | 0.294 | 0.16 | 0.137 | 0.703 |
| Giant Belgium | 0.12 | 0.827 | 0.19 | 0.020 | 0.205 |
| Thessaloniki | 0.87 | 0.569 | 0.34 | 0.360 | 2.005 |
| Supermarket Round | 0.12 | 1.036 | 0.15 | 0.209 | 0.182 |
| Marmande VFA | 0.39 | 0.089 | 0.53 | 0.016 | 0.175 |
| Red Calabash | 0.31 | 0.101 | 0.24 | 1.016 | 1.278 |
| Brandywine | 0.60 | 0.147 | 0.26 | 2.277 | 4.936 |
| Dixie Golden Giant | 0.05 | 0.062 | 0.31 | 0.088 | 0.092 |
| Aunt Ruby's German | 0.42 | 1.665 | 0.54 | 2.059 | 3.747 |
| Green | | | | | |
| Thessaloniki | 0.41 | 0.259 | 0.21 | 0.239 | 1.462 |
| Supermarket Round | 0.11 | 0.332 | 0.04 | 0.148 | 0.093 |

TABLE 8W

| | | benzyl | | | | 2-phenyl |
|---|---|---|---|---|---|---|
| | panel | cyanide | phenylacetaldehyde | benzaldehyde | eugenol | ethanol |
| Variety | date | ng/gfw/hr | ng/gfw/hr | ng/gfw/hr | ng/gfw/hr | ng/gfw/hr |
| Supermarket Round | Jun. 11. 2010 | 0.325 | 0.449 | 3.33 | 0.418 | 2.029 |
| Three Sisters | Jun. 11. 2010 | 0.282 | 0.237 | 4.10 | 0.354 | 0.665 |
| Skorospelka Red | Jun. 11. 2010 | 0.082 | 0.130 | 2.00 | 0.017 | 0.113 |
| Yellow Perfection | Jun. 11. 2010 | 0.034 | 0.115 | 3.61 | 1.959 | 0.103 |
| Clear Pink Slicer | Jun. 11. 2010 | 0.113 | 0.495 | 4.85 | 1.648 | 0.744 |
| Tigerella Orange | Jun. 11. 2010 | 0.010 | 0.136 | 0.54 | 1.733 | 0.006 |
| Gulf State Market | Jun. 17, 2010 | 0.213 | 1.059 | 7.35 | 0.949 | 4.541 |
| Bloody Butcher | Jun. 17, 2010 | 0.079 | 0.060 | 1.73 | 0.270 | 0.035 |
| Garden Peach | Jun. 17, 2010 | 0.074 | 0.173 | 3.56 | 0.749 | 0.207 |
| Amish Salad | Jun. 17, 2010 | 0.108 | 0.125 | 3.79 | 0.006 | 0.185 |
| Ailsa Craig | Jun. 17, 2010 | 0.038 | 0.110 | 0.73 | 0.939 | 0.032 |
| Supermarket Round | Jun. 17, 2010 | 0.170 | 0.239 | 2.29 | 0.285 | 1.453 |
| Super Sioux | Jun. 30, 2010 | 0.158 | 0.208 | 2.56 | 0.102 | 0.184 |
| St. Pierre | Jun. 30, 2010 | 0.299 | 0.013 | 2.16 | 0.405 | 0.003 |
| Supermarket Round | Jun. 30, 2010 | 0.161 | 0.195 | 2.10 | 0.149 | 0.382 |
| Kentucky Beefsteak | Jun. 30, 2010 | 0.105 | 0.030 | 1.32 | 0.003 | 0.084 |
| Green Zebra | Jun. 30, 2010 | 0.128 | 0.173 | 7.12 | 0.572 | 0.051 |
| Zapotec | Jun. 30, 2010 | 0.163 | 0.188 | 3.14 | 0.358 | 0.381 |
| Supermarket Cherry | Jul. 8, 2010 | 1.214 | 1.211 | 8.67 | 0.001 | 4.568 |
| Yellow Jelly Bean | Jul. 8, 2010 | 0.728 | 0.201 | 1.96 | 0.075 | 0.496 |
| Mexico Midget | Jul. 8, 2010 | 0.369 | 0.907 | 2.48 | 0.002 | 1.800 |
| Lemon Drop | Jul. 8, 2010 | 0.776 | 0.382 | 0.87 | 0.012 | 1.398 |
| LA1482 | Jul. 8, 2010 | 0.044 | 0.039 | 0.67 | 0.367 | 0.036 |
| Matt's Wild Cherry | Jul. 8, 2010 | 0.251 | 0.018 | 0.92 | 0.004 | 0.221 |

| | | 1-nitro-2- | benzyl | | |
|---|---|---|---|---|---|
| | salicylaldehyde | phenylethane | alcohol | methylsalicylate | guaiacol |
| Variety | ng/gfw/hr | ng/gfw/hr | ng/gfw/hr | ng/gfw/hr | ng/gfw/hr |
| Supermarket Round | 0.52 | 2.170 | 0.51 | 0.812 | 0.797 |
| Three Sisters | 0.93 | 1.635 | 1.04 | 0.834 | 0.465 |
| Skorospelka Red | 0.10 | 0.457 | 0.61 | 0.006 | 0.203 |
| Yellow Perfection | 1.02 | 0.157 | 0.45 | 0.237 | 0.752 |
| Clear Pink Slicer | 0.90 | 0.568 | 0.73 | 0.406 | 0.437 |
| Tigerella Orange | 2.00 | 0.026 | 0.12 | 0.378 | 0.756 |
| Gulf State Market | 0.65 | 1.736 | 1.47 | 0.885 | 0.754 |
| Bloody Butcher | 0.53 | 0.474 | 0.39 | 0.129 | 0.434 |
| Garden Peach | 0.91 | 0.435 | 0.38 | 0.321 | 0.391 |
| Amish Salad | 0.68 | 0.411 | 0.30 | 0.011 | 0.036 |
| Ailsa Craig | 1.45 | 0.356 | 0.24 | 0.398 | 0.941 |
| Supermarket Round | 0.82 | 1.432 | 0.79 | 0.741 | 0.881 |
| Super Sioux | 0.69 | 0.898 | 0.50 | 1.680 | 0.646 |
| St. Pierre | 1.14 | 0.414 | 0.30 | 2.470 | 0.656 |
| Supermarket Round | 0.13 | 0.877 | 0.18 | 0.497 | 0.186 |
| Kentucky Beefsteak | 0.06 | 0.485 | 0.10 | 0.017 | 0.015 |
| Green Zebra | 0.82 | 0.258 | 1.08 | 0.706 | 0.721 |
| Zapotec | 0.41 | 0.799 | 0.28 | 0.346 | 0.710 |
| Supermarket Cherry | 0.15 | 3.966 | 0.19 | 0.011 | 0.024 |
| Yellow Jelly Bean | 0.35 | 1.236 | 2.55 | 0.113 | 0.074 |
| Mexico Midget | 0.03 | 4.026 | 1.69 | 0.003 | 0.031 |
| Lemon Drop | 0.19 | 1.174 | 1.38 | 1.150 | 0.719 |
| LA1482 | 0.19 | 0.135 | 0.97 | 1.058 | 0.072 |
| Matt's Wild Cherry | 0.05 | 0.865 | 1.46 | 0.024 | 0.124 |

TABLE 8X

| | | benzyl | | | | 2-phenyl |
|---|---|---|---|---|---|---|
| | | cyanide | Phenylacetaldehyde | benzaldehyde | eugenol | ethanol |
| Variety | panel date | ng/gfw/hr | ng/gfw/hr | ng/gfw/hr | ng/gfw/hr | ng/gfw/hr |
| Maglia Rosa Cherry | Nov. 3, 2010 | 0.033 | 0.179 | 3.56 | 1.025 | 0.174 |
| Wisconsin 55 | Nov. 3, 2010 | 0.090 | 0.395 | 2.59 | 1.394 | 0.918 |
| Glacier | Nov. 3, 2010 | 0.019 | 0.172 | 3.20 | 0.965 | 0.246 |
| Oregon Spring Bush | Nov. 3, 2010 | 0.042 | 0.275 | 1.14 | 0.834 | 0.481 |
| Santiam | Nov. 3, 2010 | 0.041 | 0.213 | 2.53 | 1.248 | 0.203 |
| Giant Oxheart | Nov. 3, 2010 | 0.256 | 0.259 | 2.04 | 3.248 | 0.354 |
| Micado Violettor | Nov. 12, 2010 | 0.014 | 0.083 | 1.32 | 1.049 | 0.008 |
| Livingston's Globe | Nov. 12, 2010 | 0.016 | 0.102 | 1.03 | 0.832 | 0.012 |
| Old Brooks | Nov. 12, 2010 | 0.041 | 0.128 | 2.60 | 1.609 | 0.017 |
| Park's Whopper | Nov. 12, 2010 | 0.016 | 0.058 | 1.04 | 1.057 | 0.011 |
| Hillbilly | Nov. 12, 2010 | 0.009 | 0.100 | 1.46 | 0.008 | 0.008 |
| German Head | Nov. 12, 2010 | 0.016 | 0.075 | 0.86 | 0.008 | 0.043 |
| Livingston's Stone | Nov. 17, 2010 | 0.018 | 0.068 | 0.41 | 0.478 | 0.021 |
| Ponderosa Pink | Nov. 17, 2010 | 0.051 | 0.140 | 0.71 | 0.008 | 0.570 |
| Cherokee Purple | Nov. 17, 2010 | 0.016 | 0.090 | 0.54 | 1.065 | 0.083 |
| Black | Nov. 17, 2010 | 0.016 | 0.057 | 0.34 | 0.674 | 0.021 |
| Nyagous | Nov. 17, 2010 | 0.017 | 0.122 | 0.49 | 3.395 | 0.012 |
| Oaxacan Pink | Nov. 17, 2010 | 0.030 | 0.073 | 0.73 | 2.636 | 0.244 |
| Livingston's Golden Queen | Nov. 23, 2010 | 0.011 | 0.090 | 1.38 | 0.650 | 0.003 |
| Ponderosa Red | Nov. 23, 2010 | 0.015 | 0.060 | 0.58 | 1.940 | 0.015 |
| Japanese Black Trifele | Nov. 23, 2010 | 0.011 | 0.064 | 0.37 | 0.609 | 0.008 |
| Crimson Sprinter | Nov. 23, 2010 | 0.010 | 0.087 | 1.51 | 1.927 | 0.004 |
| Legend | Nov. 23, 2010 | 0.009 | 0.115 | 1.16 | 0.028 | 0.132 |
| Zapotec | Nov. 23, 2010 | 0.018 | 0.223 | 1.14 | 0.729 | 0.024 |
| Sunray | Dec. 1, 2010 | 0.010 | 0.070 | 1.64 | 0.680 | 0.025 |
| Martian Giant | Dec. 1, 2010 | 0.009 | 0.037 | 0.72 | 0.007 | 0.009 |
| Rose Tomato | Dec. 1, 2010 | 0.031 | 0.114 | 0.48 | 0.020 | 0.016 |
| Mr. Stripey | Dec. 1, 2010 | 0.023 | 0.087 | 0.73 | 0.444 | 0.104 |
| Tasti-Lee | Dec. 1, 2010 | 0.008 | 0.052 | 0.41 | 0.453 | 0.005 |
| Costoluto Genovese | Dec. 1, 2010 | 0.027 | 0.089 | 1.32 | 1.621 | 0.008 |

| | | 1-nitro-2- | benzyl | | |
|---|---|---|---|---|---|
| | salicylaldehyde | phenylethane | alcohol | methylsalicylate | guaiacol |
| Variety | ng/gfw/hr | ng/gfw/hr | ng/gfw/hr | ng/gfw/hr | ng/gfw/hr |
| Maglia Rosa Cherry | 1.08 | 0.085 | 0.43 | 0.304 | 0.698 |
| Wisconsin 55 | 1.32 | 0.925 | 0.61 | 0.873 | 1.577 |
| Glacier | 0.90 | 0.087 | 0.51 | 0.298 | 0.380 |
| Oregon Spring Bush | 1.34 | 0.314 | 0.26 | 0.322 | 0.938 |
| Santiam | 1.82 | 0.163 | 0.32 | 0.943 | 1.224 |
| Giant Oxheart | 2.39 | 0.613 | 0.66 | 0.385 | 1.304 |
| Micado Violettor | 1.24 | 0.014 | 0.11 | 0.228 | 0.825 |
| Livingston's Globe | 1.05 | 0.047 | 0.09 | 1.668 | 0.888 |
| Old Brooks | 1.75 | 0.262 | 0.49 | 2.010 | 1.388 |
| Park's Whopper | 1.43 | 0.031 | 0.16 | 0.470 | 0.702 |
| Hillbilly | 0.30 | 0.103 | 0.28 | 0.025 | 0.070 |
| German Head | 0.49 | 0.120 | 0.34 | 0.015 | 0.048 |
| Livingston's Stone | 1.22 | 0.015 | 0.10 | 0.090 | 0.646 |
| Ponderosa Pink | 0.24 | 0.163 | 0.21 | 0.026 | 0.088 |
| Cherokee Purple | 1.46 | 0.026 | 0.42 | 0.268 | 0.892 |
| Black | 1.15 | 0.029 | 0.31 | 0.134 | 0.669 |
| Nyagous | 1.75 | 0.013 | 0.09 | 0.340 | 1.020 |
| Oaxacan Pink | 0.79 | 0.049 | 0.52 | 0.798 | 0.827 |
| Livingston's Golden Queen | 1.17 | 0.012 | 0.09 | 0.483 | 1.567 |
| Ponderosa Red | 1.67 | 0.016 | 0.34 | 0.127 | 0.671 |
| Japanese Black Trifele | 1.68 | 0.012 | 0.05 | 0.065 | 0.468 |
| Crimson Sprinter | 1.09 | 0.012 | 0.20 | 0.272 | 0.495 |
| Legend | 0.28 | 0.038 | 0.13 | 0.019 | 0.086 |
| Zapotec | 1.94 | 0.020 | 0.25 | 0.064 | 0.980 |
| Sunray | 1.35 | 0.040 | 0.10 | 0.124 | 0.568 |
| Martian Giant | 0.15 | 0.033 | 0.12 | 0.007 | 0.051 |
| Rose Tomato | 0.46 | 0.030 | 0.36 | 0.009 | 0.123 |
| Mr. Stripey | 0.95 | 0.212 | 0.14 | 0.052 | 0.572 |
| Tasti-Lee | 0.89 | 0.038 | 0.07 | 0.667 | 0.398 |
| Costoluto Genovese | 0.96 | 0.015 | 0.52 | 0.222 | 0.803 |

TABLE 8Y

| | | | 2,5-dimethyl-4- | | | | |
|---|---|---|---|---|---|---|---|
| | | | hydroxy-3(2H)- | | | | |
| | | methional | furanone | 4-carene | 2-ethylfuran | benzothiazole | p-anisaldehyde |
| Variety | panel date | ng/gfw/hr | ng/gfw/hr | ng/gfw/hr | ng/gfw/hr | ng/gfw/hr | ng/gfw/hr |
| Cherry Roma | Feb. 24, 2010 | 0.501 | 0.794 | 0.022 | 0.11 | 0.097 | 0.004 |
| Ailsa Craig | Feb. 24, 2010 | 0.548 | 0.466 | 0.023 | 0.19 | 0.064 | 0.005 |
| Red Pear | Feb. 24, 2010 | 0.148 | 0.190 | 0.017 | 0.07 | 0.075 | 0.003 |
| Tommy Toe | Feb. 24, 2010 | 0.306 | 0.288 | 0.024 | 0.09 | 0.052 | 0.009 |
| Supermarket Cherry | Feb. 24, 2010 | 0.070 | 0.080 | 0.009 | 0.06 | 0.087 | 0.002 |
| Porter | Feb. 24, 2010 | 0.087 | 0.376 | 0.017 | 0.05 | 0.068 | 0.003 |
| Matina | Mar. 2, 2010 | 0.196 | 1.402 | 0.056 | 0.06 | 0.110 | 0.021 |
| Bloody Butcher | Mar. 2, 2010 | 0.089 | 0.138 | 0.099 | 0.09 | 0.121 | 0.003 |
| Chadwick Cherry | Mar. 2, 2010 | 0.177 | 0.837 | 0.040 | 0.08 | 0.050 | 0.015 |
| Thai Pink Cherry | Mar. 2, 2010 | 0.107 | 0.258 | 0.024 | 0.21 | 0.085 | 0.009 |
| Peacevine Cherry | Mar. 2, 2010 | 0.496 | 0.486 | 0.114 | 0.12 | 0.120 | 0.003 |
| Supermarket Cherry | Mar. 2, 2010 | 0.140 | 0.114 | 0.010 | 0.10 | 0.056 | 0.003 |
| Stupice | Mar. 2, 2010 | 0.216 | 0.348 | 0.025 | 0.20 | 0.066 | 0.005 |
| Large Red Cherry | Mar. 2, 2010 | 0.509 | 0.427 | 0.031 | 0.12 | 0.089 | 0.006 |
| Bloody Butcher | Mar. 18, 2010 | 0.105 | 0.262 | 0.087 | 0.09 | 0.033 | 0.008 |
| Giant Belgium | Mar. 18, 2010 | 0.194 | 0.455 | 0.042 | 0.25 | 0.034 | 0.010 |
| Thessaloniki | Mar. 18, 2010 | 0.108 | 0.115 | 0.016 | 0.15 | 0.045 | 0.005 |
| Supermarket Round | Mar. 18, 2010 | 0.076 | 0.153 | 0.019 | 0.11 | 0.043 | 0.012 |
| Marmande VFA | Mar. 18, 2010 | 0.058 | 0.060 | 0.108 | 0.14 | 0.071 | 0.009 |
| Red Calabash | Apr. 1, 2010 | 0.189 | 0.325 | 0.048 | 0.10 | 0.063 | 0.003 |
| Brandywine | Apr. 1, 2010 | 0.052 | 0.489 | 0.070 | 0.12 | 0.042 | 0.002 |
| Dixie Golden Giant | Apr. 1, 2010 | 0.044 | 0.180 | 0.082 | 0.09 | 0.099 | 0.005 |
| Aunt Ruby's German Green | Apr. 1, 2010 | 0.181 | 0.527 | 0.047 | 0.13 | 0.057 | 0.003 |
| Thessaloniki | Apr. 1, 2010 | 0.066 | 0.123 | 0.011 | 0.08 | 0.052 | 0.003 |
| Supermarket Round | Apr. 1, 2010 | 0.027 | 0.166 | 0.011 | 0.12 | 0.097 | 0.003 |

TABLE 8Z

| | | | 2,5-dimethyl-4- | | | | |
|---|---|---|---|---|---|---|---|
| | | | hydroxy-3(2H)- | | | | |
| | | methional | furanone | 4-carene | 2-ethylfuran | benzothiazole | p-anisaldehyde |
| Variety | panel date | ng/gfw/hr | ng/gfw/hr | ng/gfw/hr | ng/gfw/hr | ng/gfw/hr | ng/gfw/hr |
| Supermarket Round | Jun. 11, 2010 | 0.116 | 0.116 | 0.016 | 0.07 | 0.014 | 0.008 |
| Three Sisters | Jun. 11, 2010 | 0.150 | 0.443 | 0.013 | 0.06 | 0.040 | 0.016 |
| Skorospelka Red | Jun. 11, 2010 | 0.131 | 0.200 | 0.009 | 0.14 | 0.074 | 0.015 |
| Yellow Perfection | Jun. 11, 2010 | 0.167 | 0.290 | 0.017 | 0.14 | 0.018 | 0.004 |
| Clear Pink Slicer | Jun. 11, 2010 | 0.181 | 0.244 | 0.015 | 0.13 | 0.018 | 0.006 |
| Tigerella Orange | Jun. 11, 2010 | 0.037 | 0.270 | 0.012 | 0.22 | 0.009 | 0.007 |
| Gulf State Market | Jun. 17, 2010 | 0.094 | 0.308 | 0.006 | 0.09 | 0.051 | 0.018 |
| Bloody Butcher | Jun. 17, 2010 | 0.039 | 0.456 | 0.009 | 0.04 | 0.042 | 0.013 |
| Garden Peach | Jun. 17, 2010 | 0.086 | 0.067 | 0.007 | 0.02 | 0.079 | 0.001 |
| Amish Salad | Jun. 17, 2010 | 0.048 | 0.281 | 0.008 | 0.04 | 0.028 | 0.008 |
| Ailsa Craig | Jun. 17, 2010 | 0.049 | 0.971 | 0.012 | 0.08 | 0.033 | 0.009 |
| Supermarket Round | Jun. 17, 2010 | 0.172 | 0.061 | 0.009 | 0.03 | 0.023 | 0.001 |
| Super Sioux | Jun. 30, 2010 | 0.107 | 0.298 | 0.006 | 0.06 | 0.044 | 0.001 |
| St. Pierre | Jun. 30, 2010 | 0.080 | 0.294 | 0.010 | 0.10 | 0.027 | 0.006 |
| Supermarket Round | Jun. 30, 2010 | 0.099 | 0.061 | 0.005 | 0.08 | 0.031 | 0.001 |
| Kentucky Beefsteak | Jun. 30, 2010 | 0.023 | 0.169 | 0.003 | 0.03 | 0.035 | 0.002 |
| Green Zebra | Jun. 30, 2010 | 0.148 | 0.271 | 0.015 | 0.14 | 0.029 | 0.002 |
| Zapotec | Jun. 30, 2010 | 0.137 | 0.053 | 0.006 | 0.14 | 0.039 | 0.029 |
| Supermarket Cherry | Jul. 8, 2010 | 0.157 | 0.302 | 0.008 | 0.02 | 0.034 | 0.002 |
| Yellow Jelly Bean | Jul. 8, 2010 | 0.106 | 0.123 | 0.010 | 0.02 | 0.032 | 0.001 |
| Mexico Midget | Jul. 8, 2010 | 0.247 | 0.191 | 0.005 | 0.09 | 0.035 | 0.002 |
| Lemon Drop | Jul. 8, 2010 | 0.054 | 0.216 | 0.016 | 0.09 | 0.045 | 0.001 |
| LA1482 | Jul. 8, 2010 | 0.087 | 0.178 | 0.018 | 0.13 | 0.035 | 0.006 |
| Matt's Wild Cherry | Jul. 8, 2010 | 0.050 | 0.143 | 0.045 | 0.01 | 0.058 | 0.008 |

TABLE 8AA

| | | | 2,5-dimethyl-4-hydroxy- | | | | |
|---|---|---|---|---|---|---|---|
| | | methional | 3(2H)-furanone | 4-carene | 2-ethylfuran | benzothiazole | p-anisaldehyde |
| Variety | panel date | ng/gfw/hr | ng/gfw/hr | ng/gfw/hr | ng/gfw/hr | ng/gfw/hr | ng/gfw/hr |
| Maglia Rosa Cherry | Nov. 3, 2010 | 0.138 | 0.115 | 0.021 | 0.32 | 0.091 | 0.002 |
| Wisconsin 55 | Nov. 3, 2010 | 0.374 | 0.059 | 0.017 | 0.13 | 0.105 | 0.006 |
| Glacier | Nov. 3, 2010 | 0.103 | 0.067 | 0.017 | 0.10 | 0.070 | 0.069 |
| Oregon Spring Bush | Nov. 3, 2010 | 0.114 | 0.113 | 0.013 | 0.15 | 0.087 | 0.003 |
| Santiam | Nov. 3, 2010 | 0.117 | 0.102 | 0.018 | 0.13 | 0.081 | 0.004 |
| Giant Oxheart | Nov. 3, 2010 | 0.080 | 0.210 | 0.040 | 0.10 | 0.076 | 0.008 |
| Micado Violettor | Nov. 12, 2010 | 0.138 | 0.177 | 0.006 | 0.07 | 0.082 | 0.003 |
| Livingston's Globe | Nov. 12, 2010 | 0.073 | 0.063 | 0.005 | 0.15 | 0.089 | 0.002 |
| Old Brooks | Nov. 12, 2010 | 0.109 | 0.247 | 0.008 | 0.18 | 0.087 | 0.019 |
| Park's Whopper | Nov. 12, 2010 | 0.046 | 0.091 | 0.011 | 0.26 | 0.055 | 0.005 |
| Hillbilly | Nov. 12, 2010 | 0.132 | 0.153 | 0.004 | 0.09 | 0.056 | 0.015 |
| German Head | Nov. 12, 2010 | 0.118 | 0.113 | 0.014 | 0.20 | 0.072 | 0.006 |
| Livingston's Stone | Nov. 17, 2010 | 0.063 | 0.086 | 0.015 | 0.08 | 0.090 | 0.003 |
| Ponderosa Pink | Nov. 17, 2010 | 0.106 | 0.043 | 0.030 | 0.15 | 0.065 | 0.006 |
| Cherokee Purple | Nov. 17, 2010 | 0.105 | 0.107 | 0.017 | 0.07 | 0.082 | 0.006 |
| Black | Nov. 17, 2010 | 0.063 | 0.083 | 0.014 | 0.06 | 0.084 | 0.012 |
| Nyagous | Nov. 17, 2010 | 0.054 | 0.151 | 0.044 | 0.04 | 0.063 | 0.002 |
| Oaxacan Pink | Nov. 17, 2010 | 0.099 | 0.324 | 0.007 | 0.07 | 0.046 | 0.004 |
| Livingston's Golden Queen | Nov. 23, 2010 | 0.128 | 0.157 | 0.019 | 0.19 | 0.134 | 0.004 |
| Ponderosa Red | Nov. 23, 2010 | 0.048 | 0.025 | 0.016 | 0.16 | 0.097 | 0.006 |
| Japanese Black Trifele | Nov. 23, 2010 | 0.026 | 0.019 | 0.012 | 0.06 | 0.079 | 0.004 |
| Crimson Sprinter | Nov. 23, 2010 | 0.073 | 0.070 | 0.015 | 0.19 | 0.144 | 0.004 |
| Legend | Nov. 23, 2010 | 0.081 | 0.055 | 0.018 | 0.17 | 0.123 | 0.003 |
| Zapotec | Nov. 23, 2010 | 0.198 | 0.018 | 0.012 | 0.20 | 0.080 | 0.022 |
| Sunray | Dec. 1, 2010 | 0.077 | 0.031 | 0.021 | 0.10 | 0.077 | 0.002 |
| Martian Giant | Dec. 1, 2010 | 0.075 | 0.015 | 0.033 | 0.12 | 0.083 | 0.004 |
| Rose Tomato | Dec. 1, 2010 | 0.068 | 0.071 | 0.034 | 0.18 | 0.096 | 0.003 |
| Mr. Stripey | Dec. 1, 2010 | 0.115 | 0.081 | 0.016 | 0.03 | 0.050 | 0.002 |
| Tasti-Lee | Dec. 1, 2010 | 0.050 | 0.025 | 0.017 | 0.26 | 0.069 | 0.004 |
| Costoluto Genovese | Dec. 1, 2010 | 0.073 | 0.015 | 0.035 | 0.25 | 0.126 | 0.003 |

TABLE 8BB

| | | | | | trans,trans- | | | |
|---|---|---|---|---|---|---|---|---|
| | | | nonyl | cis-4- | 2,4- | 1-octen-3- | propyl | prenyl |
| | panel | heptaldehyde | aldehyde | decenal | decadienal | one | acetate | acetate |
| Variety | date | ng/gfw/hr | ng/gfw/hr | ng/gfw/hr | ng/gfw/hr | ng/gfw/hr | ng/gfw/hr | ng/gfw/hr |
| Cherry Roma | Feb. 24, 2010 | 3.55 | 1.13 | 2.78 | 0.0176 | 0.141 | 0.08 | 0.0242 |
| Ailsa Craig | Feb. 24, 2010 | 4.56 | 0.30 | 1.09 | 0.0072 | 0.088 | 0.36 | 0.0187 |
| Red Pear | Feb. 24, 2010 | 2.58 | 0.31 | 0.63 | 0.0020 | 0.068 | 0.10 | 0.0143 |
| Tommy Toe | Feb. 24, 2010 | 3.48 | 0.30 | 0.90 | 0.0040 | 0.083 | 0.18 | 0.0193 |
| Supermarket Cherry | Feb. 24, 2010 | 1.04 | 0.25 | 0.52 | 0.0026 | 0.079 | 0.03 | 0.0145 |
| Porter | Feb. 24, 2010 | 1.49 | 0.24 | 1.83 | 0.0036 | 0.075 | 0.19 | 0.0129 |
| Matina | Mar. 2, 2010 | 9.50 | 0.30 | 1.81 | 0.0749 | 0.117 | 0.94 | 0.0382 |
| Bloody Butcher | Mar. 2, 2010 | 4.19 | 0.31 | 0.82 | 0.0011 | 0.043 | 0.23 | 0.0114 |
| Chadwick Cherry | Mar. 2, 2010 | 5.30 | 0.38 | 1.41 | 0.0136 | 0.091 | 0.17 | 0.0323 |
| Thai Pink Cherry | Mar. 2, 2010 | 3.45 | 0.40 | 1.33 | 0.0039 | 0.065 | 0.21 | 0.0183 |
| Peacevine Cherry | Mar. 2, 2010 | 3.72 | 0.67 | 1.92 | 0.0071 | 0.201 | 0.35 | 0.0180 |
| Supermarket Cherry | Mar. 2, 2010 | 0.77 | 0.24 | 0.55 | 0.0045 | 0.059 | 0.03 | 0.0147 |
| Stupice | Mar. 2, 2010 | 10.14 | 0.43 | 0.98 | 0.0111 | 0.051 | 0.31 | 0.0339 |
| Large Red Cherry | Mar. 2, 2010 | 7.99 | 0.28 | 1.17 | 0.0036 | 0.130 | 0.28 | 0.0278 |
| Bloody Butcher | Mar. 18, 2010 | 7.04 | 0.33 | 1.50 | 0.0117 | 0.063 | 0.22 | 0.0273 |
| Giant Belgium | Mar. 18, 2010 | 7.01 | 0.38 | 2.73 | 0.0066 | 0.058 | 0.29 | 0.0275 |
| Thessaloniki | Mar. 18, 2010 | 29.08 | 0.19 | 1.49 | 0.0056 | 0.038 | 0.14 | 0.0148 |
| Supermarket Round | Mar. 18, 2010 | 2.41 | 0.20 | 0.38 | 0.0087 | 0.021 | 0.72 | 0.0039 |
| Marmande VFA | Mar. 18, 2010 | 1.03 | 0.23 | 0.46 | 0.0071 | 0.017 | 0.07 | 0.0072 |
| Red Calabash | Apr. 1, 2010 | 4.39 | 0.54 | 0.90 | 0.0037 | 0.066 | 0.28 | 0.0153 |
| Brandywine | Apr. 1, 2010 | 1.13 | 0.37 | 0.95 | 0.0039 | 0.075 | 0.16 | 0.0161 |
| Dixie Golden Giant | Apr. 1, 2010 | 0.63 | 0.32 | 0.77 | 0.0040 | 0.071 | 0.40 | 0.0093 |
| Aunt Ruby's German Green | Apr. 1, 2010 | 7.15 | 0.48 | 1.59 | 0.0024 | 0.067 | 0.18 | 0.0151 |
| Thessaloniki | Apr. 1, 2010 | 27.01 | 0.15 | 0.86 | 0.0059 | 0.029 | 0.12 | 0.0069 |
| Supermarket Round | Apr. 1, 2010 | 0.85 | 0.33 | 0.51 | 0.0029 | 0.028 | 0.11 | 0.0053 |

TABLE 8CC

| | | | | | trans,trans- | | | |
|---|---|---|---|---|---|---|---|---|
| | | | nonyl | cis-4- | 2,4- | 1-octen- | propyl | prenyl |
| | panel | heptaldehyde | aldehyde | decenal | decadienal | 3-one | acetate | acetate |
| Variety | date | ng/gfw/hr | ng/gfw/hr | ng/gfw/hr | ng/gfw/hr | ng/gfw/hr | ng/gfw/hr | ng/gfw/hr |
| Supermarket Round | Jun. 11, 2010 | 1.55 | 0.26 | 1.34 | 0.0032 | 0.054 | 0.26 | 0.0061 |
| Three Sisters | Jun. 11, 2010 | 2.85 | 0.26 | 1.02 | 0.0214 | 0.032 | 0.76 | 0.0083 |
| Skorospelka Red | Jun. 11, 2010 | 2.48 | 0.29 | 0.46 | 0.0083 | 0.039 | 0.33 | 0.0157 |
| Yellow Perfection | Jun. 11, 2010 | 2.39 | 0.19 | 0.79 | 0.0075 | 0.033 | 0.34 | 0.0075 |
| Clear Pink Slicer | Jun. 11, 2010 | 1.39 | 0.16 | 0.98 | 0.0063 | 0.026 | 0.58 | 0.0052 |
| Tigerella Orange | Jun. 11, 2010 | 0.87 | 0.20 | 0.86 | 0.0069 | 0.031 | 0.41 | 0.0063 |
| Gulf State Market | Jun. 17, 2010 | 2.02 | 0.14 | 1.09 | 0.0074 | 0.035 | 0.33 | 0.0056 |
| Bloody Butcher | Jun. 17, 2010 | 1.11 | 0.20 | 0.98 | 0.0372 | 0.051 | 0.18 | 0.0114 |
| Garden Peach | Jun. 17, 2010 | 0.42 | 0.08 | 0.35 | 0.0033 | 0.017 | 0.15 | 0.0031 |
| Amish Salad | Jun. 17, 2010 | 0.71 | 0.12 | 0.90 | 0.0047 | 0.038 | 0.14 | 0.0083 |
| Ailsa Craig | Jun. 17, 2010 | 1.00 | 0.20 | 1.11 | 0.0572 | 0.086 | 0.33 | 0.0083 |
| Supermarket Round | Jun. 17, 2010 | 1.17 | 0.24 | 1.32 | 0.0082 | 0.033 | 1.04 | 0.1891 |
| Super Sioux | Jun. 30, 2010 | 1.83 | 0.10 | 0.90 | 0.0117 | 0.022 | 0.23 | 0.0045 |
| St. Pierre | Jun. 30, 2010 | 0.97 | 0.08 | 0.72 | 0.0116 | 0.040 | 0.18 | 0.0081 |
| Supermarket Round | Jun. 30, 2010 | 2.53 | 0.09 | 0.93 | 0.0039 | 0.017 | 0.18 | 0.0051 |
| Kentucky Beefsteak | Jun. 30, 2010 | 0.31 | 0.06 | 0.72 | 0.0128 | 0.018 | 0.16 | 0.0023 |
| Green Zebra | Jun. 30, 2010 | 1.97 | 0.07 | 0.43 | 0.0075 | 0.037 | 0.12 | 0.0027 |
| Zapotec | Jun. 30, 2010 | 0.69 | 0.11 | 0.50 | 0.0035 | 0.012 | 0.22 | 0.0058 |
| Supermarket Cherry | Jul. 8, 2010 | 1.51 | 0.17 | 1.57 | 0.0043 | 0.047 | 0.04 | 0.0113 |
| Yellow Jelly Bean | Jul. 8, 2010 | 3.99 | 0.21 | 0.58 | 0.0029 | 0.035 | 0.04 | 0.0024 |
| Mexico Midget | Jul. 8, 2010 | 8.73 | 0.27 | 0.50 | 0.0031 | 0.047 | 0.06 | 0.0035 |
| Lemon Drop | Jul. 8, 2010 | 2.24 | 0.15 | 0.53 | 0.0063 | 0.020 | 0.13 | 0.0032 |
| LA1482 | Jul. 8, 2010 | 1.93 | 0.21 | 0.24 | 0.0050 | 0.061 | 0.34 | 0.0079 |
| Matt's Wild Cherry | Jul. 8, 2010 | 1.58 | 0.27 | 0.26 | 0.0172 | 0.064 | 0.25 | 0.0008 |

TABLE 8DD

| | | | nonyl | cis-4- | trans,trans-2,4- | 1-octen-3- | propyl | prenyl |
|---|---|---|---|---|---|---|---|---|
| | panel | heptaldehyde | aldehyde | decenal | decadienal | one | acetate | acetate |
| Variety | date | ng/gfw/hr | ng/gfw/hr | ng/gfw/hr | ng/gfw/hr | ng/gfw/hr | ng/gfw/hr | ng/gfw/hr |
| Maglia Rosa Cherry | Nov. 3, 2010 | 1.58 | 0.20 | 0.41 | 0.0032 | 0.062 | 0.33 | 0.0137 |
| Wisconsin 55 | Nov. 3, 2010 | 6.04 | 0.20 | 1.31 | 0.0041 | 0.040 | 0.94 | 0.0549 |
| Glacier | Nov. 3, 2010 | 0.73 | 0.13 | 0.33 | 0.0023 | 0.031 | 0.76 | 0.0262 |
| Oregon Spring Bush | Nov. 3, 2010 | 1.16 | 0.21 | 0.78 | 0.0033 | 0.058 | 0.36 | 0.0131 |
| Santiam | Nov. 3, 2010 | 1.03 | 0.14 | 0.44 | 0.0028 | 0.041 | 0.55 | 0.0438 |
| Giant Oxheart | Nov. 3, 2010 | 1.51 | 0.18 | 0.98 | 0.0051 | 0.042 | 0.46 | 0.0228 |
| Micado Violettor | Nov. 12, 2010 | 0.74 | 0.14 | 1.02 | 0.0048 | 0.023 | 0.38 | 0.0104 |
| Livingston's Globe | Nov. 12, 2010 | 0.88 | 0.14 | 0.71 | 0.0024 | 0.021 | 0.19 | 0.0044 |
| Old Brooks | Nov. 12, 2010 | 6.42 | 0.21 | 1.65 | 0.0024 | 0.037 | 0.13 | 0.0070 |
| Park's Whopper | Nov. 12, 2010 | 1.13 | 0.15 | 0.91 | 0.0027 | 0.029 | 0.26 | 0.0152 |
| Hillbilly | Nov. 12, 2010 | 1.73 | 0.17 | 0.79 | 0.0030 | 0.033 | 0.21 | 0.0076 |
| German Head | Nov. 12, 2010 | 1.57 | 0.15 | 1.23 | 0.0040 | 0.022 | 0.22 | 0.0067 |
| Livingston's Stone | Nov. 17, 2010 | 0.78 | 0.20 | 0.80 | 0.0033 | 0.030 | 0.47 | 0.0721 |
| Ponderosa Pink | Nov. 17, 2010 | 2.08 | 0.31 | 0.61 | 0.0031 | 0.048 | 0.45 | 0.0324 |
| Cherokee Purple | Nov. 17, 2010 | 1.12 | 0.29 | 0.98 | 0.0055 | 0.033 | 0.60 | 0.0060 |
| Black | Nov. 17, 2010 | 0.58 | 0.16 | 0.61 | 0.0067 | 0.026 | 0.20 | 0.0123 |
| Nyagous | Nov. 17, 2010 | 0.96 | 0.32 | 1.07 | 0.0048 | 0.056 | 0.45 | 0.0071 |
| Oaxacan Pink | Nov. 17, 2010 | 2.13 | 0.13 | 0.51 | 0.0174 | 0.411 | 0.61 | 0.0242 |
| Livingston's Golden Queen | Nov. 23, 2010 | 1.47 | 0.15 | 0.98 | 0.0068 | 0.031 | 0.21 | 0.0091 |
| Ponderosa Red | Nov. 23, 2010 | 0.66 | 0.17 | 0.55 | 0.0036 | 0.012 | 0.32 | 0.0289 |
| Japanese Black Trifele | Nov. 23, 2010 | 0.36 | 0.14 | 0.60 | 0.0034 | 0.018 | 0.14 | 0.0123 |
| Crimson Sprinter | Nov. 23, 2010 | 1.01 | 0.13 | 0.58 | 0.0045 | 0.019 | 0.35 | 0.1504 |
| Legend | Nov. 23, 2010 | 1.45 | 0.20 | 0.84 | 0.0048 | 0.024 | 0.42 | 0.0401 |
| Zapotec | Nov. 23, 2010 | 0.79 | 0.16 | 0.60 | 0.0052 | 0.016 | 0.29 | 0.0341 |
| Sunray | Dec. 1, 2010 | 1.07 | 0.14 | 0.38 | 0.0023 | 0.012 | 0.26 | 0.0094 |
| Martian Giant | Dec. 1, 2010 | 3.88 | 0.12 | 0.49 | 0.0022 | 0.019 | 0.25 | 0.0061 |
| Rose Tomato | Dec. 1, 2010 | 1.15 | 0.20 | 0.85 | 0.0018 | 0.028 | 0.22 | 0.0171 |
| Mr. Stripey | Dec. 1, 2010 | 1.85 | 0.08 | 0.55 | 0.0036 | 0.016 | 0.32 | 0.0060 |
| Tasti-Lee | Dec. 1, 2010 | 1.77 | 0.15 | 0.61 | 0.0009 | 0.019 | 0.20 | 0.0064 |
| Costoluto Genovese | Dec. 1, 2010 | 1.47 | 0.27 | 0.35 | 0.0024 | 0.032 | 0.47 | 0.1483 |

1. A method of identifying a hybrid tomato plant that produces better-tasting fruit comprising the steps of:
providing tomato samples from a plurality of different tomato plant varieties to a tasting panel;
accumulating results of the tasting panel, wherein each panel member assigns a liking score to each tomato tested;
performing a chemical analysis of a tomato from each of the variety of tomatoes tested by the panel, comprising quantifying an amount of a plurality of flavor-associated compounds from each tomato, wherein the flavor-associated compounds are selected from the group of compounds consisting of: sugars, acids, volatile compounds, and combinations thereof and wherein at least one of the flavor-associated compounds quantified is a volatile compound;
correlating the results of the tasting panel scores with the calculated amounts of flavor-associated compounds for each tomato from the chemical analysis to determine which volatile compounds are positively associated with liking and which volatile compounds are negatively associated with liking;
determining criteria for a better-tasting tomato based on the correlations between liking scores and the chemical content of a tomato; and
identifying a hybrid tomato plant that produces fruit having at least one of the criterion for a better-tasting tomato.

2. The method of claim 1, wherein the volatile compound(s) are selected from the group of volatile compounds consisting of: 1-penten-3-one, trans-2-pentenal, trans-2-heptenal, trans-3-hexen-1-ol, trans-2-hexenal, cis-2-penten-1-ol, 6-methyl-5-hepten-2-ol, nonyl aldehyde, isovaleronitrile, cis-4-decenal, 3-methyl-1-butanol, 2,5-dimethyl-4-hydroxy-3(2H)-furanone, 1-pentanol, methional, benzyl cyanide, isovaleraldehyde, 3-pentanone, 2-isobutylthaizole, benzaldehyde, isovaleric acid, 1-nitro-3-methylbutane, β-ionone, β-cyclocitral, 6-methyl-5-hepten-2-one, geranial, phenylacetaldehyde, geranylacetone, 2-phenyl ethanol, 1-octen-3-one, eugenol, salicylaldehyde, isobutyl acetate, butyl acetate, and 2-methylbutanal.

3. The method of claim 2, wherein two or more of the volatile compounds are quantified.

4. The method of claim 2, wherein five or more of the volatile compounds are quantified.

5. A hybrid tomato plant that produces tomato fruit comprising a greater amount of at least one volatile compound positively associated with liking than the amount of that volatile compound in fruit produced by an ancestor elite tomato cultivar, wherein the volatile compound positively associated with liking is selected from the group of volatile compounds consisting of: 1-penten-3-one, trans-2-pentenal, trans-2-heptenal, trans-3-hexen-1-ol, trans-2-hexenal, cis-2-penten-1-ol, 6-methyl-5-hepten-2-ol, nonyl aldehyde, isovaleronitrile, cis-4-decenal, 3-methyl-1-butanol, 2,5-dimethyl-4-hydroxy-3(2H)-furanone, 1-pentanol, methional, benzyl cyanide, isovaleraldehyde, 3-pentanone, 2-isobutylthaizole, benzaldehyde, isovaleric acid, 1-nitro-3-methylbutane, β-ionone, β-cyclocitral, 6-methyl-5-hepten-2-one, geranial, phenylacetaldehyde, geranylacetone, 2-phenyl ethanol, 1-octen-3-one, and any combination of two or more of these volatile compounds.

6. The hybrid tomato plant of claim 5, wherein the volatile compound is selected from the group of volatile compounds consisting of: 1-penten-3-one, trans-2-hexenal, cis-2-penten-1-ol, geranial, 3-methyl-1-butanol, 1-octen-3-one, trans-2-pentenal, isovaleronitrile, trans-3-hexen-1-ol, 1-nitro-3-methylbutane, 6-methyl-5-hepten-2-one, and any combination of two or more of these volatile compounds.

7. The hybrid tomato plant of claim 5, wherein the plant produces tomato fruit comprising a greater amount of at least 2 of the volatile compounds than the amount of that volatile compound in fruit produced by an ancestor elite tomato cultivar.

8. The hybrid tomato plant of claim 5, wherein the plant produces tomato fruit comprising a greater amount of at least 5 of the volatile compounds than the amount of that volatile compound in fruit produced by an ancestor elite tomato cultivar.

9. The hybrid tomato plant of claim 5, wherein the tomato fruit produced by the hybrid tomato plant also comprises a lower amount in the fruit of the plant of at least one volatile compound negatively associated with liking than the amount of that volatile compound in fruit produced by an ancestor elite tomato cultivar, wherein the volatile compound negatively associated with liking is selected from the group of volatile compounds consisting of: eugenol, salicylaldehyde, isobutyl acetate, butyl acetate, 2-methylbutanal, and any combination of two or more of these volatile compounds.

10. The tomato plant of claim 9, wherein the plant produces tomato fruit comprising a lower amount of at least 2 of the volatile compounds than the amount of that volatile compound in fruit produced by an ancestor elite tomato cultivar.

11. The hybrid tomato plant of claim 5, wherein the tomato fruit produced by the hybrid tomato plant also comprises a higher sugar content than the ancestor elite tomato cultivar, wherein the sugar is glucose, or fructose, or a combination of glucose and fructose.

12. The hybrid tomato plant of claim 5, wherein the volatile compound is selected from one or more of the following compounds in the following amounts, measured as volatile emissions:
1-penten-3-one, having a volatile emission of about 1.6 ng gFW⁻¹h⁻¹ or more;
trans-2-pentenal, volatile emission of about 1.1 ng gFW⁻¹h⁻¹ or more;
trans-2-heptenal, having a volatile emission of about 0.44 ng gFW⁻¹h⁻¹ or more;
trans-3-hexen-1-ol, having a volatile emission of about 1.2 ng gFW⁻¹h⁻¹ or more;
6-methyl-5-hepten-2-ol having a volatile emission of about 0.17 ng gFW⁻¹h⁻¹ or more;
nonyl aldehyde, having a volatile emission of about 0.30 ng gFW⁻¹h⁻¹ or more;
isovaleronitrile, having a volatile emission of about 14.0 ng gFW⁻¹h⁻¹ or more;
cis-4-decenal, having a volatile emission of about 1.2 ng gFW⁻¹h⁻¹ or more;
3-methyl-1-butanol, having a volatile emission of about 39.1 ng gFW⁻¹h⁻¹ or more;
2,5-dimethyl-4-hydroxy-3(2H)-furanone, having a volatile emission of about 0.39 ng gFW⁻¹h⁻¹ or more;
1-pentanol, having a volatile emission of about 3.8 ng gFW⁻¹h⁻¹ or more;
methional, having a volatile emission of about 0.16 ng gFW⁻¹h⁻¹ or more;
benzyl cyanide, having a volatile emission of about 0.29 ng gFW⁻¹h⁻¹ or more;
isovaleraldehyde, having a volatile emission of about 14.1 ng gFW⁻¹h⁻¹ or more;
3-pentanone, having a volatile emission of about 6.6 ng gFW⁻¹h⁻¹ or more;
2-isobutylthaizole, having a volatile emission of about 5.9 ng gFW⁻¹h⁻¹ or more;
benzaldehyde, having a volatile emission of about 3.1 ng gFW⁻¹h⁻¹ or more;
isovaleric acid, having a volatile emission of about 0.08 ng gFW⁻¹h⁻¹ or more;
1-nitro-3-methylbutane, having a volatile emission of about 19.1 ng gFW⁻¹h⁻¹ or more;
β-ionone, having a volatile emission of about 0.06 ng gFW⁻¹h⁻¹ or more;
β-cyclocitral, having a volatile emission of about 0.10 ng gFW⁻¹h⁻¹ or more;
6-methyl-5-hepten-2-one, having a volatile emission of about 4.2 ng gFW⁻¹h⁻¹ or more;
geranial, having a volatile emission of about 0.15 ng gFW⁻¹h⁻¹ or more¹;
phenylacetaldehyde, having a volatile emission of about 0.29 ng gFW⁻¹h⁻¹ or more;
geranylacetone, having a volatile emission of about 1.61 ng gFW⁻¹h⁻¹ or more;
2-phenyl ethanol, having a volatile emission of about 0.7 ng gFW⁻¹h⁻¹ or more;
1-octen-3-one, having a content of about 0.06 ng gFW⁻¹h⁻¹ or more;
trans-2-hexenal, having a content of about 4.6 ng gFW⁻¹h⁻¹ or more; and
cis-2-penten-1-ol, having a content of about 1.2 ng gFW⁻¹h⁻¹ or more.

13. An F1 hybrid tomato plant produced by crossing an heirloom tomato cultivar and a parent of an elite hybrid cultivar, wherein the F1 hybrid tomato plant produces fruit that comprises a higher amount of at least one volatile compound positively associated with liking than the amount of that volatile compound present in fruit produced by the elite hybrid cultivar.

14. The F1 hybrid tomato plant of claim 13, wherein the at least one volatile compound positively associated with liking is selected from the group of volatile compounds consisting of: 1-penten-3-one, trans-2-pentenal, trans-2-heptenal, trans-3-hexen-1-ol, trans-2-hexenal, cis-2-penten-1-ol, 6-methyl-5-hepten-2-ol, nonyl aldehyde, isovaleronitrile, cis-4-decenal, 3-methyl-1-butanol, 2,5-dimethyl-4-hydroxy-3(2H)-furanone, 1-pentanol, methional, benzyl cyanide, isovaleraldehyde, 3-pentanone, 2-isobutylthaizole, benzaldehyde, isovaleric acid, 1-nitro-3-methylbutane, β-ionone, β-cyclocitral, 6-methyl-5-hepten-2-one, geranial, phenylacetaldehyde, geranylacetone, 2-phenyl ethanol, 1-octen-3-one, and any combination of two or more of these volatile compounds.

15. The F1 hybrid tomato plant of claim 13, wherein the at least one volatile compound positively associated with liking is selected from the group of volatile compounds consisting of: 1-penten-3-one, trans-2-hexenal, cis-2-penten-1-ol, geranial, 3-methyl-1-butanol, 1-octen-3-one, trans-2-pentenal, isovaleronitrile, trans-3-hexen-1-ol, 1-nitro-3-methylbutane, 6-methyl-5-hepten-2-one, and any combination of two or more of these volatile compounds.

16. The F1 hybrid tomato plant of claim 14, wherein the fruit of the F1 hybrid tomato plant comprises a higher amount of at least two volatile compounds positively associated with liking than the amount of those volatile compounds present in fruit produced by the elite hybrid cultivar.

17. The F1 hybrid tomato plant of claim 13, wherein the F1 hybrid tomato plant produces fruit that comprises a lower amount of at least one volatile compound negatively associated with liking than the amount of that volatile compound present in the fruit produced by the elite hybrid cultivar.

18. The F1 hybrid tomato plant of claim 17, wherein the at least one volatile compound negatively associated with liking is selected from the group of volatile compounds consisting of: eugenol, salicylaldehyde, isobutyl acetate, butyl acetate, 2-methylbutanal, and any combination of two or more of these volatile compounds.

19. The F1 hybrid tomato plant of claim 13, wherein the fruit of the F1 hybrid tomato plant further comprises a higher sugar content than fruit of the elite hybrid cultivar.

20. The F1 hybrid tomato plant of claim 13, wherein the fruit of the F1 hybrid tomato plant further comprises a sugar to acid ratio from about 8 to about 16.

21. The F1 hybrid tomato plant of claim 13, wherein the heirloom tomato cultivar is selected from the group of cultivars consisting of: Cherry Roma, Matina, Ailsa Craig, Red Calabash, Red Pear, Bloody Butcher, Maglia Rosa Cherry, Brandywine, Tommy Toe, Chadwick Cherry, Livingston's Stone, Super Sioux, St. Pierre, German Queen, Wisconsin 55, Micado Violettor, Livingston's Globe, and Gulf State Market.

22. A hybrid tomato plant that produces tomato fruit comprising the following volatile compounds in about the following amounts, measured as volatile emission (ng gFW⁻¹h⁻¹):
about 1.6 ng gFW⁻¹h⁻¹ or more of 1-penten-3-one,
about 1.1 ng gFW⁻¹h⁻¹ or more of trans-2-pentenal,
about 1.2 ng gFW⁻¹h⁻¹ or more of trans-3-hexen-1-ol,
about 14.0 ng gFW⁻¹h⁻¹ or more of isovaleronitrile,
about 39.1 ng gFW⁻¹h⁻¹ or more of 3-methyl-1-butanol,
about 19.1 ng gFW⁻¹h⁻¹ or more of 1-nitro-3-methylbutane,
about 4.2 ng gFW⁻¹h⁻¹ or more of 6-methyl-5-hepten-2-one,
about 0.15 ng gFW⁻¹h⁻¹ or more of geranial,
about 0.06 ng gFW⁻¹h⁻¹ or more of 1-octen-3-one,
about 4.6 ng gFW¹h⁻¹ or more of trans-2-hexenal,
about 1.2 ng gFW⁻¹h⁻¹ or more of cis-2-penten-1-ol,
about 0.48 ng gFW⁻¹h⁻¹ or less of eugenol,
about 3.9 ng gFW⁻¹h⁻¹ or less of 2-methylbutanal,
about 0.17 ng gFW⁻¹h⁻¹ or less of butylacetate, and
about 0.95 ng gFW⁻¹h⁻¹ or less of isobutylacetate.

23. A method of making a hybrid tomato plant comprising:
crossing a parent of an elite hybrid tomato cultivar with an heirloom tomato cultivar, wherein the heirloom tomato cultivar produces tomato fruit with a greater amount of at least one volatile compound positively associated with liking than the elite hybrid tomato cultivar, to produce an F1 hybrid tomato plant that produces tomato fruit with a greater amount of the at least one volatile compound positively associated with liking than the elite hybrid tomato cultivar.

24. The method of claim 23, wherein the at least one volatile compound positively associated with liking is selected from the group of volatile compounds consisting of: 1-penten-3-one, trans-2-pentenal, trans-2-heptenal, trans-3-hexen-1-ol, trans-2-hexenal, cis-2-penten-1-ol, 6-methyl-5-hepten-2-ol, nonyl aldehyde, isovaleronitrile, cis-4-decenal, 3-methyl-1-butanol, 2,5-dimethyl-4-hydroxy-3(2H)-furanone, 1-pentanol, methional, benzyl cyanide, isovaleraldehyde, 3-pentanone, 2-isobutylthaizole, benzaldehyde, isovaleric acid, 1-nitro-3-methylbutane, β-ionone, β-cyclocitral, 6-methyl-5-hepten-2-one, geranial, phenylacetaldehyde, geranylacetone, 2-phenyl ethanol, 1-octen-3-one, and any combination of two or more of these volatile compounds.

25. The method of claim 23, wherein the at least one volatile compound positively associated with liking is selected from the group of volatile compounds consisting of: 1-penten-3-one, trans-2-hexenal, cis-2-penten-1-ol, geranial, 3-methyl-1-butanol, 1-octen-3-one, trans-2-pentenal, isovaleronitrile, trans-3-hexen-1-ol, 1-nitro-3-methylbutane, 6-methyl-5-hepten-2-one, and any combination of two or more of these volatile compounds.

26. The method of claim 24, wherein the at least one volatile compound positively associated with liking is a combination of two or more of the volatile compounds.

27. The method of claim 24, wherein the at least one volatile compound positively associated with liking is a combination of three or more of the volatile compounds.

28. The method of claim 23, wherein the fruit of the heirloom tomato cultivar also comprises a lower amount of at least one volatile compound negatively associated with liking than the amount of that compound in the elite hybrid tomato cultivar.

29. The method of claim 28, wherein the at least one volatile compound negatively associated with liking is selected from the group of volatile compounds consisting of: eugenol, salicylaldehyde, isobutyl acetate, butyl acetate, 2-methylbutanal, and any combination of two or more of these volatile compounds.

30. The method of claim 23, further comprising back-crossing the F1 hybrid tomato plant with one of the parent tomato cultivars.

31. The method of claim 23, wherein the heirloom tomato cultivar is selected from group of cultivars consisting of: Cherry Roma, Matina, Ailsa Craig, Red Calabash, Red Pear, Bloody Butcher, Maglia Rosa Cherry, Brandywine, Tommy Toe, Chadwick Cherry, Livingston's Stone, Super Sioux, St. Pierre, German Queen, Wisconsin 55, Micado Violettor, Livingston's Globe, and Gulf State Market.

32. A hybrid tomato plant produced by backcrossing a hybrid descendent of an ancestor heirloom tomato cultivar and an ancestor elite cultivar with one of the ancestor cultivars, wherein the hybrid tomato plant produces fruit that comprises a higher amount of at least one volatile compound positively associated with liking than the amount of that volatile compound present in fruit produced by the ancestor elite cultivar.

33. The hybrid tomato plant of claim 32, wherein the at least one volatile compound positively associated with liking is selected from the group of volatile compounds consisting of: 1-penten-3-one, trans-2-pentenal, trans-2-heptenal, trans-3-hexen-1-ol, trans-2-hexenal, cis-2-penten-1-ol, 6-methyl-5-hepten-2-ol, nonyl aldehyde, isovaleronitrile, cis-4-decenal, 3-methyl-1-butanol, 2,5-dimethyl-4-hydroxy-3(2H)-furanone, 1-pentanol, methional, benzyl cyanide, isovaleraldehyde, 3-pentanone, 2-isobutylthaizole, benzaldehyde, isovaleric acid, 1-nitro-3-methylbutane, β-ionone, β-cyclocitral, 6-methyl-5-hepten-2-one, geranial, phenylacetaldehyde, geranylacetone, 2-phenyl ethanol, 1-octen-3-one, and any combination of two or more of these volatile compounds.

34. The hybrid tomato plant of claim 32, wherein the at least one volatile compound positively associated with liking is selected from the group of volatile compounds consisting of: 1-penten-3-one, trans-2-hexenal, cis-2-penten-1-ol, geranial, 3-methyl-1-butanol, 1-octen-3-one, trans-2-pentenal, isovaleronitrile, trans-3-hexen-1-ol, 1-nitro-3-methylbutane, 6-methyl-5-hepten-2-one, and any combination of two or more of these volatile compounds.

35. The hybrid tomato plant of claim 33, wherein the fruit of the hybrid tomato plant comprises a higher amount of at least two of the volatile compounds positively associated with liking.

36. The hybrid tomato plant of claim 33, wherein the fruit of the hybrid tomato plant comprises a higher amount of at least three of the volatile compounds positively associated with liking.

37. The hybrid tomato plant of claim 32, wherein the hybrid tomato plant produces fruit that comprises a lower amount of at least one volatile compound negatively associated with liking than the amount of that volatile compound present in the fruit produced by the ancestor elite cultivar.

38. The hybrid tomato plant of claim 37, wherein the at least one volatile compound negatively associated with liking is selected from the group of volatile compounds consisting of: eugenol, salicylaldehyde, isobutyl acetate, butyl acetate, 2-methylbutanal, and any combination of two or more of these volatile compounds.

39. The hybrid tomato plant of claim 32, wherein the fruit of the hybrid tomato plant further comprises a higher sugar content than fruit of the ancestor elite cultivar.

40. The hybrid tomato plant of claim 32, wherein the fruit of the F1 hybrid tomato plant further comprises a sugar to acid ratio from about 8 to about 16.

41. A method of identifying a tomato plant that produces better tasting tomato fruit comprising:
performing a chemical analysis of a tomato fruit from each of a variety of tomato plants, wherein the chemical analysis comprises quantifying an amount of at least one volatile compounds selected from the group of volatile compounds consisting of: 1-penten-3-one, trans-2-hexenal, cis-2-penten-1-ol, geranial, 3-methyl-1-butanol, 1-octen-3-one, trans-2-pentenal, isovaleronitrile, trans-3-hexen-1-ol, 1-nitro-3-methylbutane, 6-methyl-5-hepten-2-one, 2-methylbutanal, butyl acetate, isobutylacetate, and eugenol; and
selecting the tomato plant that produced fruit having the greatest amount of one or more of the compounds positively associated with liking selected from the group of volatile compounds consisting of: 1-penten-3-one, trans-2-hexenal, cis-2-penten-1-ol, geranial, 3-methyl-1-butanol, 1-octen-3-one, trans-2-pentenal, isovaleronitrile, trans-3-hexen-1-ol, 1-nitro-3-methylbutane, and 6-methyl-5-hepten-2-one.

42. The method of claim 41, further comprising selecting a tomato plant that produced fruit having the least amount of one or more of the compounds negatively associated with liking selected from the group of volatile compounds consisting of: 2-methylbutanal, butyl acetate, isobutylacetate, and eugenol.

43. The method of claim 41, comprising quantifying an amount of two or more of the volatile compounds and selecting the tomato plant that produced fruit having the greatest amount of two or more of the compounds.

44. A fruit of the hybrid tomato plant of claim 5.

45. A fruit of the F1 hybrid tomato plant of claim 13.

46. A fruit of the hybrid tomato plant of claim 22.

47. A hybrid tomato plant produced by the method of claim 23.

48. A fruit of the hybrid tomato plant of claim 32.
